(19) United States
(12) Patent Application Publication
(10) Pub. No.: US 2024/0415888 A1
(43) Pub. Date: Dec. 19, 2024

(54) ANTI-TSHR MULTI-SPECIFIC ANTIBODIES AND USES THEREOF

(71) Applicants: Memorial Sloan Kettering Cancer Center, New York, NY (US); Memorial Hospital for Cancer and Allied Diseases, New York, NY (US); Sloan-Kettering Institute for Cancer Research, New York, NY (US)

(72) Inventors: Nai-Kong V. Cheung, New York, NY (US); James A. Fagin, New York, NY (US); Hong Fen Guo, New York, NY (US); Yuchen Jin, New York, NY (US); Jeffrey Knauf, New York, NY (US); Gnana Krishnamoorthy, New York, NY (US); Yiwei Liu, New York, NY (US); Brian Santich, New York, NY (US)

(21) Appl. No.: 18/703,318

(22) PCT Filed: Oct. 17, 2022
(86) PCT No.: PCT/US2022/046842

§ 371 Date: Apr. 19, 2024

Related U.S. Application Data
(60) Provisional application No. 63/257,694, filed on Oct. 20, 2021.

(51) Int. Cl. A61K 35/17; A61K 39/00; A61P 35/00; C07K 16/28; C07K 16/32

(52) U.S. Cl. CPC A61K 35/17; A61K 39/4611; A61K 39/464406; A61K 39/464411; A61P 35/00; C07K 16/2809; C07K 16/2869; C07K 16/32; A61K 2039/505; C07K 2317/31; C07K 2317/51; C07K 2317/515; C07K 2317/522; C07K 2317/622

(57) ABSTRACT

The present technology provides anti-Thyroid Stimulating Hormone Receptor (TSHR) multi-specific (e.g., bispecific) immunoglobulin-related compositions and methods of using the same to treat TSHR-associated pathologies including, but not limited to, thyroid cancers, T-ALL (T lineage acute lymphoblastic leukemia), multiple myeloma and Grave's disease. Kits for use in practicing the methods are also provided.

### CROSS-REFERENCE TO RELATED APPLICATIONS

This application claims the benefit of and priority to U.S. Provisional Application No. 63/257,694, filed Oct. 20, 2021, which is incorporated herein by reference in its entirety.

### STATEMENT OF GOVERNMENT SUPPORT

This invention was made with government support under grant number CA249663 awarded by the National Institutes of Health (NIH). The government has certain rights in the invention.

### TECHNICAL FIELD

The present technology relates to anti-Thyroid Stimulating Hormone Receptor (TSHR) multi-specific (e.g., bispecific) immunoglobulin-related compositions and methods of using the same to treat TSHR-associated pathologies including, but not limited to, thyroid cancers, T-ALL (T lineage acute lymphoblastic leukemia), multiple myeloma and Grave's disease. Kits for use in practicing the methods are also provided.

### BACKGROUND

The following description of the background of the present technology is provided simply as an aid in understanding the present technology and is not admitted to describe or constitute prior art to the present technology.

TSHR is the thyroid-stimulating hormone (TSH) or thyrotropin receptor for heterodimeric glycoprotein hormone (GPHA2:GPHB5) or thyrostimulin. The activity of this receptor is mediated by G proteins which activate adenylate cyclase (Class A GPCR). Among growth factor receptors, TSHR (LGR3, on chromosome 14q31.1) expression is one of the most restricted, found primarily in the thyroid gland, but also in the retro-orbital and adipose tissues with direct implications for Graves' disease and adipogenesis. Additionally, mRNA transcripts could be detected in various regions of the normal brain, select cells in the hematopoietic system (T regs and plasmablasts), thymus, adrenal, testes, ovaries, and placenta. TSHR is also expressed in osteoclast and osteoblast precursors and mediates effects of TSH on bone remodeling. Importantly, it is not secreted into the blood. TSHR is highly expressed in T cell acute lymphoblastic leukemia (T-ALL), multiple myeloma and thyroid cancer, although low levels have been reported for lung, CRC, gastric, liver, pancreatic, urothelial, breast and ovarian cancers (FIG. 8). It is also one of the most mutated GPCR in human cancers and activating TSHR mutations are primarily seen in autonomously functioning thyroid adenomas, in a subset of differentiated and, less frequently, poorly differentiated thyroid cancers.

For thyroid cancer, chimeric antigen receptor (CAR) gene modified T cells have been previously described, but none of these biologics have been translated to the clinic. Treatment for thyroid cancer recurrent after, or resistant to, tyrosine kinase inhibitors (TKIs) remain unsatisfactory. Similarly effective salvage therapy for T-ALL and myeloma that fail frontline therapy remains major unmet need. Thus, there is an urgent need for therapeutic methods that effectively target TSHR (+) cancers, such as thyroid cancers, T-ALL, and multiple myeloma.

### SUMMARY OF THE PRESENT TECHNOLOGY

In one aspect, the present disclosure provides a multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain are covalently bonded to one another, and wherein: (a) each of the first polypeptide chain and the fourth polypeptide chain comprises in the N-terminal to C-terminal direction: (i) a light chain variable domain of a first immunoglobulin that is capable of specifically binding to a first epitope; (ii) a light chain constant domain of the first immunoglobulin; (iii) a flexible peptide linker comprising the amino acid sequence (GGGGS)₃; and (iv) a light chain variable domain of a second immunoglobulin that is linked to a complementary heavy chain variable domain of the second immunoglobulin, or a heavy chain variable domain of a second immunoglobulin that is linked to a complementary light chain variable domain of the second immunoglobulin, wherein the light chain and heavy chain variable domains of the second immunoglobulin are capable of specifically binding to a second epitope, and are linked together via a flexible peptide linker comprising the amino acid sequence (GGGGS)₆ to form a single-chain variable fragment; and (b) each of the second polypeptide chain and the third polypeptide chain comprises in the N-terminal to C-terminal direction: (i) a heavy chain variable domain of the first immunoglobulin that is capable of specifically binding to the first epitope; and (ii) a heavy chain constant domain of the first immunoglobulin; and wherein the heavy chain variable domain of the first immunoglobulin is SEQ ID NO: 57 or SEQ ID NO: 59, wherein the light chain variable domain of the first immunoglobulin is SEQ ID NO: 58 or SEQ ID NO: 60, wherein the heavy chain variable domain of the second immunoglobulin is SEQ ID NO: 61 or SEQ ID NO: 62 and wherein the light chain variable domain of the second immunoglobulin is SEQ ID NO: 63 or SEQ ID NO: 64. In some embodiments of the multi-specific antibody, each of the first polypeptide chain and the fourth polypeptide chain comprises the amino acid sequence of SEQ ID NO: 1, and each of the second polypeptide chain and the third polypeptide chain comprises the amino acid sequence of SEQ ID NO: 3. In other embodiments of the multi-specific antibody, each of the first polypeptide chain and the fourth polypeptide chain comprises the amino acid sequence of SEQ ID NO: 27, and each of the second polypeptide chain and the third polypeptide chain comprises the amino acid sequence of SEQ ID NO: 29.

In one aspect, the present disclosure provides a heterodimeric multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃; and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ to form a single-chain variable fragment; (b) the second polypeptide chain comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide chain comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide chain comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃; and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ to form a single-chain variable fragment; wherein VL-1 or VL-3 comprises a VL amino acid sequence selected from any one of SEQ ID NO: 58 or SEQ ID NO: 60, wherein VH-1 or VH-3 comprises a VH amino acid sequence selected from any one of SEQ ID NO: 57 or SEQ ID NO: 59, wherein VH-2 or VH-4 comprises a VH amino acid sequence selected from any one of SEQ ID NO: 61 or SEQ ID NO: 62, and wherein VL-2 or VL-4 comprises a VL amino acid sequence selected from any one of SEQ ID NO: 63 or SEQ ID NO: 64. In some embodiments of the multi-specific antibody, the first polypeptide chain or the fourth polypeptide chain comprises the amino acid sequence of SEQ ID NO: 1, and the second polypeptide chain or the third polypeptide chain comprises the amino acid sequence of SEQ ID NO: 3. In other embodiments of the multi-specific antibody, the first polypeptide chain or the fourth polypeptide chain comprises the amino acid sequence of SEQ ID NO: 27, and the second polypeptide chain or the third polypeptide chain comprises the amino acid sequence of SEQ ID NO: 29.

In any and all embodiments of the anti-TSHR multi-specific antibody disclosed herein, the antibody or antigen binding fragment binds to a TSHR polypeptide comprising amino acids 22-260 of SEQ ID NO: 74.

In one aspect, the present disclosure provides a method for treating a TSHR-positive cancer in a subject in need thereof comprising administering to the subject an effective amount of any and all embodiments of the anti-TSHR multi-specific antibody disclosed herein. In certain embodiments, the TSHR-positive cancer is thyroid cancer, T lineage acute lymphoblastic leukemia, multiple myeloma, lung cancer, colorectal cancer, gastric cancer, liver cancer, pancreatic cancer, urothelial cancer, breast cancer, or ovarian cancer. Additionally or alternatively, in some embodiments, the TSHR-positive cancer is resistant to a RET inhibitor, a NTRK inhibitor, an ALK inhibitor, a RAF inhibitor, or a MEK kinase inhibitor.

In another aspect, the present disclosure provides a method for treating a TSHR-associated pathology in a subject in need thereof comprising administering to the subject an effective amount of any and all embodiments of the anti-TSHR multi-specific antibody disclosed herein, wherein the TSHR-associated pathology is Graves' disease, or thyroid-associated ophthalmopathy (TAO).

In yet another aspect, the present disclosure provides a method for modulating weight gain in a subject in need thereof comprising administering to the subject an effective amount of any and all embodiments of the anti-TSHR multi-specific antibody described herein.

In another aspect, the present disclosure provides a method for decreasing bone remodeling to treat osteoporosis in a subject in need thereof comprising administering to the subject an effective amount of any and all embodiments of the anti-TSHR multi-specific antibody described herein.

Additionally or alternatively, in some embodiments, the methods of the present technology further comprise administering to the subject an effective amount of a multi-specific antibody that specifically binds to HER2 and T cells. In some embodiments, the multi-specific antibody that specifically binds to HER2 and T cells comprises a light chain amino acid sequence of SEQ ID NO: 54 and a heavy chain amino acid sequence of SEQ ID NO: 56.

In one aspect, the present disclosure provides an ex vivo armed T cell that is coated or complexed with an effective amount of any and all embodiments of the anti-TSHR multi-specific antibody disclosed herein. Additionally or alternatively, in some embodiments, the ex vivo armed T cell is further coated or complexed with an effective amount of a multi-specific antibody that specifically binds to HER2 and T cells. In certain embodiments, the multi-specific antibody that specifically binds to HER2 and T cells comprises a light chain amino acid sequence of SEQ ID NO: 54 and a heavy chain amino acid sequence of SEQ ID NO: 56.

Also disclosed herein are methods for treating a TSHR-associated cancer in a subject in need thereof comprising administering to the subject an effective amount of any and all embodiments of the ex vivo armed T cells disclosed herein. In some embodiments, the ex vivo armed T cell is a αβ-T cell or a γδ-T cell. Additionally or alternatively, in some embodiments, the ex vivo armed T cell is obtained from a third party donor (e.g., allogeneic), or is obtained from the subject in need thereof (e.g., autologous). The ex vivo armed T cell may be cryopreserved or freshly harvested from a donor. Additionally or alternatively, in some embodiments, the TSHR-positive cancer is resistant to a RET inhibitor, a NTRK inhibitor, an ALK inhibitor, a RAF inhibitor, or a MEK kinase inhibitor.

Additionally or alternatively, in some embodiments, the methods of the present technology comprise administering to the subject an effective amount of a first population of ex vivo armed T cells that is coated or complexed with an effective amount of any and all embodiments of the anti-TSHR×CD3 multi-specific antibody disclosed herein and a second population of ex vivo armed T cells that is coated or complexed with an effective amount of a multi-specific antibody that specifically binds to HER2 and T cells, wherein the second population of ex vivo armed T cells is distinct from the first population of ex vivo armed T cells. In certain embodiments, the multi-specific antibody present on the second population of ex vivo armed T cells comprises a light chain amino acid sequence of SEQ ID NO: 54 and a heavy chain amino acid sequence of SEQ ID NO: 56.

### BRIEF DESCRIPTION OF THE DRAWINGS

FIGS. 1A-1B show adaptive responses of RTK, BRAF or RAS-driven thyroid cancers to MAPK inhibitors. FIG. 1A: RAF or MEK inhibitors de-repress HER3 and HER2 transcription. Autocrine-secreted NRG-1 binds to HER3, triggers HER3/HER2 heterodimerization and receptor phosphorylation, reactivating MAPK and PI3K signaling. FIG. 1B: MAPK pathway activation dampens expression of TSHR by inhibiting expression of the lineage transcription factors Foxe1 and Pax8, and by inhibiting signaling nodes in the adenylyl cyclase-cAMP-PKA pathway (not shown). These effects are reversed by RAF or MEK inhibitors.

FIG. 2A: Expression of TSHR in thyroid cancers with the indicated histologies (from NCBI GEO datasets GSE29265, GSE336301, 2, GSE651443, GSE760394). FIG. 2B: TSHR expression in normal thyroid compared to TCGA papillary carcinoma (PTC) dataset sorted by the indicated drivers (BRAF^{V600E}, N/K/H-RAS mutants and non-BRAF & RAS driven PTC). FIG. 2C: TSHR expression in normal tissues from Proteomics DB and MOPED. FIG. 2D: Expression of TSHR in various genetically engineered mouse models (GEMMs) of thyroid cancer with the indicated genotypes. FIG. 2E: Effects of the MEK inhibitor CH5126766 (CKI) on TSHR mRNA in GEMMs of thyroid cancer. FIG. 2F: RNAseq of patient biopsies collected from RAS or BRAFV600E mutant thyroid cancers prior to and while on treatment with BRAF inhibitors (dabrafenib or vemurafenib) or the combination of BRAF and HER3 kinase inhibitors (dabrafenib+lapatinib or vemurafenib+CDX-3379). FIG. 2G: Panel of RAS mutant thyroid cancer cells showing increased HER2 (5/10), and TSHR (10/10) expression as assessed by median fluorescence intensity by flow after treatment with trametinib for 48 and 72 h. FIG. 2H: ML1 cells showing increased HER2 and TSHR expression by RT-PCR and flow upon treatment with trametinib.

FIG. 3: Diagram illustrating HER2/CD3 and TSHR/CD3 bispecific antibodies (BsAbs) in the IgG-[L]-scFv (2+2) format.

FIGS. 4A-4D show TSHR-BsAb specific binding and functional activity. FIG. 4A: HEK-293T cells transfected with various TSHR expression vectors demonstrating M22/CD3 and K1-70/CD3 BsAb binding by flow. FIGS. 4B-4D: CAMP levels in 8505C cells transduced with TSHR expression vector as measured by ELISA. M22/CD3 BsAb induces cAMP in a dose-dependent manner (in ng/ml) comparable to bovine TSH (in mIU/ml). The K1-70 Ab blocks TSH (10 mIU/ml)-induced CAMP in a dose-dependent manner.

FIGS. 5A-5C show TSHR-BsAb cell killing experiments in vitro. FIG. 5A: M22 and K1-70-BsAb flow showing binding of TSHR-BsAb to THJ560 and BHT101 thyroid cancer cells ectopically expressing TSHR. FIG. 5B: TSHR-BsAbs mediate T-cell killing in TSHR overexpressing thyroid cancer cells compared to control BsAb as determined by ⁵¹Cr release assay. FIG. 5C: ⁵¹Cr release assay in ML1 cells−/+trametinib, showing increased TSHR-BsAb mediated T-cell killing upon treatment with trametinib compared to DMSO.

FIGS. 6A-6C show TSHR-BsAb cell killing in vivo. FIG. 6A: Schema for in vivo experiments showing timing of low iodine diet, mouse thyroid ablation with iodine-131, ML1 cell implantation and schedule of treatments with trametinib and BsAb-armed T-cells in BRG mice. FIG. 6B: Tumor growth curves of SC-implanted ML1 tumors in BRG mice after treatment of trametinib, TSHR BsAbs (M22 or K1-70) and the combination. Dotted line represents the mean tumor size in each group (n=3/group). FIG. 6C: CD3 staining by IHC of ML1 xenografts following the indicated treatments. Combination treatment with trametinib and TSHR BsAbs (M22 or K1-70) showing high CD3+ T cell infiltration. Left: 2× magnification; Right: 20× magnification.

FIGS. 7A-7C show HER2 BsAb cell killing experiments in vitro & in vivo. FIG. 7A: Her2-BsAb mediated T-cell killing assayed by ⁵¹Cr release assay in a panel of thyroid cancer cells with the indicated genotypes. FIG. 7B: Treatment effect of ML1 implanted xenograft tumors in BRG mice showing tumor shrinkage after treatment with trametinib and HER2 BsAbs. Dotted line represents the mean tumor size in each group (n=3/group). FIG. 7C: CD3 staining by IHC of the ML1 xenografts treated with trametinib and/or HER2-BsAb (Top), or with the indicated controls (Bottom). Left: 2× magnification; Right: 20× magnification.

FIG. 8 shows TSHR gene expression in human cancer cell lines.

FIG. 9 shows bioluminescence images 1 and 6 days post injection of Luc (+) T cells armed with the indicated bispecific antibodies in mice treated with vehicle (Top) or trametinib (Bottom) showing higher T cell trafficking into tumors (circled in red color) from mice receiving TSHR or HER2 BsAb in the presence of trametinib.

FIGS. 10A-10H show nucleic acid and amino acid sequences of the M22 anti-TSHR multi-specific antibody compositions of the present technology (represented as SEQ ID NOs: 1-26). V_{L} domains are indicated in boldface; V_{H} domains are indicated in italics and underlined; Linkers are italicized.

FIGS. 11A-11H show nucleic acid and amino acid sequences of the K1-70 anti-TSHR multi-specific antibodies of the present technology (represented as SEQ ID NOs: 27-52). V_{L} domains are indicated in boldface; V_{H} domains are indicated in italics and underlined; Linkers are italicized.

FIG. 12A shows the nucleic acid and amino acid sequences of the light chain of anti-HER2×CD3 bispecific antibody (Biclone120DS) represented as SEQ ID NOs: 53-54, respectively. FIG. 12B shows the nucleic acid and amino acid sequences of the heavy chain of anti-HER2×CD3 bispecific antibody (Biclone120DS) represented as SEQ ID NOs: 55-56, respectively.

### DETAILED DESCRIPTION

It is to be appreciated that certain aspects, modes, embodiments, variations and features of the present technology are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Recurrent/metastatic thyroid cancers refractory to radioiodine therapy are primarily driven by oncoproteins that signal along the MAPK pathway. Non-overlapping point mutations of BRAF or RAS, or fusions of receptor tyrosine kinase (RTK) are present in ˜90% of differentiated thyroid cancers (DTC) and ˜70% to 80% of poorly differentiated and anaplastic thyroid cancers (ATC). In response to treatment with RAF or MEK inhibitors thyroid cancers undergo two major types of adaptive changes that modulate the response to therapy primarily by increasing abundance of the plasma membrane receptors HER3, HER2 and TSHR.

Thyroid cancer are generally “cold” tumors with few tumor infiltrating lymphocytes, hence poor response to immune checkpoint inhibitors. Furthermore the thyroid tumor microenvironment is filled with myeloid suppressor cells that derail most conventional T cell based approaches. Since many thyroid tumors are “cold”, devoid of de novo tumor infiltrating lymphocytes (TILs), T cells need to be driven, e.g. by chimeric antigen receptor (CAR). Most recently, CAR T cells specific for TSHR was successfully built using the V_{H} and V_{L} sequences from M22 and K1-70 (Li H et al., medRxiv: 2021.05.15.21256466). While M22-CAR T therapy was not successful, K1-70 CAR T therapy was marginally effective in prolonging survival in preclinical models, partly because of tonic activation and T cell exhaustion by the CAR. Li H et al., medRxiv: 2021.05.15.21256466.

The present disclosure provides T-cell engaging anti-TSHR multi-specific antibodies based on the IgG [L]-scFv antibody format that include M22 and K1-70 sequences, which show robust in vitro and in vivo antitumor properties. The anti-TSHR multi-specific immunoglobulin-related compositions of the present technology were highly effective in driving human T cells into thyroid tumors, and ablate thyroid tumors even when they were large and established. The present disclosure also demonstrates that thyroid cancers treated with MAP kinase inhibitors upregulated TSHR and HER2 as part of their differentiation and escape pathways, thus rendering these tumors even more susceptible to TSHR and HER2 mediated T-cell engaging BsAb ablation.

### Definitions

The definitions of certain terms as used in this specification are provided below. Unless defined otherwise, all technical and scientific terms used herein generally have the same meaning as commonly understood by one of ordinary skill in the art to which this present technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes native antibodies, monoclonal antibodies, human antibodies, humanized antibodies, camelised antibodies, multi-specific antibodies, bispecific antibodies, chimeric antibodies, Fab, Fab′, single chain V region fragments (scFv), single domain antibodies (e.g, nanobodies and single domain camelid antibodies), VNAR fragments, Bi-specific T-cell engager antibodies, minibodies, disulfide-linked Fvs (sdFv), and anti-idiotypic (anti-id) antibodies, intrabodies, fusion polypeptides, unconventional antibodies and antigen-binding fragments of any of the above. See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target protein (e.g., TSHR) will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, human antibodies, humanized antibodies, chimeric antibodies, recombinant antibodies, multi-specific antibodies, bispecific antibodies, etc.,) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region (A“F(ab′)₂” fragment can be split into two individual Fab′ fragments); (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341:544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies (dscFvs); linear antibodies; single-chain antibody molecules; and multi-specific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody comprises two antigen binding moieties, wherein one of the two antigen binding moieties includes an antibody fragment (e.g., Fab, F(ab′), F(ab′)₂, Fd, Fv, dAB, scFv, etc.) having a V_{H} region and/or a V_{L} region that contain CDRs from a first monoclonal antibody, and the other antigen binding moiety includes an antibody fragment (e.g., Fab, F(ab′), F(ab′)₂, Fd, Fv, dAB, scFv, etc.) having a V_{H} region and a V_{L} region that contain CDRs from a second monoclonal antibody. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “antibody-dependent cell-mediated cytotoxicity” or “ADCC”, refers to a mechanism of cell-mediated immune defense whereby an effector cell of the immune system actively lyses a target cell, such as a tumor cell, whose membrane-surface antigens have been bound by antibodies such as the anti-TSHR multi-specific antibodies of the present technology.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a TSHR polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto. Any of the above-noted antibody fragments are obtained using conventional techniques known to those of skill in the art, and the fragments are screened for binding specificity and neutralization activity in the same manner as are intact antibodies.

As used herein, “binding affinity” means the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “CDR grafting” means replacing at least one CDR of an “acceptor” antibody by a CDR “graft” from a “donor” antibody possessing a desirable antigen specificity. As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240:1041-1043, 1988; Liu et al., Proc Natl Acad Sci USA 84:3439-3443, 1987; Liu et al., J. Immunol 139:3521-3526, 1987; Sun et al., Proc Natl Acad Sci USA 84:214-218, 1987; Nishimura et al., Cancer Res 47:999-1005, 1987; Wood et al., Nature 314:446-449, 1885; and Shaw et al., J. Natl. Cancer Inst. 80:1553-1559, 1988.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, the term “consensus FR” means a framework (FR) antibody region in a consensus immunoglobulin sequence. The FR regions of an antibody do not contact the antigen.

As used herein, the term “constant region” or “constant domain” is interchangeable and has its meaning common in the art. The constant region is an antibody portion, e.g, a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc Natl Acad Sci USA, 90:6444-6448 (1993).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, the epitope is a conformational epitope or a non-conformational epitope. To screen for anti-TSHR antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-TSHR antibody binds the same site or epitope as an anti-TSHR antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of TSHR protein can be used in competition assays with the test antibodies or with a test antibody and an antibody with a characterized or known epitope. An epitope can be, e.g., contiguous amino acids of a polypeptide (linear or contiguous epitope) or an epitope can, e.g., come together from two or more noncontiguous regions of a polypeptide or polypeptides (conformational, non-linear, discontinuous, or non-contiguous epitope).

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g, by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, the term “gene” means a segment of DNA that contains all the information for the regulated biosynthesis of an RNA product, including promoters, exons, introns, and other untranslated regions that control expression.

As used herein, “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588 (2): 288-297 (2014). By way of example, a humanized version of a murine antibody to a given antigen has on both of its heavy and light chains (1) constant regions of a human antibody; (2) framework regions from the variable domains of a human antibody; and (3) CDRs from the murine antibody. When necessary, one or more residues in the human framework regions can be changed to residues at the corresponding positions in the murine antibody so as to preserve the binding affinity of the humanized antibody to the antigen. This change is sometimes called “back mutation.” Similarly, forward mutations may be made to revert back to murine sequence for a desired reason, e.g., stability or affinity to antigen.

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the terms “immunospecifically binds,” “immunospecifically recognizes,” “specifically binds,” and “specifically recognizes” are analogous terms in the context of antibodies and refer to antibodies and antigen-binding fragments thereof that bind to an antigen (e.g., epitope or immune complex) via the antigen-binding sites as understood by one skilled in the art, and does not exclude cross-reactivity of the antibody or antigen binding fragment with other antigens.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “ligand” refers to a molecule that binds to a receptor. In particular, the ligand binds a receptor on another cell, allowing for cell-to-cell recognition and/or interaction.

As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence (GGGGS)ₙ, wherein n is at least 1, 2, 3, 4, 5, 6, 7, 8, 9, 10, 11, 12, 12, 14, or 15.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “nucleic acid” or “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, PA).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)). Bird et al. (1988) Science 242:423-426 and Huston et al. (1988) Proc Natl Acad Sci 85:5879-5883. Such single-chain antibodies can be prepared by recombinant techniques or enzymatic or chemical cleavage of intact antibodies.

The V_{H} and V_{L} domains are either joined directly or joined by a peptide-encoding linker (e.g, about 10, 15, 20, 25 amino acids), which connects the N-terminus of the V_{H} with the C-terminus of the V_{L}, or the C-terminus of the V_{H} with the N-terminus of the V_{L}. In some embodiments, the linker is usually rich in glycine for flexibility, as well as serine or threonine for solubility. The linker can link the heavy chain variable region and the light chain variable region of the extracellular antigen binding domain.

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al, Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al, Hybridoma (Larchmt) 27 (6): 455-51 (2008); Peter et al, J Cachexia Sarcopenia Muscle (2012); Shieh et al, J Imunol 183 (4): 2277-85 (2009); Giomarelli et al, Thromb Haemost 97 (6): 955-63 (2007); Fife et al, J Clin Invst 116 (8): 2252-61 (2006); Brocks et al, Immunotechnology 3 (3): 173-84 (1997); Moosmayer et al, Ther Immunol 2 (10): 31-40 (1995). Agonistic scFvs having stimulatory activity have been described (see, e.g., Peter et al, J Biol Chem 25278 (38): 36740-7 (2003); Xie et al, Nat Biotech 15 (8): 768-71 (1997); Ledbetter et al, Crit Rev Immunol 17 (5-6): 427-55 (1997); Ho et al, Bio Chim Biophys Acta 1638 (3): 257-66 (2003)).

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a TSHR polypeptide), or an epitope on a particular polypeptide, without substantially binding to any other polypeptide, or polypeptide epitope.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

It is also to be appreciated that the various modes of treatment of medical diseases and conditions as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-TSHR antibodies described herein are contemplated. Such modifications can be introduced to improve the binding affinity and/or other biological properties of the antibody, for example, to render the encoded amino acid aglycosylated, or to destroy the antibody's ability to bind to Clq, Fc receptor, or to activate the complement system. Amino acid sequence variants of an anti-TSHR antibody are prepared by introducing appropriate nucleotide changes into the antibody nucleic acid, by peptide synthesis, or by chemical modifications. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. In some embodiments, the Fc regions of the antibodies have two amino acid substitutions, Leu234Ala and Leu235Ala (so called LALA mutations) to eliminate FcγRIIa binding. The LALA mutations are commonly used to alleviate the cytokine induction from T cells, thus reducing toxicity of the antibodies (Wines B D, et al., J Immunol 164.5313-5318 (2000)). The modification also includes the change of the pattern of glycosylation of the protein. The sites of greatest interest for substitutional mutagenesis include the hypervariable regions, but FR alterations are also contemplated.

Conservative amino acid substitutions are amino acid substitutions that change a given amino acid to a different amino acid with similar biochemical properties (e.g., charge, hydrophobicity and size). Generally, genetically encoded amino acids are divided into families: (1) acidic, comprising aspartate and glutamate; (2) basic, comprising arginine, lysine, and histidine; (3) non-polar, comprising isoleucine, alanine, valine, proline, methionine, leucine, phenylalanine, tryptophan; and (4) uncharged polar, comprising cysteine, threonine, glutamine, glycine, asparagine, serine, and tyrosine. In addition, an aliphatic-hydroxy family comprises serine and threonine. In addition, an amide-containing family comprises asparagine and glutamine. In addition, an aliphatic family comprises alanine, valine, leucine and isoleucine. In addition, an aromatic family comprises phenylalanine, tryptophan, and tyrosine. Finally, a sulfur-containing side chain family comprises cysteine and methionine. As an example, one skilled in the art would reasonably expect an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. “Conservative substitutions” are shown in the Table below.

Amino Acid Substitutions

| Original | Exemplary | Conservative |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

### Thyroid Cancer

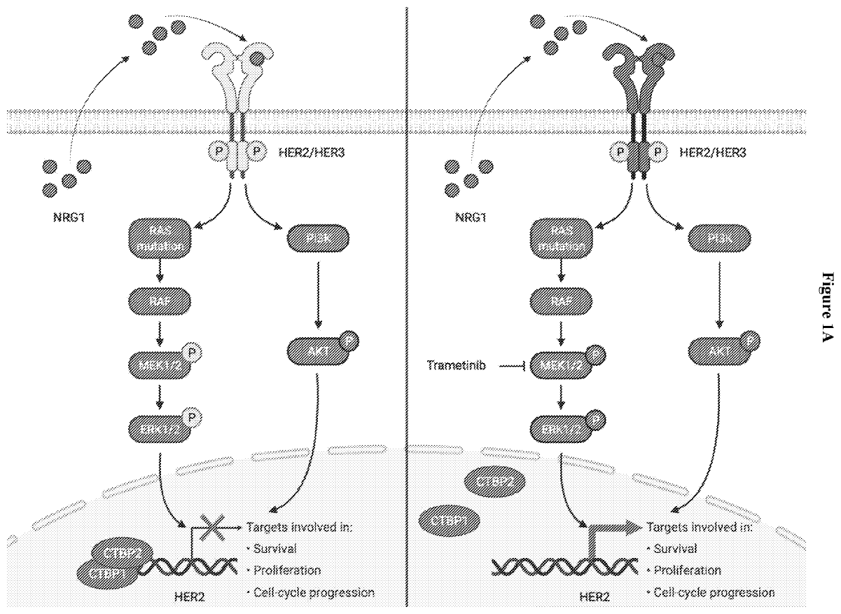

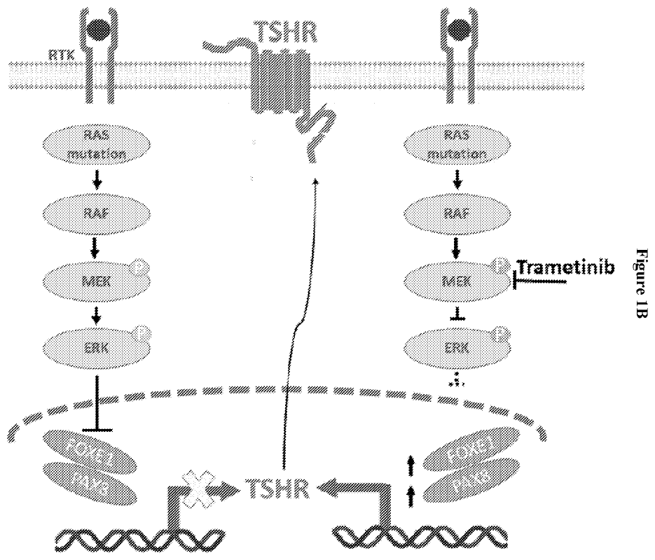

Treatment of BRAF^{V600E}-radioiodine (RAI) refractory metastatic differentiated thyroid cancers with the RAF kinase inhibitor vemurafenib induced partial response (PR) in 38% of patients who had not previously received VEGF inhibitors, and 28% in those who did (Brose M S et al., Lancet Oncol 17:1272-82 (2016)). These responses are attenuated compared to BRAF-mutant melanomas (Flaherty et al., N. Engl. J. Med 363:809-819 (2010); Chapman P B et al., N. Engl. J. Med 364:2507-2516, 2011), largely due to the induction of HER2/HER3 gene expression and re-activation of MAPK signaling through relief of negative feedback in preclinical thyroid cancer models (Montero-Conde C et al., Cancer Discov 3:520-533 (2013); FIG. 1). Consistent with this thyroid cancer patients treated with the combination of BRAF inhibitor (dabrafenib)+dual inhibitor of HER2/neu and epidermal growth factor receptor (EGFR) (lapatinib) had overall response rates of ˜60%, which were quite durable (Sherman E J et al., J Clin Oncol 35:6085 (2017)), although 55% of them eventually progressed. Adaptive changes resulting in increased membrane levels of HER2/HER3 after inhibition of the MAPK pathway also occurs in mutant RAS driven thyroid cancers.

### TSHR

TSHR is a therapeutic target in Graves' disease and its extrathyroidal manifestations such as thyroid-associated ophthalmopathy (TAO). The underlying pathophysiology is the loss of immune tolerance to TSHR leading to the generation of activating antibodies specific for the receptor. Binding affinity (Kd) of TSH for TSHR is 5×10⁻⁸ M (50 nM) at pH 7.5. TSHR is a member of a family of cell surface GPCRs that include luteinizing hormone (LH) and follicle stimulating hormone (FSH) receptors. It comprises a multimeric structure with the ligand-binding site located in the amino-terminus. One gene encodes the receptor which is translated into a single peptide undergoing cleavage into constituent subunits connected by disulfide bonds. The extracellular TSHR domain is cleaved by a cell surface metalloproteinase, the identity of which remains uncertain. This cleaved fragment is particularly immunogenic and is likely responsible for the generation of thyroid stimulating immunoglobulins (TSI). Once TSH binds to TSHR, the receptor is activated and endocytosed, leading to downstream signaling before degradation or recycling. Current treatment for Graves' disease include antithyroid drugs, radioiodine ablation and surgical thyroidectomy. Treatment for TAO is still investigational including IGF-IR or IL-6 blockade and anti-B lymphocyte approaches.

### Anti-TSHR Immunoglobulin-Related Compositions of the Present Technology

The present technology describes methods and compositions for the generation and use of anti-TSHR immunoglobulin-related compositions (e.g., anti-TSHR multi-specific antibodies or antigen binding fragments thereof). The anti-TSHR immunoglobulin-related compositions of the present disclosure may be useful in the diagnosis, or treatment of TSHR-associated pathologies. Anti-TSHR multi-specific immunoglobulin-related compositions within the scope of the present technology include, e.g., but are not limited to, monoclonal, chimeric, humanized, bispecific antibodies and diabodies that specifically bind the target TSHR polypeptide, a homolog, derivative or a fragment thereof. The present disclosure also provides antigen binding fragments of any of the anti-TSHR multi-specific antibodies disclosed herein, wherein the antigen binding fragment is selected from the group consisting of Fab, F(ab)′2, Fab′, scFv, and Fv.

The V_{H} and V_{L} amino acid sequences of the M22 anti-TSHR antibody are:

(SEQ ID NO: 57)
QVQLVQSGAEVKKPGESLKISCRGSGYRFTSYWINWVRQLPGKGL
EWMGRIDPTDSYTNYSPSFKGHVTVSADKSINTAYLQWSSLKAS
DTGMYYCARLEPGYSSTWSVNWGQGTLVTVSS,
and
(SEQ ID NO: 58)
LTVLTQPPSVSGAPRQRVTISCSGNSSNIGNNAVNWYQQLPGKA
PKLLIYYDDQLPSGVSDRFSGSRSGTSASLAIRGLQSEDEADYY
CTSWDDSLDSQLFGGGTRLTVL,

respectively.

The V_{H} and V_{L} amino acid sequences of the K1-70 anti-TSHR antibody are:

(SEQ ID NO: 59)
EVQLVQSGAEVKKPGQSLKISCKASGYSLTDNWIGWVRQKPGKGL
EWMGIIYPGDSDTRYSPSFQGQVTISADKSINTAYLOWSSLKAS
DTAIYYCVGLDWNYNPLRYWGPGTLVTVSS,
and
(SEQ ID NO: 60)
QSVLTQPPSVSAAPGQKVTISCSGSSSDIGSNYVSWYQQFPGTAP
KLLIYDNNKRPSAIPDRFSGSKSGTSATLGITGLQTGDEADYYC
GTWDSRLGIAVFGGGTQLTVL,

respectively.

Exemplar V_{H} amino acid sequences of the huOKT3 anti-CD3 antibodies include:

(SEQ ID NO: 61)
QVQLVQSGGGVVQPGRSLRLSCKASGYTFTRYTMHWVRQAPGKCL
EWIGYINPSRGYTNYNQKFKDRFTISRDNSKNTAFLQMDSLRPED
TGVYFCARYYDDHYSLDYWGQGTPVTVSS,
and
(SEQ ID NO: 62)
QVQLVQSGGGVVQPGRSLRLSCKASGYTFTRYTMHWVRQAPGKGL
EWIGYINPSRGYTNYNQKFKDRFTISRDNSKNTAFLQMDSLRPED
TGVYFCARYYDDHYSLDYWGQGTPVTVSS.

Exemplar V_{L} amino acid sequences of the huOKT3 anti-CD3 antibodies include:

(SEQ ID NO: 63)
DIQMTQSPSSLSASVGDRVTITCSASSSVSYMNWYQQTPGKAPKR
WIYDTSKLASGVPSRFSGSGSGTDYTFTISSLQPEDIATYYCQQW
SSNPFTFGCGTKLQITR,
and
(SEQ ID NO: 64)
DIQMTQSPSSLSASVGDRVTITCSASSSVSYMNWYQQTPGKAPKR
WIYDTSKLASGVPSRFSGSGSGTDYTFTISSLQPEDIATYYCQQW
SSNPFTFGQGTKLQITR.

In one aspect, the present disclosure provides a multi-specific antibody or antigen binding fragment comprising a TSHR antigen binding domain and a CD3 antigen binding domain, wherein (a) the TSHR antigen binding domain includes a heavy chain immunoglobulin variable domain (V_{H}) amino acid sequence selected from the group consisting of: SEQ ID NO: 57 and SEQ ID NO: 59, and a light chain immunoglobulin variable domain (V_{L}) amino acid sequence selected from the group consisting of: SEQ ID NO: 58 and SEQ ID NO: 60, and (b) the CD3 antigen binding domain includes a heavy chain immunoglobulin variable domain (V_{H}) amino acid sequence selected from the group consisting of: SEQ ID NO: 61 and SEQ ID NO: 62 and a light chain immunoglobulin variable domain (V_{L}) amino acid sequence selected from the group consisting of: SEQ ID NO: 63 and SEQ ID NO: 64.

In any of the above embodiments, the multi-specific antibody further comprises a Fc domain of any isotype, e.g., but are not limited to, IgG (including IgG1, IgG2, IgG3, and IgG4), IgA (including IgA1 and IgA2), IgD, IgE, or IgM, and IgY. Non-limiting examples of constant region sequences include:

Human IgD constant region,
Uniprot: P01880
(SEQ ID NO: 65)
APTKAPDVFPIISGCRHPKDNSPVVLACLITGYHPTSVTVTWYMG
TQSQPQRTFPEIQRRDSYYMTSSQLSTPLQQWRQGEYKCVVQHTA
SKSKKEIFRWPESPKAQASSVPTAQPQAEGSLAKATTAPATTRNT
GRGGEEKKKEKEKEEQEERETKTPECPSHTQPLGVYLLTPAVQDL
WLRDKATFTCFVVGSDLKDAHLTWEVAGKVPTGGVEEGLLERHSN
GSQSQHSRLTLPRSLWNAGTSVTCTLNHPSLPPQRLMALREPAAQ
APVKLSLNLLASSDPPEAASWLLCEVSGFSPPNILLMWLEDQREV
NTSGFAPARPPPQPGSTTFWAWSVLRVPAPPSPQPATYTCVVSHE
DSRTLLNASRSLEVSYVTDHGPMK
Human IgG1 constant region, Uniprot: P01857
(SEQ ID NO: 66)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQ
PENNYKTTPPVLDSDGSFFLYSKLTVDKSRWQQGNVFSCSVMHEA
LHNHYTQKSLSLSPGK
Human IgG2 constant region, Uniprot: P01859
(SEQ ID NO: 67)
ASTKGPSVFPLAPCSRSTSESTAALGCLVKDYFPEPVTVSWNSGA
LTSGVHTFPAVLQSSGLYSLSSVVTVPSSNFGTQTYTCNVDHKPS
NTKVDKTVERKCCVECPPCPAPPVAGPSVFLFPPKPKDTLMISRT
PEVTCVVVDVSHEDPEVQFNWYVDGVEVHNAKTKPREEQFNSTFR
VVSVLTVVHQDWLNGKEYKCKVSNKGLPAPIEKTISKTKGQPREP
QVYTLPPSREEMTKNQVSLTCLVKGFYPSDISVEWESNGQPENNY
KTTPPMLDSDGSFFLYSKLTVDKSRWQQGNVFSCSVMHEALHNHY
TQKSLSLSPGK
Human IgG3 constant region,
Uniprot: P01860
(SEQ ID NO: 68)
ASTKGPSVFPLAPCSRSTSGGTAALGCLVKDYFPEPVTVSWNSGA
LTSGVHTFPAVLQSSGLYSLSSVVTVPSSSLGTQTYTCNVNHKPS
NTKVDKRVELKTPLGDTTHTCPRCPEPKSCDTPPPCPRCPEPKSC
DTPPPCPRCPEPKSCDTPPPCPRCPAPELLGGPSVFLFPPKPKDT
LMISRTPEVTCVVVDVSHEDPEVQFKWYVDGVEVHNAKTKPREEQ
YNSTFRVVSVLTVLHQDWLNGKEYKCKVSNKALPAPIEKTISKTK
GQPREPQVYTLPPSREEMTKNQVSLTCLVKGFYPSDIAVEWESS
GQPENNYNTTPPMLDSDGSFFLYSKLTVDKSRWQQGNIFSCSVMH
EALHNRFTQKSLSLSPGK
Human IgM constant region, Uniprot: P01871
(SEQ ID NO: 69)
GSASAPTLFPLVSCENSPSDTSSVAVGCLAQDFLPDSITLSWKYK
NNSDISSTRGFPSVLRGGKYAATSQVLLPSKDVMQGTDEHVVCKV
QHPNGNKEKNVPLPVIAELPPKVSVFVPPRDGFFGNPRKSKLICQ
ATGFSPRQIQVSWLREGKQVGSGVTTDQVQAEAKESGPTTYKVTS
TLTIKESDWLGQSMFTCRVDHRGLTFQQNASSMCVPDQDTAIRVF
AIPPSFASIFLTKSTKLTCLVTDLTTYDSVTISWTRQNGEAVKTH
TNISESHPNATFSAVGEASICEDDWNSGERFTCTVTHTDLPSPLK
QTISRPKGVALHRPDVYLLPPAREQLNLRESATITCLVTGFSPAD
VFVQWMQRGQPLSPEKYVTSAPMPEPQAPGRYFAHSILTVSEEEW
NTGETYTCVAHEALPNRVTERTVDKSTGKPTLYNVSLVMSDTAGT
CY
Human IgG4 constant region, Uniprot: P01861
(SEQ ID NO: 70)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPSCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK
Human IgA1 constant region, Uniprot: P01876
(SEQ ID NO: 71)
ASPTSPKVFPLSLCSTQPDGNVVIACLVQGFFPQEPLSVTWSESG
QGVTARNFPPSQDASGDLYTTSSQLTLPATQCLAGKSVTCHVKHY
TNPSQDVTVPCPVPSTPPTPSPSTPPTPSPSCCHPRLSLHRPALE
DLLLGSEANLTCTLTGLRDASGVTFTWTPSSGKSAVQGPPERDLC
GCYSVSSVLPGCAEPWNHGKTFTCTAAYPESKTPLTATLSKSGNT
FRPEVHLLPPPSEELALNELVTLTCLARGFSPKDVLVRWLQGSQE
LPREKYLTWASRQEPSQGTTTFAVTSILRVAAEDWKKGDTFSCMV
GHEALPLAFTQKTIDRLAGKPTHVNVSVVMAEVDGTCY
Human IgA2 constant region,
Uniprot: P01877
(SEQ ID NO: 72)
ASPTSPKVFPLSLDSTPQDGNVVVACLVQGFFPQEPLSVTWSESG
QNVTARNFPPSQDASGDLYTTSSQLTLPATQCPDGKSVTCHVKHY
TNPSQDVTVPCPVPPPPPCCHPRLSLHRPALEDLLLGSEANLTCT
LTGLRDASGATFTWTPSSGKSAVQGPPERDLCGCYSVSSVLPGCA
QPWNHGETFTCTAAHPELKTPLTANITKSGNTFRPEVHLLPPPSE
ELALNELVTLTCLARGFSPKDVLVRWLQGSQELPREKYLTWASRQ
EPSQGTTTFAVTSILRVAAEDWKKGDTFSCMVGHEALPLAFTQKT
IDRMAGKPTHVNVSVVMAEVDGTCY
Human Ig kappa constant region,
Uniprot: P01834
(SEQ ID NO: 73)
TVAAPSVFIFPPSDEQLKSGTASVVCLLNNFYPREAKVQWKVDNA
LQSGNSQESVTEQDSKDSTYSLSSTLTLSKADYEKHKVYACEVTH
QGLSSPVTKSFNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 65-72. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 73.

In some embodiments, the immunoglobulin-related compositions of the present technology bind to the extracellular domain of a TSHR polypeptide. In certain embodiments, the epitope is a conformational epitope or non-conformational epitope. In some embodiments, the TSHR polypeptide has the amino acid sequence of SEQ ID NO: 74:

UniProtKB: P16473 (TSHR_HUMAN)
(SEQ ID NO: 74)
MRPADLLQLVLLLDLPRDLGGMGCSSPPCECHQEEDFRVTCKDIQ
RIPSLPPSTQTLKLIETHLRTIPSHAFSNLPNISRIYVSIDVTLQ
QLESHSFYNLSKVTHIEIRNTRNLTYIDPDALKELPLLKFLGIFN
TGLKMFPDLTKVYSTDIFFILEITDNPYMTSIPVNAFQGLCNETL
TLKLYNNGFTSVQGYAFNGTKLDAVYLNKNKYLTVIDKDAFGGVY
SGPSLLDVSQTSVTALPSKGLEHLKELIARNTWTLKKLPLSLSFL
HLTRADLSYPSHCCAFKNQKKIRGILESLMCNESSMQSLRQRKSV
NALNSPLHQEYEENLGDSIVGYKEKSKFQDTHNNAHYYVFFEEQE
DEIIGFGQELKNPQEETLQAFDSHYDYTICGDSEDMVCTPKSDEF
NPCEDIMGYKFLRIVVWFVSLLALLGNVFVLLILLTSHYKLNVPR
FLMCNLAFADFCMGMYLLLIASVDLYTHSEYYNHAIDWQTGPGCN
TAGFFTVFASELSVYTLTVITLERWYAITFAMRLDRKIRLRHACA
IMVGGWVCCFLLALLPLVGISSYAKVSICLPMDTETPLALAYIVF
VLTLNIVAFVIVCCCYVKIYITVRNPQYNPGDKDTKIAKRMAVLI
FTDFICMAPISFYALSAILNKPLITVSNSKILLVLFYPLNSCANP
FLYAIFTKAFQRDVFILLSKFGICKRQAQAYRGQRVPPKNSTDIQ
VQKVTHDMRQGLHNMEDVYELIENSHLTPKKQGQISEEYMQTVL

In any and all embodiments of the anti-TSHR multi-specific antibody disclosed herein, the antibody or antigen binding fragment binds to a TSHR polypeptide comprising amino acids 22-260 of SEQ ID NO: 74. The extracellular domain sequence of the TSHR polypeptide is:

(SEQ ID NO: 75)
MGCSSPPCECHQEEDFRVTCKDIQRIPSLPPSTQTLKLIETHLRT
IPSHAFSNLPNISRIYVSIDVTLQQLESHSFYNLSKVTHIEIRNT
RNLTYIDPDALKELPLLKFLGIFNTGLKMFPDLTKVYSTDIFFIL
EITDNPYMTSIPVNAFQGLCNETLTLKLYNNGFTSVQGYAFNGTK
LDAVYLNKNKYLTVIDKDAFGGVYSGPSLLDVSQTSVTALPSKGL
EHLKELIARNTWTL

Additionally or alternatively, in some embodiments, the multi-specific antibody or antigen binding fragment binds to the extracellular domain of a TSHR polypeptide.

In one aspect, the present disclosure provides a multi-specific immunoglobulin-related compositions (e.g., antibody or antigen binding fragment) comprising a TSHR antigen binding domain and a CD3 antigen binding domain, wherein (a) the TSHR antigen binding domain includes a heavy chain immunoglobulin variable domain (V_{H}) amino acid sequence that is at least 80%, at least 85%, at least 90%, at least 95%, or at least 99% identical to SEQ ID NO: 57 or SEQ ID NO: 59, and a light chain immunoglobulin variable domain (V_{L}) amino acid sequence that is at least 80%, at least 85%, at least 90%, at least 95%, or at least 99% identical to SEQ ID NO: 58 or SEQ ID NO: 60, and (b) the CD3 antigen binding domain includes a heavy chain immunoglobulin variable domain (V_{H}) amino acid sequence that is at least 80%, at least 85%, at least 90%, at least 95%, or at least 99% identical to SEQ ID NO: 61 or SEQ ID NO: 62 and a light chain immunoglobulin variable domain (V_{L}) amino acid sequence that is at least 80%, at least 85%, at least 90%, at least 95%, or at least 99% identical to SEQ ID NO: 63 or SEQ ID NO: 64. In another aspect, one or more amino acid residues in the immunoglobulin-related compositions provided herein are substituted with another amino acid. The substitution may be a “conservative substitution” as defined herein.

In another aspect, the present disclosure provides an isolated immunoglobulin-related composition (e.g., an antibody or antigen binding fragment thereof) comprising a heavy chain (HC) amino acid sequence comprising SEQ ID NO: 3, SEQ ID NO: 13, SEQ ID NO: 17, SEQ ID NO: 21, SEQ ID NO: 25, SEQ ID NO: 29, SEQ ID NO: 39, SEQ ID NO: 43, SEQ ID NO: 47, SEQ ID NO: 51, or a variant thereof having one or more conservative amino acid substitutions.

Additionally or alternatively, in some embodiments, the immunoglobulin-related compositions of the present technology comprise a light chain (LC) amino acid sequence comprising SEQ ID NO: 1, SEQ ID NO: 11, SEQ ID NO: 15, SEQ ID NO: 19, SEQ ID NO: 23, SEQ ID NO: 27, SEQ ID NO: 37, SEQ ID NO: 41, SEQ ID NO: 45, SEQ ID NO: 49, or a variant thereof having one or more conservative amino acid substitutions. In some embodiments, the immunoglobulin-related compositions of the present technology comprise a HC amino acid sequence and a LC amino acid sequence selected from the group consisting of SEQ ID NO: 3 and SEQ ID NO: 1, and SEQ ID NO: 29 and SEQ ID NO: 27, respectively.

In any of the above embodiments of the immunoglobulin-related compositions, the HC and LC immunoglobulin variable domain sequences form an antigen binding site that binds to the extracellular domain of a TSHR polypeptide. In certain embodiments, the extracellular domain comprises the amino acids at positions 22-260 of SEQ ID NO: 74. In some embodiments, the epitope is a conformational epitope or a non-conformational epitope.

In some embodiments, the HC and LC immunoglobulin variable domain sequences are components of the same polypeptide chain. In other embodiments, the HC and LC immunoglobulin variable domain sequences are components of different polypeptide chains. In certain embodiments, the antibody is a full-length antibody.

In one aspect, the present disclosure provides an immunoglobulin-related composition comprising an amino acid sequence that is at least 80%, at least 85%, at least 90%, at least 95%, or at least 99% identical to an amino acid sequence selected from SEQ ID NOs: 5, 7, 9, 31, 33, and 35. In certain embodiments, the immunoglobulin-related composition comprises an amino acid sequence selected from any one of SEQ ID NOs: 5, 7, 9, 31, 33, and 35.

In another aspect, the present disclosure provides an antibody comprising (a) a LC sequence that is at least 80%, at least 85%, at least 90%, at least 95%, or at least 99% identical to the LC sequence present in SEQ ID NO: 1, SEQ ID NO: 11, SEQ ID NO: 15, SEQ ID NO: 19, SEQ ID NO: 23, SEQ ID NO: 27, SEQ ID NO: 37, SEQ ID NO: 41, SEQ ID NO: 45, or SEQ ID NO: 49; and/or (b) a HC sequence that is at least 80%, at least 85%, at least 90%, at least 95%, or at least 99% identical to the HC sequence present in SEQ ID NO: 3, SEQ ID NO: 13, SEQ ID NO: 17, SEQ ID NO: 21, SEQ ID NO: 25, SEQ ID NO: 29, SEQ ID NO: 39, SEQ ID NO: 43, SEQ ID NO: 47, or SEQ ID NO: 51.

In one aspect, the present disclosure provides a multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain are covalently bonded to one another, and wherein: (a) each of the first polypeptide chain and the fourth polypeptide chain comprises in the N-terminal to C-terminal direction: (i) a light chain variable domain of a first immunoglobulin that is capable of specifically binding to a first epitope; (ii) a light chain constant domain of the first immunoglobulin; (iii) a flexible peptide linker comprising the amino acid sequence (GGGGS)₃; and (iv) a light chain variable domain of a second immunoglobulin that is linked to a complementary heavy chain variable domain of the second immunoglobulin, or a heavy chain variable domain of a second immunoglobulin that is linked to a complementary light chain variable domain of the second immunoglobulin, wherein the light chain and heavy chain variable domains of the second immunoglobulin are capable of specifically binding to a second epitope, and are linked together via a flexible peptide linker comprising the amino acid sequence (GGGGS)₆ to form a single-chain variable fragment; and (b) each of the second polypeptide chain and the third polypeptide chain comprises in the N-terminal to C-terminal direction: (i) a heavy chain variable domain of the first immunoglobulin that is capable of specifically binding to the first epitope; and (ii) a heavy chain constant domain of the first immunoglobulin; and wherein the heavy chain variable domain of the first immunoglobulin is SEQ ID NO: 57 or SEQ ID NO: 59, wherein the light chain variable domain of the first immunoglobulin is SEQ ID NO: 58 or SEQ ID NO: 60, wherein the heavy chain variable domain of the second immunoglobulin is SEQ ID NO: 61 or SEQ ID NO: 62 and wherein the light chain variable domain of the second immunoglobulin is SEQ ID NO: 63 or SEQ ID NO: 64.

In one aspect, the present disclosure provides a multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain are covalently bonded to one another, and wherein: (a) each of the first polypeptide chain and the fourth polypeptide chain comprises in the N-terminal to C-terminal direction: (i) a light chain variable domain of a first immunoglobulin that is capable of specifically binding to a first epitope; (ii) a light chain constant domain of the first immunoglobulin; (iii) a flexible peptide linker comprising the amino acid sequence (GGGGS)₃; and (iv) a light chain variable domain of a second immunoglobulin that is linked to a complementary heavy chain variable domain of the second immunoglobulin, or a heavy chain variable domain of a second immunoglobulin that is linked to a complementary light chain variable domain of the second immunoglobulin, wherein the light chain and heavy chain variable domains of the second immunoglobulin are capable of specifically binding to a second epitope, and are linked together via a flexible peptide linker comprising the amino acid sequence (GGGGS)₆ to form a single-chain variable fragment; and (b) each of the second polypeptide chain and the third polypeptide chain comprises in the N-terminal to C-terminal direction: (i) a heavy chain variable domain of the first immunoglobulin that is capable of specifically binding to the first epitope; and (ii) a heavy chain constant domain of the first immunoglobulin; and wherein the heavy chain variable domain of the second immunoglobulin is SEQ ID NO: 57 or SEQ ID NO: 59, wherein the light chain variable domain of the second immunoglobulin is SEQ ID NO: 58 or SEQ ID NO: 60, wherein the heavy chain variable domain of the first immunoglobulin is SEQ ID NO: 61 or SEQ ID NO: 62 and wherein the light chain variable domain of the first immunoglobulin is SEQ ID NO: 63 or SEQ ID NO: 64.

In another aspect, the present disclosure provides a heterodimeric multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃; and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ to form a single-chain variable fragment; (b) the second polypeptide chain comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide chain comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide chain comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃; and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ to form a single-chain variable fragment; wherein VL-1 or VL-3 comprises a V_{L} amino acid sequence selected from any one of SEQ ID NO: 58 or SEQ ID NO: 60, wherein VH-1 or VH-3 comprises a V_{H} amino acid sequence selected from any one of SEQ ID NO: 57 or SEQ ID NO: 59, wherein VH-2 or VH-4 comprises a V_{H} amino acid sequence selected from any one of SEQ ID NO: 61 or SEQ ID NO: 62, and wherein VL-2 or VL-4 comprises a V_{L} amino acid sequence selected from any one of SEQ ID NO: 63 or SEQ ID NO: 64.

In another aspect, the present disclosure provides a heterodimeric multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃; and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ to form a single-chain variable fragment; (b) the second polypeptide chain comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide chain comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide chain comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃; and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ to form a single-chain variable fragment; wherein VL-2 or VL-4 comprises a V_{L} amino acid sequence selected from any one of SEQ ID NO: 58 or SEQ ID NO: 60, wherein VH-2 or VH-4 comprises a V_{H} amino acid sequence selected from any one of SEQ ID NO: 57 or SEQ ID NO: 59, wherein VH-1 or VH-3 comprises a V_{H} amino acid sequence selected from any one of SEQ ID NO: 61 or SEQ ID NO: 62, and wherein VL-1 or VL-3 comprises a V_{L} amino acid sequence selected from any one of SEQ ID NO: 63 or SEQ ID NO: 64.

In some embodiments, the immunoglobulin-related compositions of the present technology bind specifically to at least one TSHR polypeptide. In some embodiments, the immunoglobulin-related compositions of the present technology bind at least one TSHR polypeptide with a dissociation constant (K_{D}) of about 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the immunoglobulin-related compositions are monoclonal antibodies, chimeric antibodies, humanized antibodies or multi-specific antibodies. In some embodiments, the antibodies comprise a human antibody framework region.

In any and all embodiments of the multi-specific antibodies disclosed herein, the multi-specific antibodies bind to one or more of TSHR, CD3, CD4, CD8, CD20, CD19, CD21, CD23, CD46, CD80, HLA-DR, CD74, CD22, CD14, CD15, CD16, CD123, TCR gamma/delta, NKp46, KIR, PD-1, PD-L1, CD28, B7H3, HER2, DOTA (metal) complex, benzyl-DOTA (metal) complex, or a small molecule DOTA hapten. In some embodiments of the multi-specific antibody or multi-specific antigen binding fragment described herein, the antibody or antigen binding fragment comprises a catalytic antibody, an immune checkpoint inhibitor, or an immune checkpoint activator.

In certain embodiments, the immunoglobulin-related compositions contain an IgG1 constant region comprising one or more amino acid substitutions selected from the group consisting of N297A and K322A. Additionally or alternatively, in some embodiments, the immunoglobulin-related compositions contain an IgG4 constant region comprising a S228P mutation. In any of the above embodiments, the antibody is a chimeric antibody, a humanized antibody, or a bispecific antibody.

In some aspects, the anti-TSHR immunoglobulin-related compositions described herein contain structural modifications to facilitate rapid binding and cell uptake and/or slow release. In some aspects, the anti-TSHR immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

In one aspect, the present technology provides a nucleic acid sequence encoding any of the immunoglobulin-related compositions described herein. Also disclosed herein are recombinant nucleic acid sequences encoding any of the antibodies described herein. In some embodiments, the recombinant nucleic acid sequences may be one or more nucleic acid sequences selected from the group consisting of SEQ ID NOs: 2, 4, 28 and 30. In another aspect, the present technology provides a host cell expressing any nucleic acid sequence encoding any of the immunoglobulin-related compositions described herein.

In another aspect, the present technology provides a cell (e.g., an immune cell, such as a T cell) that is coated with any and all embodiments of the multi-specific antibody disclosed herein.

The immunoglobulin-related compositions of the present technology (e.g., an anti-TSHR antibody) can be monospecific, bispecific, trispecific or of greater multi-specificity. Multi-specific antibodies can be specific for different epitopes of one or more TSHR polypeptides or can be specific for both the TSHR polypeptide(s) as well as for heterologous compositions, such as a heterologous polypeptide or solid support material. See, e.g., WO 93/17715; WO 92/08802; WO 91/00360; WO 92/05793; Tutt et al., J. Immunol. 147:60-69 (1991); U.S. Pat. Nos. 5,573,920, 4,474,893, 5,601,819, 4,714,681, 4,925,648; 6,106,835; Kostelny et al., J. Immunol. 148:1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis [succinimidylsuccinate]), DSS (i.e., disuccinimidyl suberate), DMA (i.e., dimethyl adipimidate.2HCl), DTSSP (i.e., 3,3′-dithiobis [sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido) hexanoate), LC-SPDP (i.e., succinimidyl 6-(3-[2-pyridyldithio]-propionamido) hexanoate), Sulfo-LC-SPDP (i.e., sulfosuccinimidyl 6-(3-[2-pyridyldithio]-propionamido) hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

### A. Methods of Preparing Anti-TSHR Multi-Specific Antibodies of the Present Technology

Overview. Initially, a target polypeptide is chosen to which an antibody of the present technology can be raised. For example, an antibody may be raised against the full-length TSHR protein, or to a portion of the extracellular domain of the TSHR protein. Techniques for generating antibodies directed to such target polypeptides are well known to those skilled in the art. Examples of such techniques include, for example, but are not limited to, those involving display libraries, xeno or human mice, hybridomas, and the like. Target polypeptides within the scope of the present technology include any polypeptide derived from TSHR protein containing the extracellular domain which is capable of eliciting an immune response. In certain embodiments, the extracellular domain comprises the amino acids at positions 22-260 of SEQ ID NO: 74.

It should be understood that recombinantly engineered antibodies and antibody fragments, e.g., antibody-related polypeptides, which are directed to TSHR protein and fragments thereof are suitable for use in accordance with the present disclosure.

Anti-TSHR multi-specific antibodies that can be subjected to the techniques set forth herein include monoclonal and polyclonal antibodies, and antibody fragments such as Fab, Fab′, F(ab′)₂, Fd, scFv, diabodies, antibody light chains, antibody heavy chains and/or antibody fragments. Methods useful for the high yield production of antibody Fv-containing polypeptides, e.g., Fab′ and F(ab′)₂ antibody fragments have been described. See U.S. Pat. No. 5,648,237.

Generally, an antibody is obtained from an originating species. More particularly, the nucleic acid or amino acid sequence of the variable portion of the light chain, heavy chain or both, of an originating species antibody having specificity for a target polypeptide antigen is obtained. An originating species is any species which was useful to generate the antibody of the present technology or library of antibodies, e.g., rat, mouse, rabbit, chicken, monkey, human, and the like.

Phage or phagemid display technologies are useful techniques to derive the antibodies of the present technology. Techniques for generating and cloning monoclonal antibodies are well known to those skilled in the art. Expression of sequences encoding antibodies of the present technology, can be carried out in E. coli.

Due to the degeneracy of nucleic acid coding sequences, other sequences which encode substantially the same amino acid sequences as those of the naturally occurring proteins may be used in the practice of the present technology These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobulin according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant forms an operative antibody which recognizes TSHR proteins. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosylation, proteolytic cleavage, linkage to an antibody molecule or other cellular ligands, etc. Additionally, an immunoglobulin encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Preparation of Polyclonal Antisera and Immunogens. Methods of generating antibodies or antibody fragments of the present technology typically include immunizing a subject (generally a non-human subject such as a mouse or rabbit) with a purified TSHR protein or fragment thereof, or with a cell expressing the TSHR protein or fragment thereof. An appropriate immunogenic preparation can contain, e.g., a recombinantly-expressed TSHR protein or a chemically-synthesized TSHR peptide. The extracellular domain of the TSHR protein, or a portion or fragment thereof, can be used as an immunogen to generate an anti-TSHR multi-specific antibody that binds to the TSHR protein, or a portion or fragment thereof using standard techniques for polyclonal and monoclonal antibody preparation. In certain embodiments, the extracellular domain comprises the amino acids at positions 22-260 of SEQ ID NO: 74. In some embodiments, the antigenic TSHR peptide comprises at least 10, at least 20, at least 30, at least 40, at least 50, at least 60, at least 70, at least 80, at least 90, or at least 100 amino acid residues. Longer antigenic peptides are sometimes desirable over shorter antigenic peptides, depending on use and according to methods well known to those skilled in the art. Multimers of a given epitope are sometimes more effective than a monomer.

An appropriate immunogenic preparation can contain, e.g., a recombinantly-expressed TSHR protein or a chemically-synthesized TSHR polypeptide comprising amino acid sequence of SEQ ID NO: 75. The extracellular domain of the TSHR protein, or a portion or fragment thereof, can be used as an immunogen to generate an anti-TSHR multi-specific antibody that binds to the extracellular domain of the TSHR protein.

If needed, the immunogenicity of the TSHR protein (or fragment thereof) can be increased by fusion or conjugation to a carrier protein such as keyhole limpet hemocyanin (KLH) or ovalbumin (OVA). Many such carrier proteins are known in the art. Synthetic dendromeric trees can be added to reactive amino acid side chains, e.g., lysine, to enhance the immunogenic properties of TSHR protein. Also, CPG-dinucleotide motifs can be added to enhance the immunogenic properties of the TSHR protein. One can also combine the TSHR protein with a conventional adjuvant such as Freund's complete or incomplete adjuvant to increase the subject's immune reaction to the polypeptide. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. These techniques are standard in the art.

Alternatively, nanoparticles, for example, virus-like particles (VLPs), can be used to present antigens, e.g., TSHR protein, to a host animal. Virus-like particles are multiprotein structures that mimic the organization and conformation of authentic native viruses without being infectious, since they do not carry any viral genetic material (Urakami A, et al, Clin Vaccine Immunol 24: e00090-17 (2017)). When introduced to a host immune system, VLPs can evoke effective immune responses, making them attractive carriers of foreign antigens. An important advantage of a VLP-based antigen presenting platform is that it can display antigens in a dense, repetitive manner. Thus, antigen-bearing VLPs are able to induce strong B-cell responses by effectively enabling the cross-linking of B cell receptors (BCRs). VLPs may be genetically manipulated to fine their properties, e.g., immunogenicity. These techniques are standard in the art.

The isolation of sufficient purified protein or polypeptide to which an antibody is to be raised may be time consuming and sometimes technically challenging. Additional challenges associated with conventional protein-based immunization include concerns over safety, stability, scalability and consistency of the protein antigen. Nucleic acid (DNA and RNA) based immunizations have emerged as promising alternatives. DNA vaccines are usually based on bacterial plasmids that encode the polypeptide sequence of candidate antigen, e.g., TSHR. With a robust eukaryotic promoter, the encoded antigen can be expressed to yield enough levels of transgene expression once the host is inoculated with the plasmids (Galvin T. A., et al., Vaccine 2000, 18:2566-2583). Modern DNA vaccine generation relies on DNA synthesis, possibly one-step cloning into the plasmid vector and subsequent isolation of the plasmid, which takes significantly less time and cost to manufacture. The resulting plasmid DNA is also highly stable at room temperature, avoiding cold transportation and leading to substantially extended shelf-life. These techniques are standard in the art.

Alternatively, nucleic acid sequences encoding the antigen of interest, e.g., TSHR, can be synthetically introduced into a mRNA molecule. The mRNA is then delivered into a host animal, whose cells would recognize and translate the mRNA sequence to the polypeptide sequence of the candidate antigen, e.g., TSHR, thus triggering the immune response to the foreign antigen. An attractive feature of mRNA antigen or mRNA vaccine is that mRNA is a non-infectious, non-integrating platform. There is no potential risk of infection or insertional mutagenesis associated with DNA vaccines. In addition, mRNA is degraded by normal cellular processes and has a controllable in vivo half-life through modification of design and delivery methods (Kariko, K., et al., Mol Ther 16:1833-1840 (2008), Kauffman, K. J., et al., J Control Release 240, 227-234 (2016); Guan, S. & Rosenecker, J., Gene Ther 24, 133-143 (2017); Thess, A., et al., Mol Ther 23, 1456-1464 (2015)). These techniques are standard in the art.

In describing the present technology, immune responses may be described as either “primary” or “secondary” immune responses. A primary immune response, which is also described as a “protective” immune response, refers to an immune response produced in an individual as a result of some initial exposure (e.g., the initial “immunization” or “priming”) to a particular antigen, e.g., TSHR protein. In some embodiments, the immunization can occur as a result of vaccinating the individual with a vaccine containing the antigen. For example, the vaccine can be a TSHR vaccine comprising one or more TSHR protein-derived antigens. A primary immune response can become weakened or attenuated over time and can even disappear or at least become so attenuated that it cannot be detected. Accordingly, the present technology also relates to a “secondary” immune response, which is also described here as a “memory immune response.” The term secondary immune response refers to an immune response elicited in an individual after a primary immune response has already been produced.

Thus, a secondary immune response can be elicited, e.g., to enhance an existing immune response that has become weakened or attenuated, or to recreate a previous immune response that has either disappeared or can no longer be detected (e.g., “boosting”). The secondary or memory immune response can be either a humoral (antibody) response or a cellular response. A secondary or memory humoral response occurs upon stimulation of memory B cells that were generated at the first presentation of the antigen. Delayed type hypersensitivity (DTH) reactions are a type of cellular secondary or memory immune response that are mediated by CD4⁺ T cells. A first exposure to an antigen primes the immune system and additional exposure(s) results in a DTH.

Following appropriate immunization, the anti-TSHR multi-specific antibody can be prepared from the subject's serum. If desired, the antibody molecules directed against the TSHR protein can be isolated from the mammal (e.g., from the blood) and further purified by well-known techniques, such as polypeptide A chromatography to obtain the IgG fraction.

Monoclonal Antibody. In one embodiment of the present technology, the antibody is an anti-TSHR monoclonal multi-specific antibody. For example, in some embodiments, the anti-TSHR monoclonal multi-specific antibody may be a human or a mouse anti-TSHR monoclonal multi-specific antibody. For preparation of monoclonal antibodies directed towards the TSHR protein, or derivatives (e.g., the anti-TSHR multi-specific antibodies of the present technology), fragments, analogs or homologs thereof, any technique that provides for the production of antibody molecules by continuous cell line culture can be utilized. Such techniques include, but are not limited to, the hybridoma technique (See, e.g., Kohler & Milstein, 1975. Nature 256:495-497); the trioma technique; the human B-cell hybridoma technique (See, e.g., Kozbor, et al., 1983. Immunol. Today 4:72) and the EBV hybridoma technique to produce human monoclonal antibodies (See, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (See, e.g., Cote, et al., 1983. Proc Natl Acad Sci USA 80:2026-2030) or by transforming human B-cells with Epstein Barr Virus in vitro (See, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then DNAs encoding antibodies or fragments thereof, such as variable domains, are reconstructed from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the TSHR protein. Alternatively, hybridomas expressing anti-TSHR monoclonal antibodies of the present technology can be prepared by immunizing a subject and then isolating hybridomas from the subject's spleen using routine methods. See, e.g., Milstein et al., (Galfre and Milstein, Methods Enzymol (1981) 73:3-46). Screening the hybridomas using standard methods will produce monoclonal antibodies of varying specificity (i.e., for different epitopes) and affinity. A selected monoclonal antibody with the desired properties, e.g., TSHR binding, can be used as expressed by the hybridoma; it can be bound to a molecule such as polyethylene glycol (PEG) to alter its properties, or a cDNA encoding it can be isolated, sequenced and manipulated in various ways. Other manipulations include substituting or deleting particular amino acyl residues that contribute to instability of the antibody during storage or after administration to a subject, and affinity maturation techniques to improve affinity of the antibody of the TSHR protein.

Hybridoma Technique. In some embodiments, the antibody of the present technology is an anti-TSHR monoclonal multi-specific antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, NY, 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique. As noted above, the antibodies of the present technology can be produced through the application of recombinant DNA and phage display technology. For example, anti-TSHR multi-specific antibodies, can be prepared using various phage display methods known in the art. In phage display methods, functional antibody domains are displayed on the surface of a phage particle which carries polynucleotide sequences encoding them. Phages with a desired binding property are selected from a repertoire or combinatorial antibody library (e.g., human or murine) by selecting directly with an antigen, typically an antigen bound or captured to a solid surface or bead. Phages used in these methods are typically filamentous phage including fd and M13 with Fab, Fv or disulfide stabilized Fv antibody domains that are recombinantly fused to either the phage gene III or gene VIII protein. In addition, methods can be adapted for the construction of Fab expression libraries (See, e.g., Huse, et al., Science 246:1275-1281, 1989) to allow rapid and effective identification of monoclonal Fab fragments with the desired specificity for a TSHR polypeptide, e.g., a polypeptide or derivatives, fragments, analogs or homologs thereof. Other examples of phage display methods that can be used to make the antibodies of the present technology include those disclosed in Huston et al., Proc. Natl. Acad. Sci U.S.A., 85:5879-5883, 1988; Chaudhary et al., Proc. Natl. Acad. Sci U.S.A., 87:1066-1070, 1990; Brinkman et al., J. Immunol. Methods 182:41-50, 1995; Ames et al., J. Immunol. Methods 184:177-186, 1995; Kettleborough et al., Eur. J. Immunol. 24:952-958, 1994; Persic et al., Gene 187:9-18, 1997; Burton et al., Advances in Immunology 57:191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12:864-869, 1992; and Sawai et al., AJRI 34:26-34, 1995; and Better et al., Science 240:1041-1043, 1988.

Generally, hybrid antibodies or hybrid antibody fragments that are cloned into a display vector can be selected against the appropriate antigen in order to identify variants that maintain good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Expression of Recombinant Anti-TSHR multi-specific antibodies. As noted above, the antibodies of the present technology can be produced through the application of recombinant DNA technology. Recombinant polynucleotide constructs encoding an anti-TSHR multi-specific antibody of the present technology typically include an expression control sequence operably-linked to the coding sequences of the antibody chains, including naturally-associated or heterologous promoter regions. As such, another aspect of the technology includes vectors containing one or more nucleic acid sequences encoding an anti-TSHR multi-specific antibody of the present technology. For recombinant expression of one or more of the polypeptides of the present technology, the nucleic acid containing all or a portion of the nucleotide sequence encoding the anti-TSHR multi-specific antibody of the present technology is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the anti-TSHR multi-specific antibody of the present technology, and the collection and purification of the anti-TSHR multi-specific antibodies of the present technology. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein with TSHR binding properties in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., anti-TSHR multi-specific antibody), include but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding an anti-TSHR multi-specific antibody of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., anti-TSHR multi-specific antibody, etc.).

Another aspect of the present technology pertains to anti-TSHR multi-specific antibody-expressing host cells, which contain a nucleic acid encoding one or more anti-TSHR multi-specific antibodies. The recombinant expression vectors of the present technology can be designed for expression of an anti-TSHR multi-specific antibody in prokaryotic or eukaryotic cells. For example, an anti-TSHR multi-specific antibody can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., anti-TSHR multi-specific antibody, via expression of stochastically generated polynucleotide sequences has been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., an anti-TSHR multi-specific antibody, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl Acids Res 20: 2111-2118). Such alteration of nucleic acid sequences of the present technology can be carried out by standard DNA synthesis techniques.

In another embodiment, the anti-TSHR multi-specific antibody expression vector is a yeast expression vector. Examples of vectors for expression in yeast Saccharomyces cerevisiae include pYepSec1 (Baldari, et al., 1987. EMBO J. 6:229-234), pMFa (Kurjan and Herskowitz, Cell 30:933-943, 1982), pJRY88 (Schultz et al., Gene 54:113-123, 1987), pYES2 (Invitrogen Corporation, San Diego, Calif.), and picZ (Invitrogen Corp, San Diego, Calif.). Alternatively, an anti-TSHR multi-specific antibody can be expressed in insect cells using baculovirus expression vectors. Baculovirus vectors available for expression of polypeptides, e.g., an anti-TSHR multi-specific antibody, in cultured insect cells (e.g., SF9 cells) include the pAc series (Smith, et al., Mol Cell Biol 3:2156-2165, 1983) and the pVL series (Lucklow and Summers, 1989. Virology 170:31-39).

In yet another embodiment, a nucleic acid encoding an anti-TSHR multi-specific antibody of the present technology is expressed in mammalian cells using a mammalian expression vector. Examples of mammalian expression vectors include, e.g., but are not limited to, pCDM8 (Seed, Nature 329:840, 1987) and pMT2PC (Kaufman, et al., EMBO J. 6:187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the anti-TSHR multi-specific antibody of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N. Y., 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1:268-277, 1987), lymphoid-specific promoters (Calame and Eaton, Adv. Immunol. 43:235-275, 1988), promoters of T cell receptors (Winoto and Baltimore, EMBO J. 8:729-733, 1989) and immunoglobulins (Banerji, et al., 1983. Cell 33:729-740; Queen and Baltimore, Cell 33:741-748, 1983), neuron-specific promoters (e.g., the neurofilament promoter; Byrne and Ruddle, Proc Natl Acad Sci USA 86:5473-5477, 1989), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249:374-379, 1990) and the α-fetoprotein promoter (Campes and Tilghman, Genes Dev. 3:537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an anti-TSHR multi-specific antibody can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, NY, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89:49, 1986. Illustrative expression control sequences are promoters derived from endogenous genes, cytomegalovirus, SV40, adenovirus, bovine papillomavirus, and the like. Co et al., J Immunol. 148:1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well-known methods, depending on the type of cellular host.

Non-limiting examples of suitable vectors include those designed for propagation and expansion, or for expression or both. For example, a cloning vector can be selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as lamda-GT10, lamda-GT11, lamda-ZapII (Stratagene), lamda-EMBL4, and lamda-NM1149, can also be used. Non-limiting examples of plant expression vectors include pBI110, pBI101.2, pBI101.3, pBI121 and pBIN19 (Clontech). Non-limiting examples of animal expression vectors include pEUK-C1, pMAM and pMAMneo (Clontech). The TOPO cloning system (Invitrogen, Calsbad, CA, Carlsbad, CA) can also be used in accordance with the manufacturer's recommendations.

In certain embodiments, the vector is a mammalian vector. In certain embodiments, the mammalian vector contains at least one promoter element, which mediates the initiation of transcription of mRNA, the antibody-coding sequence, and signals required for the termination of transcription and polyadenylation of the transcript. In certain embodiments, the mammalian vector contains additional elements, such as, for example, enhancers, Kozak sequences and intervening sequences flanked by donor and acceptor sites for RNA splicing. In certain embodiments, highly efficient transcription can be achieved with, for example, the early and late promoters from SV40, the long terminal repeats (LTRS) from retroviruses, for example, RSV, HTLVI, HIVI and the early promoter of the cytomegalovirus (CMV). Cellular elements can also be used (e.g., the human actin promoter). Non-limiting examples of mammalian expression vectors include, vectors such as pIRESIneo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen, Calsbad, CA), PSVL and PMSG (Pharmacia, Uppsala, Sweden), pRSVcat (ATCC 37152), pSV2dhfr (ATCC 37146) and pBC12MI (ATCC 67109). Non-limiting examples of mammalian host cells that can be used in combination with such mammalian vectors include human Hela 293, HEK 293, H9 and Jurkat cells, mouse 3T3, NIH3T3 and C127 cells, Cos 1, Cos 7 and CV 1, quail QC1-3 cells, mouse L cells and Chinese hamster ovary (CHO) cells.

In certain embodiments, the vector is a viral vector, for example, retroviral vectors, parvovirus-based vectors, e.g., adeno-associated virus (AAV)-based vectors, AAV-adenoviral chimeric vectors, and adenovirus-based vectors, and lentiviral vectors, such as Herpes simplex (HSV)-based vectors. In certain embodiments, the viral vector is manipulated to render the virus replication deficient. In certain embodiments, the viral vector is manipulated to eliminate toxicity to the host. These viral vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994).

In certain embodiments, a vector or polynucleotide described herein can be transferred to a cell (e.g., an ex vivo cell) by conventional techniques and the resulting cell can be cultured by conventional techniques to produce an anti-TSHR multi-specific antibody or antigen binding fragment described herein. Accordingly, provided herein are cells comprising a polynucleotide encoding an anti-TSHR multi-specific antibody or antigen binding fragment thereof operably linked to a regulatory expression element (e.g., promoter) for expression of such sequences in the host cell. In certain embodiments, a vector encoding the heavy chain operably linked to a promoter and a vector encoding the light chain operably linked to a promoter can be co-expressed in the cell for expression of the entire anti-TSHR multi-specific antibody or antigen binding fragment. In certain embodiments, a cell comprises a vector comprising a polynucleotide encoding both the heavy chain and the light chain of an anti-TSHR multi-specific antibody or antigen binding fragment described herein that are operably linked to a promoter. In certain embodiments, a cell comprises two different vectors, a first vector comprising a polynucleotide encoding a heavy chain operably linked to a promoter, and a second vector comprising a polynucleotide encoding a light chain operably linked to a promoter. In certain embodiments, a first cell comprises a first vector comprising a polynucleotide encoding a heavy chain of an anti-TSHR multi-specific antibody or antigen binding fragment described herein, and a second cell comprises a second vector comprising a polynucleotide encoding a light chain of an anti-TSHR multi-specific antibody or antigen binding fragment described herein. In certain embodiments, provided herein is a mixture of cells comprising said first cell and said second cell. Examples of cells include, but are not limited to, a human cell, a human cell line, E. coli (e.g., E. coli TB-1, TG-2, DH5a, XL-Blue MRF′ (Stratagene), SA2821 and Y1090), B. subtilis, P. aerugenosa, S. cerevisiae, N. crassa, insect cells (e.g., Sf9, Ea4) and the like.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the anti-TSHR multi-specific antibody or can be introduced on a separate vector. Cells stably transfected with the introduced nucleic acid can be identified by drug selection (e.g., cells that have incorporated the selectable marker gene will survive, while the other cells die).

A host cell that includes an anti-TSHR multi-specific antibody of the present technology, such as a prokaryotic or eukaryotic host cell in culture, can be used to produce (i.e., express) recombinant anti-TSHR multi-specific antibody. In one embodiment, the method comprises culturing the host cell (into which a recombinant expression vector encoding the anti-TSHR multi-specific antibody has been introduced) in a suitable medium such that the anti-TSHR multi-specific antibody is produced. In another embodiment, the method further comprises the step of isolating the anti-TSHR multi-specific antibody from the medium or the host cell. Once expressed, collections of the anti-TSHR multi-specific antibody, e.g., the anti-TSHR multi-specific antibodies or the anti-TSHR multi-specific antibody-related polypeptides are purified from culture media and host cells. The anti-TSHR multi-specific antibody can be purified according to standard procedures of the art, including HPLC purification, column chromatography, gel electrophoresis and the like. In one embodiment, the anti-TSHR multi-specific antibody is produced in a host organism by the method of Boss et al., U.S. Pat. No. 4,816,397. Usually, anti-TSHR multi-specific antibody chains are expressed with signal sequences and are thus released to the culture media. However, if the anti-TSHR multi-specific antibody chains are not naturally secreted by host cells, the anti-TSHR multi-specific antibody chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y., 1982).

Polynucleotides encoding anti-TSHR multi-specific antibodies, e.g., the anti-TSHR multi-specific antibody coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Single-Chain Antibodies. In one embodiment, the anti-TSHR multi-specific antibody of the present technology is a single-chain anti-TSHR multi-specific antibody. According to the present technology, techniques can be adapted for the production of single-chain antibodies specific to a TSHR protein (See, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203:46-88, 1991; Shu, L. et al., Proc Natl Acad Sci USA, 90:7995-7999, 1993; and Skerra et al., Science 240:1038-1040, 1988.

Chimeric and Humanized Antibodies. In one embodiment, the anti-TSHR multi-specific antibody of the present technology is a chimeric anti-TSHR multi-specific antibody. In one embodiment, the anti-TSHR multi-specific antibody of the present technology is a humanized anti-TSHR multi-specific antibody. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant anti-TSHR multi-specific antibodies, such as chimeric and humanized monoclonal antibodies, comprising both human and non-human portions, can be made using standard recombinant DNA techniques, and are within the scope of the present technology. For some uses, including in vivo use of the anti-TSHR multi-specific antibody of the present technology in humans as well as use of these agents in in vitro detection assays, it is possible to use chimeric, humanized, or bispecific antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240:1041-1043; Liu, et al., 1987. Proc Natl Acad Sci USA 84:3439-3443; Liu, et al., 1987. J. Immunol. 139:3521-3526; Sun, et al., 1987. Proc Natl Acad Sci USA 84:214-218; Nishimura, et al., 1987. Cancer Res. 47:999-1005; Wood, et al., 1985. Nature 314:446-449; Shaw, et al., 1988. J. Natl. Cancer Inst. 80:1553-1559; Morrison (1985) Science 229:1202-1207; Oi, et al. (1986) BioTechniques 4:214; Jones, et al., 1986. Nature 321:552-525; Verhoeyan, et al., 1988. Science 239:1534; Morrison, Science 229: 1202, 1985; Oi et al., BioTechniques 4:214, 1986; Gillies et al., J. Immunol. Methods, 125:191-202, 1989; U.S. Pat. No. 5,807,715; and Beidler, et al., 1988. J. Immunol. 141:4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7:805-814, 1994; Roguska et al., PNAS 91:969-973, 1994), and chain shuffling (U.S. Pat. No. 5,565,332). In one embodiment, a cDNA encoding a murine anti-TSHR multi-specific antibody is digested with a restriction enzyme selected specifically to remove the sequence encoding the Fc constant region, and the equivalent portion of a cDNA encoding a human Fc constant region is substituted (See Robinson et al., PCT/US86/02269; Akira et al., European Patent Application 184, 187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240:1041-1043; Liu et al. (1987) Proc Natl Acad Sci USA 84:3439-3443; Liu et al. (1987) J Immunol 139:3521-3526; Sun et al. (1987) Proc Natl Acad Sci USA 84:214-218; Nishimura et al. (1987) Cancer Res 47:999-1005; Wood et al. (1985) Nature 314:446-449; and Shaw et al. (1988) J. Natl. Cancer Inst. 80:1553-1559; U.S. Pat. Nos. 6,180,370; 6,300,064; 6,696,248; 6,706,484; 6,828,422.

In one embodiment, the present technology provides the construction of humanized anti-TSHR multi-specific antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject. In one embodiment, the present technology provides for humanized anti-TSHR multi-specific antibodies, heavy and light chain immunoglobulins.

CDR-Grafted Antibodies. In some embodiments, the anti-TSHR multi-specific antibody of the present technology is an anti-TSHR CDR-grafted antibody. Generally the donor and acceptor antibodies used to generate the anti-TSHR CDR-grafted antibody are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one needs to replace only as many as necessary to permit adequate binding of the resulting CDR-grafted antibody to TSHR protein. Methods for generating CDR-grafted and humanized antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545, 142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting anti-TSHR CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified anti-TSHR CDR-grafted antibody significantly compared to the same antibody with a fully human FR.

Multi-specific Antibodies. A multi-specific antibody is an antibody that can bind simultaneously to multiple targets that have a distinct structure, e.g., two or more different target antigens, two or more different epitopes on the same target antigen, or a hapten and a target antigen or epitope on a target antigen. A multi-specific antibody can be made, for example, by combining heavy chains and/or light chains that recognize different epitopes of the same or different antigen. In some embodiments, by molecular function, a multi-specific binding agent binds one antigen (or epitope) on one of its binding arms (one VH/VL pair), and binds a different antigen (or epitope) on a different binding arm (a different VH/VL pair). By this definition, a multi-specific binding agent has multiple distinct antigen binding arms (in both specificity and CDR sequences).

Multi-specific antibodies and multi-specific antibody fragments of the present technology have at least one arm that specifically binds to, for example, TSHR and at least one other arm that specifically binds to a distinct target antigen. In some embodiments, the distinct target antigen is an antigen or epitope of a B-cell, a T-cell, a myeloid cell, a plasma cell, or a mast-cell. Additionally or alternatively, in certain embodiments, the distinct target antigen is one or more of the antigens selected from the group consisting of CD3, CD4, CD8, CD20, CD19, CD21, CD23, CD46, CD80, HLA-DR, CD74, CD22, CD14, CD15, CD16, CD123, TCR gamma/delta, NKp46, KIR, PD-1, PD-L1, CD28, B7H3, and HER2. In certain embodiments, the multspecific antibodies are capable of binding to tumor cells that express TSHR antigen on the cell surface. In some embodiments, the multspecific antibodies have been engineered to facilitate killing of tumor cells by directing (or recruiting) cytotoxic T cells to a tumor site. Other exemplary multspecific antibodies include those with a first antigen binding site specific for TSHR and an additional antigen binding site specific for a small molecule hapten (e.g., DTP A, IMP288, DOTA, DOTA-Bn, DOTA-desferrioxamine, DOTA (metal) complex, benzyl-DOTA (metal) complex, proteus-DOTA (metal) complex, NOGADA-proteus-DOTA (metal) complex, Star-DFO (metal) complex, DFO (metal) complex, other DOTA-chelates described herein, Biotin, fluorescein, or those disclosed in Goodwin, D A. et al, 1994, Cancer Res. 54 (22): 5937-5946). In some embodiments, the multi-specific antibody or multi-specific antigen binding fragment comprises a catalytic antibody, an immune checkpoint inhibitor, or an immune checkpoint activator.

A variety of multi-specific fusion proteins can be produced using molecular engineering. For example, multi-specific antibodies have been constructed that either utilize the full immunoglobulin framework (e.g., IgG), single chain variable fragment (scFv), or combinations thereof. In some embodiments, the multi-specific fusion protein is divalent, comprising, for example, a scFv with a single binding site for one antigen and a Fab fragment with a single binding site for a second antigen. In some embodiments, the multi-specific fusion protein is divalent, comprising, for example, a scFv with a single binding site for one antigen and another scFv fragment with a single binding site for a second antigen. In other embodiments, the multi-specific fusion protein is tetravalent, comprising, for example, an immunoglobulin (e.g., IgG) with two binding sites for one antigen and two identical scFvs for a second antigen. In other embodiments, the multi-specific fusion protein is tetravalent, comprising, for example, an immunoglobulin (e.g., IgG) with two binding sites for one antigen, one scFv for a second antigen, and one scFv for a third antigen. In other embodiments, the multi-specific fusion protein is tetravalent, comprising, for example, an immunoglobulin (e.g., IgG) with one binding site for one antigen and one binding site for second antigen, one scFv for a third antigen, and one scFv for a fourth antigen.

BsAbs composed of two scFv units in tandem have been shown to be a clinically successful bispecific antibody format. In some embodiments, multi-specific antibodies comprise two single chain variable fragments (scFvs) in tandem have been designed such that a scFv that binds a tumor antigen (e.g., TSHR) is linked with a scFv that engages T cells (e.g., by binding CD3). In this way, T cells are recruited to a tumor site such that they can mediate cytotoxic killing of the tumor cells. See e.g., Dreier et al., J. Immunol. 170:4397-4402 (2003); Bargou et al., Science 321:974-977 (2008).

Recent methods for producing BsAbs include engineered recombinant monoclonal antibodies which have additional cysteine residues so that they crosslink more strongly than the more common immunoglobulin isotypes. See, e.g., FitzGerald et al., Protein Eng. 10 (10): 1221-1225 (1997). Another approach is to engineer recombinant fusion proteins linking two or more different single-chain antibody or antibody fragment segments with the needed dual specificities. See, e.g., Coloma et al., Nature Biotech. 15:159-163 (1997). A variety of bispecific fusion proteins can be produced using molecular engineering.

Bispecific fusion proteins linking two or more different single-chain antibodies or antibody fragments are produced in a similar manner. Recombinant methods can be used to produce a variety of fusion proteins. In some certain embodiments, a BsAb according to the present technology comprises an immunoglobulin, which immunoglobulin comprises a heavy chain and a light chain, and an scFv. In some certain embodiments, the scFv is linked to the C-terminal end of the heavy chain of any TSHR immunoglobulin disclosed herein (e.g., IgG(H)-scFv). In some certain embodiments, scFvs are linked to the C-terminal end of the light chain of any TSHR immunoglobulin disclosed herein (e.g., IgG(L)-scFv). In some embodiments, administration of the IgG(L)-scFv bispecific antibody inhibits cancer progression and/or proliferation in the subject to a greater degree compared to an anti-TSHR×CD3 monomeric BITE, an anti-TSHR×CD3 dimeric BITE, an anti-TSHR×CD3 BITE-Fc, an anti-TSHR×CD3 IgG heterodimer, or an anti-TSHR×CD3 IgG (H)-scFv.

In various embodiments, scFvs are linked to heavy or light chains via a linker sequence. Appropriate linker sequences necessary for the in-frame connection of the heavy chain Fd to the scFv are introduced into the V_{L} and Vₖₐₚₚₐ domains through PCR reactions. The DNA fragment encoding the scFv is then ligated into a staging vector containing a DNA sequence encoding the CH1 domain. The resulting scFv-CH1 construct is excised and ligated into a vector containing a DNA sequence encoding the V_{H} region of a TSHR multi-specific antibody. The resulting vector can be used to transfect an appropriate host cell, such as a mammalian cell for the expression of the bispecific fusion protein.

In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide (e.g., first and/or second antigen binding sites). In some embodiments, a linker is employed in a multi-specific antibody described herein based on specific properties imparted to the multi-specific antibody such as, for example, an increase in stability. In some embodiments, a multi-specific antibody of the present technology comprises a G₄S linker. In some certain embodiments, a multi-specific antibody of the present technology comprises a (G₄S)ₙ linker, wherein n is 1, 2, 3, 4, 5, 6, 7, 8, 9, 10, 11, 12, 13, 14, 15 or more.

Fc Modifications. In some embodiments, the anti-TSHR multi-specific antibodies of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, an anti-TSHR multi-specific antibody of the present technology has an altered affinity for activating and/or inhibitory receptors, having a variant Fc region with one or more amino acid modifications, wherein said one or more amino acid modification is a N297 substitution with alanine, or a K322 substitution with alanine.

Glycosylation Modifications. In some embodiments, anti-TSHR multi-specific antibodies of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the antibodies of the present technology, may have a modified glycosylation site relative to an appropriate reference antibody that binds to an antigen of interest (e.g., TSHR), without altering the functionality of the antibody, e.g., binding activity to the antigen. As used herein, “glycosylation sites” include any specific amino acid sequence in an antibody to which an oligosaccharide (i.e., carbohydrates containing two or more simple sugars linked together) will specifically and covalently attach.

Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform (hTSHR-IgGln) that lacks certain oligosaccharides including fucose and terminal N-acetylglucosamine may be produced in special CHO cells and exhibit enhanced ADCC effector function.

In some embodiments, the carbohydrate content of an immunoglobulin-related composition disclosed herein is modified by adding or deleting a glycosylation site. Methods for modifying the carbohydrate content of antibodies are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of an antibody (or relevant portion or component thereof) is modified by deleting one or more endogenous carbohydrate moieties of the antibody. In some certain embodiments, the present technology includes deleting the glycosylation site of the Fc region of an antibody, by modifying position 297 from asparagine to alanine.

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17:176-180; Davies et al., 2001, Biotechnol. Bioeng. 74:288-294; Shields et al., 2002, J. Biol. Chem. 277:26733-26740; Shinkawa et al., 2003, J. Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336:1239-49.

Fusion Proteins. In one embodiment, the anti-TSHR multi-specific antibody of the present technology is a fusion protein. The anti-TSHR multi-specific antibodies of the present technology, when fused to a second protein, can be used as an antigenic tag. Examples of domains that can be fused to polypeptides include not only heterologous signal sequences, but also other heterologous functional regions. The fusion does not necessarily need to be direct, but can occur through linker sequences. Moreover, fusion proteins of the present technology can also be engineered to improve characteristics of the anti-TSHR multi-specific antibodies. For instance, a region of additional amino acids, particularly charged amino acids, can be added to the N-terminus of the anti-TSHR multi-specific antibody to improve stability and persistence during purification from the host cell or subsequent handling and storage. Also, peptide moieties can be added to an anti-TSHR multi-specific antibody to facilitate purification. Such regions can be removed prior to final preparation of the anti-TSHR multi-specific antibody. The addition of peptide moieties to facilitate handling of polypeptides are familiar and routine techniques in the art. The anti-TSHR multi-specific antibody of the present technology can be fused to marker sequences, such as a peptide which facilitates purification of the fused polypeptide. In select embodiments, the marker amino acid sequence is a hexa-histidine peptide, such as the tag provided in a pQE vector (QIAGEN, Inc., Chatsworth, Calif), among others, many of which are commercially available. As described in Gentz et al., Proc. Natl. Acad. Sci. USA 86:821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37:767, 1984.

Thus, any of these above fusion proteins can be engineered using the polynucleotides or the polypeptides of the present technology. Also, in some embodiments, the fusion proteins described herein show an increased half-life in vivo.

Fusion proteins having disulfide-linked dimeric structures (due to the IgG) can be more efficient in binding and neutralizing other molecules compared to the monomeric secreted protein or protein fragment alone. Fountoulakis et al., J. Biochem. 270:3958-3964, 1995.

Similarly, EP-A-O 464 533 (Canadian counterpart 2045869) discloses fusion proteins comprising various portions of constant region of immunoglobulin molecules together with another human protein or a fragment thereof. In many cases, the Fc part in a fusion protein is beneficial in therapy and diagnosis, and thus can result in, e.g., improved pharmacokinetic properties. See EP-A 0232 262. Alternatively, deleting or modifying the Fc part after the fusion protein has been expressed, detected, and purified, may be desired. For example, the Fc portion can hinder therapy and diagnosis if the fusion protein is used as an antigen for immunizations. In drug discovery, e.g., human proteins, such as hIL-5, have been fused with Fc portions for the purpose of high-throughput screening assays to identify antagonists of hIL-5. Bennett et al., J. Molecular Recognition 8:52-58, 1995; Johanson et al., J. Biol. Chem., 270:9459-9471, 1995.

Labeled Anti-TSHR multi-specific antibodies. In one embodiment, the anti-TSHR multi-specific antibody of the present technology is coupled with a label moiety, i.e., detectable group. The particular label or detectable group conjugated to the anti-TSHR multi-specific antibody is not a critical aspect of the technology, so long as it does not significantly interfere with the specific binding of the anti-TSHR multi-specific antibody of the present technology to the TSHR protein. The detectable group can be any material having a detectable physical or chemical property. Such detectable labels have been well-developed in the field of immunoassays and imaging. In general, almost any label useful in such methods can be applied to the present technology. Thus, a label is any composition detectable by spectroscopic, photochemical, biochemical, immunochemical, electrical, optical or chemical means. Labels useful in the practice of the present technology include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹³¹I, ¹¹²In, ^{99m}Tc), other imaging agents such as microbubbles (for ultrasound imaging), ¹⁸F, ¹¹C, ¹⁵O, ⁸⁹Zr (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that describe the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene OR).

The label can be coupled directly or indirectly to the desired component of an assay according to methods well known in the art. As indicated above, a wide variety of labels can be used, with the choice of label depending on factors such as required sensitivity, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, e.g., biotin, thyroxine, and cortisol, it can be used in conjunction with the labeled, naturally-occurring anti-ligands. Alternatively, any haptenic or antigenic compound can be used in combination with an antibody, e.g., an anti-TSHR multi-specific antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labeling moieties, include, but are not limited to, e.g., fluorescein and its derivatives, rhodamine and its derivatives, dansyl, umbelliferone, and the like. Chemiluminescent compounds useful as labeling moieties, include, but are not limited to, e.g., luciferin, and 2,3-dihydrophthalazinediones, e.g., luminol. For a review of various labeling or signal-producing systems which can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally, simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies, e.g., the anti-TSHR multi-specific antibodies. In this case, antigen-coated particles are agglutinated by samples comprising the target antibodies. In this format, none of the components need be labeled and the presence of the target antibody is detected by simple visual inspection.

### B. Identifying and Characterizing the Anti-TSHR Multi-Specific Antibodies of the Present Technology

Methods for identifying and or screening the anti-TSHR multi-specific antibodies of the present technology. Methods useful to identify and screen antibodies against TSHR polypeptides for those that possess the desired specificity to TSHR protein (e.g., those that bind to the extracellular domain of TSHR protein (e.g., comprising the amino acids at positions 22-260 of SEQ ID NO: 74) include any immunologically-mediated techniques known within the art. Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity; (2) helper T lymphocytes can be incubated with antigens and antigen presenting cells and the synthesis and secretion of cytokines measured by standard methods (Windhagen A et al., Immunity, 2:373-80, 1995); (3) antigen presenting cells can be incubated with whole protein antigen and the presentation of that antigen on MHC detected by either T lymphocyte activation assays or biophysical methods (Harding et al., Proc. Natl. Acad. Sci., 86:4230-4, 1989); (4) mast cells can be incubated with reagents that cross-link their Fc-epsilon receptors and histamine release measured by enzyme immunoassay (Siraganian et al., TIPS, 4:432-437, 1983); and (5) enzyme-linked immunosorbent assay (ELISA).

Similarly, products of an immune response in either a model organism (e.g., mouse) or a human subject can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood, 72:1310-5, 1988); (3) the proliferation of peripheral blood mononuclear cells (PBMCs) in response to mitogens or mixed lymphocyte reaction can be measured using 3H-thymidine; (4) the phagocytic capacity of granulocytes, macrophages, and other phagocytes in PBMCs can be measured by placing PBMCs in wells together with labeled particles (Peters et al., Blood, 72:1310-5, 1988); and (5) the differentiation of immune system cells can be measured by labeling PBMCs with antibodies to CD molecules such as CD4 and CD8 and measuring the fraction of the PBMCs expressing these markers.

In one embodiment, anti-TSHR multi-specific antibodies of the present technology are selected using display of TSHR peptides on the surface of replicable genetic packages. See, e.g., U.S. Pat. Nos. 5,514,548; 5,837,500; 5,871,907; 5,885,793; 5,969,108; 6,225,447; 6,291,650; 6,492,160; EP 585 287; EP 605522; EP 616640; EP 1024191; EP 589 877; EP 774 511; EP 844 306. Methods useful for producing/selecting a filamentous bacteriophage particle containing a phagemid genome encoding for a binding molecule with a desired specificity has been described. See, e.g., EP 774 511; U.S. Pat. Nos. 5,871,907; 5,969,108; 6,225,447; 6,291,650; 6,492,160.

In some embodiments, anti-TSHR multi-specific antibodies of the present technology are selected using display of TSHR peptides on the surface of a yeast host cell. Methods useful for the isolation of scFv polypeptides by yeast surface display have been described by Kieke et al., Protein Eng. 1997 November; 10 (11): 1303-10.

In some embodiments, anti-TSHR multi-specific antibodies of the present technology are selected using ribosome display. Methods useful for identifying ligands in peptide libraries using ribosome display have been described by Mattheakis et al., Proc. Natl. Acad. Sci. USA 91:9022-26, 1994; and Hanes et al., Proc. Natl. Acad. Sci. USA 94:4937-42, 1997.

In certain embodiments, anti-TSHR multi-specific antibodies of the present technology are selected using tRNA display of TSHR peptides. Methods useful for in vitro selection of ligands using tRNA display have been described by Merryman et al., Chem. Biol., 9:741-46, 2002.

In one embodiment, anti-TSHR multi-specific antibodies of the present technology are selected using RNA display. Methods useful for selecting peptides and proteins using RNA display libraries have been described by Roberts et al. Proc. Natl. Acad. Sci. USA, 94:12297-302, 1997; and Nemoto et al., FEBS Lett., 414:405-8, 1997. Methods useful for selecting peptides and proteins using unnatural RNA display libraries have been described by Frankel et al., Curr. Opin. Struct. Biol., 13:506-12, 2003.

In some embodiments, anti-TSHR multi-specific antibodies of the present technology are expressed in the periplasm of gram negative bacteria and mixed with labeled TSHR protein. See WO 02/34886. In clones expressing recombinant polypeptides with affinity for TSHR protein, the concentration of the labeled TSHR protein bound to the anti-TSHR multi-specific antibodies is increased and allows the cells to be isolated from the rest of the library as described in Harvey et al., Proc. Natl. Acad. Sci. 22:9193-98 2004 and U.S. Pat. Publication No. 2004/0058403.

After selection of the desired anti-TSHR multi-specific antibodies, it is contemplated that said antibodies can be produced in large volume by any technique known to those skilled in the art, e.g., prokaryotic or eukaryotic cell expression and the like. The anti-TSHR multi-specific antibodies which are, e.g., but not limited to, anti-TSHR hybrid antibodies or fragments can be produced by using conventional techniques to construct an expression vector that encodes an antibody heavy chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which can be manipulated as described herein, thereby producing a vector for the expression of a hybrid antibody heavy chain.

Measurement of TSHR Binding. In some embodiments, a TSHR binding assay refers to an assay format wherein TSHR protein and an anti-TSHR multi-specific antibody are mixed under conditions suitable for binding between the TSHR protein and the anti-TSHR multi-specific antibody and assessing the amount of binding between the TSHR protein and the anti-TSHR multi-specific antibody. The amount of binding is compared with a suitable control, which can be the amount of binding in the absence of the TSHR protein, the amount of the binding in the presence of a non-specific immunoglobulin composition, or both. The amount of binding can be assessed by any suitable method. Binding assay methods include, e.g., ELISA, radioimmunoassays, scintillation proximity assays, fluorescence energy transfer assays, liquid chromatography, membrane filtration assays, and the like. Biophysical assays for the direct measurement of TSHR protein binding to anti-TSHR multi-specific antibody are, e.g., nuclear magnetic resonance, fluorescence, fluorescence polarization, surface plasmon resonance (BIACORE chips) and the like. Specific binding is determined by standard assays known in the art, e.g., radioligand binding assays, ELISA, FRET, immunoprecipitation, SPR, NMR (2D-NMR), mass spectroscopy and the like. If the specific binding of a candidate anti-TSHR multi-specific antibody is at least 1 percent greater than the binding observed in the absence of the candidate anti-TSHR multi-specific antibody, the candidate anti-TSHR multi-specific antibody is useful as an anti-TSHR multi-specific antibody of the present technology.

### Uses of the Anti-TSHR Multi-Specific Antibodies of the Present Technology

General. The anti-TSHR multi-specific antibodies of the present technology are useful in methods known in the art relating to the localization and/or quantitation of TSHR protein (e.g., for use in measuring levels of the TSHR protein within appropriate physiological samples, for use in diagnostic methods, for use in imaging the polypeptide, and the like). Antibodies of the present technology are useful to isolate a TSHR protein by standard techniques, such as affinity chromatography or immunoprecipitation. An anti-TSHR multi-specific antibody of the present technology can facilitate the purification of natural immunoreactive TSHR proteins from biological samples, e.g., mammalian sera or cells as well as recombinantly-produced immunoreactive TSHR proteins expressed in a host system. Moreover, anti-TSHR multi-specific antibodies of the present technology can be used to detect an immunoreactive TSHR protein (e.g., in plasma, a cellular lysate or cell supernatant) in order to evaluate the abundance and pattern of expression of the immunoreactive polypeptide. The anti-TSHR multi-specific antibodies of the present technology can be used diagnostically to monitor immunoreactive TSHR protein levels in tissue as part of a clinical testing procedure, e.g., to determine the efficacy of a given treatment regimen. As noted above, the detection can be facilitated by coupling (i.e., physically linking) the anti-TSHR multi-specific antibodies of the present technology to a detectable substance.

Detection of TSHR protein. An exemplary method for detecting the presence or absence of an immunoreactive TSHR protein in a biological sample involves obtaining a biological sample from a test subject and contacting the biological sample with an anti-TSHR multi-specific antibody of the present technology capable of detecting an immunoreactive TSHR protein such that the presence of an immunoreactive TSHR protein is detected in the biological sample. Detection may be accomplished by means of a detectable label attached to the antibody.

The term “labeled” with regard to the anti-TSHR multi-specific antibody is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In some embodiments, the anti-TSHR multi-specific antibodies disclosed herein are conjugated to one or more detectable labels. For such uses, anti-TSHR multi-specific antibodies may be detectably labeled by covalent or non-covalent attachment of a chromogenic, enzymatic, radioisotopic, isotopic, fluorescent, toxic, chemiluminescent, nuclear magnetic resonance contrast agent or other label. Examples of suitable chromogenic labels include diaminobenzidine and 4-hydroxyazo-benzene-2-carboxylic acid. Examples of suitable enzyme labels include malate dehydrogenase, staphylococcal nuclease, Δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is an exemplary isotope where in vivo imaging is used since it avoids the problem of dehalogenation of the ¹²⁵I or ¹³¹I-labeled TSHR-binding antibodies by the liver. In addition, this isotope has a more favorable gamma emission energy for imaging (Perkins et al, Eur. J. Nucl. Med. 70:296-301 (1985); Carasquillo et al., J. Nucl. Med. 25:281-287 (1987)). For example, ¹¹¹In coupled to monoclonal antibodies with 1-(P-isothiocyanatobenzyl)-DPTA exhibits little uptake in non-tumorous tissues, particularly the liver, and enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The detection method of the present technology can be used to detect an immunoreactive TSHR protein in a biological sample in vitro as well as in vivo. In vitro techniques for detection of an immunoreactive TSHR protein include enzyme linked immunosorbent assays (ELISAs), Western blots, immunoprecipitations, radioimmunoassay, and immunofluorescence. Furthermore, in vivo techniques for detection of an immunoreactive TSHR protein include introducing into a subject a labeled anti-TSHR multi-specific antibody of the present technology. For example, the anti-TSHR multi-specific antibody can be labeled with a radioactive marker whose presence and location in a subject can be detected by standard imaging techniques. In one embodiment, the biological sample contains TSHR protein molecules from the test subject.

Immunoassay and Imaging. An anti-TSHR multi-specific antibody of the present technology can be used to assay immunoreactive TSHR protein levels in a biological sample (e.g., human plasma) using antibody-based techniques. For example, protein expression in tissues can be studied with classical immunohistological methods. Jalkanen, M. et al., J Cell Biol 101:976-985, 1985; Jalkanen, M. et al., J Cell Biol 105:3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein, rhodamine, and green fluorescent protein (GFP), as well as biotin.

In addition to assaying immunoreactive TSHR protein levels in a biological sample, anti-TSHR multi-specific antibodies of the present technology may be used for in vivo imaging of TSHR. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-TSHR multi-specific antibodies by labeling of nutrients for the relevant scFv clone.

An anti-TSHR multi-specific antibody of the present technology which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled anti-TSHR multi-specific antibody will then accumulate at the location of cells which contain the specific target polypeptide. For example, labeled anti-TSHR multi-specific antibodies of the present technology will accumulate within the subject in cells and tissues in which the TSHR protein has localized.

Thus, the present technology provides a diagnostic method of a medical condition, which involves: (a) assaying the expression of immunoreactive TSHR protein by measuring binding of an anti-TSHR multi-specific antibody of the present technology in cells or body fluid of an individual; (b) comparing the amount of immunoreactive TSHR protein present in the sample with a standard reference, wherein an increase or decrease in immunoreactive TSHR protein levels compared to the standard is indicative of a medical condition.

Affinity Purification. The anti-TSHR multi-specific antibodies of the present technology may be used to purify immunoreactive TSHR protein from a sample. In some embodiments, the antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating or rocking the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl) propionic acid can be employed as a means for cleaving a polypeptide from a solid support. (Brown et al., Mol. Divers, pp, 4-12 (1995); Rothschild et al., Nucl Acids Res, 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.)

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

### A. Diagnostic Uses of Anti-TSHR Multi-Specific Antibodies of the Present Technology

General. The anti-TSHR multi-specific antibodies of the present technology are useful in diagnostic methods. As such, the present technology provides methods using the antibodies in the diagnosis of TSHR activity in a subject. Anti-TSHR multi-specific antibodies of the present technology may be selected such that they have any level of epitope binding specificity and very high binding affinity to a TSHR protein. In general, the higher the binding affinity of an antibody the more stringent wash conditions can be performed in an immunoassay to remove nonspecifically bound material without removing target polypeptide. Accordingly, anti-TSHR multi-specific antibodies of the present technology useful in diagnostic assays usually have binding affinities of about 10⁸ M⁻¹, 10⁹ M⁻¹, 10¹⁰ M⁻¹, 10¹¹ M⁻¹ or 10¹² M⁻¹ to TSHR. Further, it is desirable that anti-TSHR multi-specific antibodies used as diagnostic reagents have a sufficient kinetic on-rate to reach equilibrium under standard conditions in at least 12 h, at least five (5) h, or at least one (1) hour.

Anti-TSHR multi-specific antibodies can be used to detect an immunoreactive TSHR protein in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject. In certain embodiments, the subject is at an early stage of cancer. In one embodiment, the early stage of cancer is determined by the level or expression pattern of TSHR protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. See U.S. Pat. Nos. 4,376,110, 4,486,530, 5,914,241, and 5,965,375. Such assays use one antibody, e.g., an anti-TSHR multi-specific antibody or a population of anti-TSHR multi-specific antibodies, e.g., the anti-TSHR multi-specific antibodies of the present technology, immobilized to a solid phase, and another anti-TSHR multi-specific antibody or a population of anti-TSHR multi-specific antibodies in solution. Typically, the solution anti-TSHR multi-specific antibody or population of anti-TSHR multi-specific antibodies is labeled. If an antibody population is used, the population can contain antibodies binding to different epitope specificities within the target polypeptide. Accordingly, the same population can be used for both solid phase and solution antibody. If anti-TSHR monoclonal multi-specific antibodies are used, first and second anti-TSHR monoclonal multi-specific antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the TSHR protein with the anti-TSHR multi-specific antibody, a sample is incubated for a period that usually varies from about 10 min to about 24 hr and is usually about 1 hr. A wash step is then performed to remove components of the sample not specifically bound to the anti-TSHR multi-specific antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive TSHR protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the TSHR protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-TSHR multi-specific antibodies can be joined to a linker molecule, such as biotin for attachment to a surface bound linker, such as avidin.

In some embodiments, the present disclosure provides an anti-TSHR multi-specific antibody of the present technology conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. In some embodiments, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates may be coupled to the antibodies of the present technology using standard chemistries. The chelate is normally linked to the antibody by a group which enables formation of a bond to the molecule with minimal loss of immunoreactivity and minimal aggregation and/or internal cross-linking. Other methods and reagents for conjugating chelates to antibodies are disclosed in U.S. Pat. No. 4,824,659. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MRI, when used along with the TSHR multi-specific antibodies of the present technology.

Macrocyclic chelates such as NOTA (1,4,7-triaza-cyclononane-N,N′,N″-triacetic acid), DOTA, and TETA (p-bromoacetamido-benzyl-tetraethylaminetetraacetic acid) are of use with a variety of metals and radiometals, such as radionuclides of gallium, yttrium and copper, respectively. Such metal-chelate complexes can be stabilized by tailoring the ring size to the metal of interest. Examples of other DOTA chelates include (i) DOTA-Phe-Lys(HSG)-D-Tyr-Lys(HSG)-NH₂; (ii) Ac-Lys(HSG)D-Tyr-Lys(HSG)-Lys(Tscg-Cys)-NH₂; (iii) DOTA-D-Asp-D-Lys(HSG)-D-Asp-D-Lys(HSG)-NH₂; (iv) DOTA-D-Glu-D-Lys(HSG)-D-Glu-D-Lys(HSG)-NH₂; (v) DOTA-D-Tyr-D-Lys(HSG)-D-Glu-D-Lys(HSG)-NH₂; (vi) DOTA-D-Ala-D-Lys(HSG)-D-Glu-D-Lys(HSG)-NH₂; (vii) DOTA-D-Phe-D-Lys(HSG)-D-Tyr-D-Lys(HSG)-NH₂; (viii) Ac-D-Phe-D-Lys(DOTA)-D-Tyr-D-Lys(DOTA)-NH₂; (ix) Ac-D-Phe-D-Lys(DTPA)-D-Tyr-D-Lys(DTPA)-NH₂; (x) Ac-D-Phe-D-Lys(Bz-DTPA)-D-Tyr-D-Lys(Bz-DTPA)-NH₂; (xi) Ac-D-Lys(HSG)-D-Tyr-D-Lys(HSG)-D-Lys(Tscg-Cys)-NH₂; (xii) DOTA-D-Phe-D-Lys(HSG)-D-Tyr-D-Lys(HSG)-D-Lys(Tscg-Cys)-NH₂; (xiii) (Tscg-Cys)-D-Phe-D-Lys(HSG)-D-Tyr-D-Lys(HSG)-D-Lys(DOTA)-NH₂; (xiv) Tscg-D-Cys-D-Glu-D-Lys(HSG)-D-Glu-D-Lys(HSG)-NH₂; (xv) (Tscg-Cys)-D-Glu-D-Lys(HSG)-D-Glu-D-Lys(HSG)-NH₂; (xvi) Ac-D-Cys-D-Lys(DOTA)-D-Tyr-D-Ala-D-Lys(DOTA)-D-Cys-NH₂; (xvii) Ac-D-Cys-D-Lys(DTPA)-D-Tyr-D-Lys(DTPA)-NH₂; (xviii) Ac-D-Lys(DTPA)-D-Tyr-D-Lys(DTPA)-D-Lys(Tscg-Cys)-NH₂; and (xix) Ac-D-Lys(DOTA)-D-Tyr-D-Lys(DOTA)-D-Lys(Tscg-Cys)-NH₂.

Other ring-type chelates such as macrocyclic polyethers, which are of interest for stably binding nuclides, such as ²²³Ra for RAIT are also contemplated.

### B. Therapeutic Use of Anti-TSHR Multi-Specific Antibodies of the Present Technology

In one aspect, the immunoglobulin-related compositions (e.g., multi-specific antibodies or antigen binding fragments thereof) of the present technology are useful for the treatment of TSHR-associated pathologies, such as thyroid cancers, T-ALL (T lineage acute lymphoblastic leukemia), multiple myeloma, lung cancers, colorectal cancers, gastric cancers, liver cancers, pancreatic cancers, urothelial cancers, breast cancers, ovarian cancers, Graves' disease, thyroid-associated ophthalmopathy (TAO), osteoporosis, and adipogenesis. In some embodiments, the TSHR-associated pathology is a solid tumor or liquid tumor. Such treatment can be used in patients identified as having pathologically high levels of the TSHR (e.g., those diagnosed by the methods described herein) or in patients diagnosed with a disease known to be associated with such pathological levels.

In one aspect, the present disclosure provides a method for treating a TSHR-associated pathology in a subject in need thereof, comprising administering to the subject an effective amount of an antibody (or antigen binding fragment thereof) of the present technology. Examples of TSHR-associated pathologies that can be treated by the immunoglobulin-related compositions of the present technology include, but are not limited to: cancer, Graves' disease, thyroid-associated ophthalmopathy (TAO), osteoporosis, and adipogenesis. In some embodiments, the anti-TSHR multi-specific antibodies of the present technology are useful for modulating weight gain (either to increase body weight or to decrease body weight). In other embodiments, the anti-TSHR multi-specific antibodies of the present technology are useful for decreasing bone remodeling to treat osteoporosis. Additionally or alternatively, in some embodiments, the methods of the present technology further comprise administering to the subject an effective amount of a multi-specific antibody that specifically binds to HER2 and T cells. In certain embodiments, the multi-specific antibody that specifically binds to HER2 and T cells comprises a light chain amino acid sequence of SEQ ID NO: 54 and a heavy chain amino acid sequence of SEQ ID NO: 56. Examples of TSHR-positive cancer include thyroid cancers, T-ALL (T lineage acute lymphoblastic leukemia), multiple myeloma, lung cancers, colorectal cancers, gastric cancers, liver cancers, pancreatic cancers, urothelial cancers, breast cancers, ovarian cancers and the like. In certain embodiments, the TSHR-positive cancer is resistant to a RET inhibitor, a NTRK inhibitor, an ALK inhibitor, a RAF inhibitor, or a MEK kinase inhibitor.

The compositions of the present technology may be employed in conjunction with other therapeutic agents useful in the treatment of TSHR-associated pathologies, such as TSHR(+) cancers, Graves' disease, thyroid-associated ophthalmopathy (TAO), osteoporosis, and adipogenesis. For example, the antibodies or antigen binding fragments of the present technology may be separately, sequentially or simultaneously administered with at least one additional therapeutic agent.

Examples of additional therapeutic agents include, but are not limited to, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Additionally or alternatively, in some embodiments, the TSHR multi-specific antibodies or antigen binding fragments of the present technology may be separately, sequentially or simultaneously administered with at least one additional immuno-modulating/stimulating antibody including but not limited to anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

The compositions of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In some embodiments, the antibodies of the present technology comprise pharmaceutical formulations which may be administered to subjects in need thereof in one or more doses. Dosage regimens can be adjusted to provide the desired response (e.g., a therapeutic response).

Typically, an effective amount of the antibody compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-TSHR multi-specific antibodies, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of antibody ranges from 0.1-10,000 micrograms per kg body weight. In one embodiment, antibody concentrations in a carrier range from 0.2 to 2000 micrograms per delivered milliliter. An exemplary treatment regime entails administration once per every two weeks or once a month or once every 3 to 6 months. Anti-TSHR multi-specific antibodies may be administered on multiple occasions. Intervals between single dosages can be hourly, daily, weekly, monthly or yearly. Intervals can also be irregular as indicated by measuring blood levels of the antibody in the subject. In some methods, dosage is adjusted to achieve a serum antibody concentration in the subject of from about 75 μg/mL to about 125 μg/mL, 100 μg/mL to about 150 μg/mL, from about 125 μg/mL to about 175 μg/mL, or from about 150 μg/mL to about 200 μg/mL. Alternatively, anti-TSHR multi-specific antibodies can be administered as a sustained release formulation, in which case less frequent administration is required. Dosage and frequency vary depending on the half-life of the antibody in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In another aspect, the present disclosure provides a method for detecting cancer in a subject in vivo comprising (a) administering to the subject an effective amount of an antibody (or antigen binding fragment thereof) of the present technology, wherein the antibody is configured to localize to a cancer cell expressing TSHR and is labeled with a radioisotope; and (b) detecting the presence of a tumor in the subject by detecting radioactive levels emitted by the antibody that are higher than a reference value. In some embodiments, the reference value is expressed as injected dose per gram (% ID/g). The reference value may be calculated by measuring the radioactive levels present in non-tumor (normal) tissues, and computing the average radioactive levels present in non-tumor (normal) tissues±standard deviation. In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In some embodiments, the subject is diagnosed with or is suspected of having cancer. Radioactive levels emitted by the antibody may be detected using positron emission tomography or single photon emission computed tomography.

Additionally or alternatively, in some embodiments, the method further comprises administering to the subject an effective amount of an immunoconjugate comprising an antibody of the present technology conjugated to a radionuclide. In some embodiments, the radionuclide is an alpha particle-emitting isotope, a beta particle-emitting isotope, an Auger-emitter, or any combination thereof. Examples of beta particle-emitting isotopes include ⁸⁶Y, ⁹⁰Y, ⁸⁹Sr, ¹⁶⁵Dy, ¹⁸⁶Re, ¹⁸⁸Re, ¹⁷⁷Lu, and ⁶⁷Cu. Examples of alpha particle-emitting isotopes include ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, and ²⁵⁵Fm. Examples of Auger-emitters include ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, and ²⁰³Pb. In some embodiments of the method, nonspecific FcR-dependent binding in normal tissues is eliminated or reduced (e.g., via N297A mutation in Fc region, which results in aglycosylation). The therapeutic effectiveness of such an immunoconjugate may be determined by computing the area under the curve (AUC) tumor: AUC normal tissue ratio. In some embodiments, the immunoconjugate has a AUC tumor: AUC normal tissue ratio of about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

PRIT. In one aspect, the present disclosure provides a method for detecting tumors in a subject in need thereof comprising (a) administering to the subject an effective amount of a complex comprising a radiolabeled DOTA hapten and a multi-specific antibody of the present technology that binds to the radiolabeled DOTA hapten, a CD3 antigen and a TSHR antigen, wherein the complex is configured to localize to a tumor expressing the TSHR antigen recognized by the multi-specific antibody of the complex; and (b) detecting the presence of solid tumors in the subject by detecting radioactive levels emitted by the complex that are higher than a reference value. In some embodiments, the subject is human.

In another aspect, the present disclosure provides a method for selecting a subject for pretargeted radioimmunotherapy comprising (a) administering to the subject an effective amount of a complex comprising a radiolabeled DOTA hapten and a multi-specific antibody of the present technology that binds to the radiolabeled DOTA hapten, a CD3 antigen and a TSHR antigen, wherein the complex is configured to localize to a tumor expressing the TSHR antigen recognized by the multi-specific antibody of the complex; (b) detecting radioactive levels emitted by the complex; and (c) selecting the subject for pretargeted radioimmunotherapy when the radioactive levels emitted by the complex are higher than a reference value. In some embodiments, the subject is human.

Examples of DOTA haptens include (i) DOTA-Phe-Lys(HSG)-D-Tyr-Lys(HSG)-NH₂; (ii) Ac-Lys(HSG)D-Tyr-Lys(HSG)-Lys(Tscg-Cys)-NH₂; (iii) DOTA-D-Asp-D-Lys(HSG)-D-Asp-D-Lys(HSG)-NH₂; (iv) DOTA-D-Glu-D-Lys(HSG)-D-Glu-D-Lys(HSG)-NH₂; (v) DOTA-D-Tyr-D-Lys(HSG)-D-Glu-D-Lys(HSG)-NH₂; (vi) DOTA-D-Ala-D-Lys(HSG)-D-Glu-D-Lys(HSG)-NH₂; (vii) DOTA-D-Phe-D-Lys(HSG)-D-Tyr-D-Lys(HSG)-NH₂; (viii) Ac-D-Phe-D-Lys(DOTA)-D-Tyr-D-Lys(DOTA)-NH₂; (ix) Ac-D-Phe-D-Lys(DTPA)-D-Tyr-D-Lys(DTPA)-NH₂; (x) Ac-D-Phe-D-Lys(Bz-DTPA)-D-Tyr-D-Lys(Bz-DTPA)-NH₂; (xi) Ac-D-Lys(HSG)-D-Tyr-D-Lys(HSG)-D-Lys(Tscg-Cys)-NH₂; (xii) DOTA-D-Phe-D-Lys(HSG)-D-Tyr-D-Lys(HSG)-D-Lys(Tscg-Cys)-NH₂; (xiii) (Tscg-Cys)-D-Phe-D-Lys(HSG)-D-Tyr-D-Lys(HSG)-D-Lys(DOTA)-NH₂; (xiv) Tscg-D-Cys-D-Glu-D-Lys(HSG)-D-Glu-D-Lys(HSG)-NH₂; (xv) (Tscg-Cys)-D-Glu-D-Lys(HSG)-D-Glu-D-Lys(HSG)-NH₂; (xvi) Ac-D-Cys-D-Lys(DOTA)-D-Tyr-D-Ala-D-Lys(DOTA)-D-Cys-NH₂; (xvii) Ac-D-Cys-D-Lys(DTPA)-D-Tyr-D-Lys(DTPA)-NH₂; (xviii) Ac-D-Lys(DTPA)-D-Tyr-D-Lys(DTPA)-D-Lys(Tscg-Cys)-NH₂; (xix) Ac-D-Lys(DOTA)-D-Tyr-D-Lys(DOTA)-D-Lys(Tscg-Cys)-NH₂ and (xx) DOTA. The radiolabel may be an alpha particle-emitting isotope, a beta particle-emitting isotope, or an Auger-emitter. Examples of radiolabels include ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu.

In some embodiments of the methods disclosed herein, the radioactive levels emitted by the complex are detected using positron emission tomography or single photon emission computed tomography. Additionally or alternatively, in some embodiments of the methods disclosed herein, the subject is diagnosed with, or is suspected of having a TSHR-associated cancer such as thyroid cancers, T-ALL (T lineage acute lymphoblastic leukemia), multiple myeloma, lung cancers, colorectal cancers, gastric cancers, liver cancers, pancreatic cancers, urothelial cancers, breast cancers, and ovarian cancers.

Additionally or alternatively, in some embodiments of the methods disclosed herein, the complex is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the complex is administered into the cerebral spinal fluid or blood of the subject.

In some embodiments of the methods disclosed herein, the radioactive levels emitted by the complex are detected between 2 to 120 hours after the complex is administered. In certain embodiments of the methods disclosed herein, the radioactive levels emitted by the complex are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45 (9): 1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In another aspect, the present disclosure provides a method for increasing tumor sensitivity to radiation therapy in a subject diagnosed with a TSHR-associated cancer comprising (a) administering an effective amount of an anti-DOTA multi-specific antibody of the present technology to the subject, wherein the anti-DOTA multi-specific antibody is (i) configured to localize to a tumor expressing a TSHR target antigen and (ii) configured to bind a CD3 target antigen on T cells; and (b) administering an effective amount of a radiolabeled-DOTA hapten to the subject, wherein the radiolabeled-DOTA hapten is configured to bind to the anti-DOTA multi-specific antibody. In some embodiments, the subject is human.

The anti-DOTA multi-specific antibody is administered under conditions and for a period of time (e.g., according to a dosing regimen) sufficient for it to saturate tumor cells. In some embodiments, unbound anti-DOTA multi-specific antibody is removed from the blood stream after administration of the anti-DOTA multi-specific antibody. In some embodiments, the radiolabeled-DOTA hapten is administered after a time period that may be sufficient to permit clearance of unbound anti-DOTA multi-specific antibody.

The radiolabeled-DOTA hapten may be administered at any time between 1 minute to 4 or more days following administration of the anti-DOTA multi-specific antibody. For example, in some embodiments, the radiolabeled-DOTA hapten is administered 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, or any range therein, following administration of the anti-DOTA multi-specific antibody. Alternatively, the radiolabeled-DOTA hapten may be administered at any time after 4 or more days following administration of the anti-DOTA multi-specific antibody.

Additionally or alternatively, in some embodiments, the method further comprises administering an effective amount of a clearing agent to the subject prior to administration of the radiolabeled-DOTA hapten. A clearing agent can be any molecule (dextran or dendrimer or polymer) that can be conjugated with C825-hapten. In some embodiments, the clearing agent is no more than 2000 kD, 1500 kD, 1000 kD, 900 kD, 800 kD, 700 kD, 600 kD, 500 kD, 400 kD, 300 kD, 200 kD, 100 kD, 90 kD, 80 kD, 70 kD, 60 kD, 50 kD, 40 kD, 30 kD, 20 kD, 10 kD, or 5 kD. In some embodiments, the clearing agent is a 500 kD aminodextran-DOTA conjugate (e.g., 500 kD dextran-DOTA-Bn (Y), 500 kD dextran-DOTA-Bn (Lu), or 500 kD dextran-DOTA-Bn (In) etc.).

In some embodiments, the clearing agent and the radiolabeled-DOTA hapten are administered without further administration of the anti-DOTA multi-specific antibody of the present technology. For example, in some embodiments, an anti-DOTA multi-specific antibody of the present technology is administered according to a regimen that includes at least one cycle of: (i) administration of the anti-DOTA multi-specific antibody of the present technology (optionally so that relevant tumor cells are saturated); (ii) administration of a radiolabeled-DOTA hapten and, optionally a clearing agent; (iii) optional additional administration of the radiolabeled-DOTA hapten and/or the clearing agent, without additional administration of the anti-DOTA multi-specific antibody. In some embodiments, the method may comprise multiple such cycles (e.g., 1, 2, 3, 4, 5, 6, 7, 8, 9, 10 or more cycles).

Additionally or alternatively, in some embodiments of the method, the anti-DOTA multi-specific antibody and/or the radiolabeled-DOTA hapten is administered intravenously, intramuscularly, intraarterially, intrathecally, intracapsularly, intraorbitally, intradermally, intraperitoneally, transtracheally, subcutaneously, intracerebroventricularly, intratumorally, orally or intranasally.

In one aspect, the present disclosure provides a method for increasing tumor sensitivity to radiation therapy in a subject diagnosed with a TSHR-associated cancer comprising administering to the subject an effective amount of a complex comprising a radiolabeled-DOTA hapten and a multi-specific antibody of the present technology that recognizes and binds to the radiolabeled-DOTA hapten, a CD3 antigen and a TSHR antigen, wherein the complex is configured to localize to a tumor expressing the TSHR antigen recognized by the multi-specific antibody of the complex. The complex may be administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In some embodiments, the subject is human.

In another aspect, the present disclosure provides a method for treating cancer in a subject in need thereof comprising (a) administering an effective amount of an anti-DOTA multi-specific antibody of the present technology to the subject, wherein the anti-DOTA multi-specific antibody is (i) configured to localize to a tumor expressing a TSHR target antigen and (ii) configured to bind a CD3 target antigen on T cells; and (b) administering an effective amount of a radiolabeled-DOTA hapten to the subject, wherein the radiolabeled-DOTA hapten is configured to bind to the anti-DOTA multi-specific antibody. The anti-DOTA multi-specific antibody is administered under conditions and for a period of time (e.g., according to a dosing regimen) sufficient for it to saturate tumor cells. In some embodiments, unbound anti-DOTA multi-specific antibody is removed from the blood stream after administration of the anti-DOTA multi-specific antibody. In some embodiments, the radiolabeled-DOTA hapten is administered after a time period that may be sufficient to permit clearance of unbound anti-DOTA multi-specific antibody. In some embodiments, the subject is human.

Accordingly, in some embodiments, the method further comprises administering an effective amount of a clearing agent to the subject prior to administration of the radiolabeled-DOTA hapten. The radiolabeled-DOTA hapten may be administered at any time between 1 minute to 4 or more days following administration of the anti-DOTA multi-specific antibody. For example, in some embodiments, the radiolabeled-DOTA hapten is administered 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, or any range therein, following administration of the anti-DOTA multi-specific antibody. Alternatively, the radiolabeled-DOTA hapten may be administered at any time after 4 or more days following administration of the anti-DOTA multi-specific antibody.

The clearing agent may be a 500 kD aminodextran-DOTA conjugate (e.g., 500 kD dextran-DOTA-Bn (Y), 500 kD dextran-DOTA-Bn (Lu), or 500 kD dextran-DOTA-Bn (In) etc.). In some embodiments, the clearing agent and the radiolabeled-DOTA hapten are administered without further administration of the anti-DOTA multi-specific antibody. For example, in some embodiments, an anti-DOTA multi-specific antibody is administered according to a regimen that includes at least one cycle of: (i) administration of the an anti-DOTA multi-specific antibody of the present technology (optionally so that relevant tumor cells are saturated); (ii) administration of a radiolabeled-DOTA hapten and, optionally a clearing agent; (iii) optional additional administration of the radiolabeled-DOTA hapten and/or the clearing agent, without additional administration of the anti-DOTA multi-specific antibody. In some embodiments, the method may comprise multiple such cycles (e.g., 1, 2, 3, 4, 5, 6, 7, 8, 9, 10 or more cycles).

Also provided herein are methods for treating cancer in a subject in need thereof comprising administering to the subject an effective amount of a complex comprising a radiolabeled-DOTA hapten and a multi-specific antibody of the present technology that recognizes and binds to the radiolabeled-DOTA hapten, a CD3 target antigen and a TSHR target antigen, wherein the complex is configured to localize to a tumor expressing the TSHR target antigen recognized by the multi-specific antibody of the complex. The therapeutic effectiveness of such a complex may be determined by computing the area under the curve (AUC) tumor: AUC normal tissue ratio. In some embodiments, the complex has a AUC tumor: AUC normal tissue ratio of about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Ex vivo armed T cells. In one aspect, the present disclosure provides an ex vivo armed T cell that is coated or complexed with an effective amount of an anti-TSHR multi-specific antibody of the present technology, wherein the anti-TSHR multi-specific antibody includes a CD3 binding domain (e.g., any and all embodiments of the OKT3 heavy chain immunoglobulin variable domain (V_{H}) and light chain immunoglobulin variable domain (V_{L}) disclosed herein), wherein the anti-TSHR multi-specific antibody is an immunoglobulin comprising two heavy chains and two light chains, wherein each of the light chains is fused to a single chain variable fragment (scFv). In some embodiments, at least one scFv of the anti-TSHR multi-specific antibody comprises the CD3 binding domain. Additionally or alternatively, in some embodiments, at least one scFv of the anti-TSHR multi-specific antibody comprises a DOTA binding domain (e.g., a C825 heavy chain immunoglobulin variable domain (V_{H}) and light chain immunoglobulin variable domain (V_{L})). Additionally or alternatively, in some embodiments, the ex vivo armed T cell is further coated or complexed with an effective amount of a multi-specific antibody that specifically binds to HER2 and T cells. In certain embodiments, the multi-specific antibody that specifically binds to HER2 and T cells comprises a light chain amino acid sequence of SEQ ID NO: 54 and a heavy chain amino acid sequence of SEQ ID NO: 56.

Also disclosed herein are methods for treating a TSHR-associated cancer in a subject in need thereof comprising administering to the subject an effective amount of any and all embodiments of the ex vivo armed T cell disclosed herein. In some embodiments, the ex vivo armed T cell is a αβ-T cell or a γδ-T cell. Additionally or alternatively, in some embodiments, the ex vivo armed T cell is obtained from a third party donor (e.g., allogeneic), or is obtained from the subject in need thereof (e.g., autologous). The ex vivo armed T cell may be cryopreserved or freshly harvested from a donor. Additionally or alternatively, in some embodiments, the TSHR-positive cancer is resistant to a RET inhibitor, a NTRK inhibitor, an ALK inhibitor, a RAF inhibitor, or a MEK kinase inhibitor.

Additionally or alternatively, in some embodiments, the methods of the present technology comprise administering to the subject an effective amount of a first population of ex vivo armed T cells that is coated or complexed with an effective amount of any and all embodiments of the anti-TSHR×CD3 multi-specific antibody disclosed herein and a second population of ex vivo armed T cells that is coated or complexed with an effective amount of a multi-specific antibody that specifically binds to HER2 and T cells, wherein the second population of ex vivo armed T cells is distinct from the first population of ex vivo armed T cells. In certain embodiments, the multi-specific antibody present on the second population of ex vivo armed T cells comprises a light chain amino acid sequence of SEQ ID NO: 54 and a heavy chain amino acid sequence of SEQ ID NO: 56.

Toxicity. Optimally, an effective amount (e.g., dose) of an anti-TSHR multi-specific antibody described herein will provide therapeutic benefit without causing substantial toxicity to the subject. Toxicity of the anti-TSHR multi-specific antibody described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the anti-TSHR multi-specific antibody described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

Formulations of Pharmaceutical Compositions. According to the methods of the present technology, the anti-TSHR multi-specific antibody can be incorporated into pharmaceutical compositions suitable for administration. The pharmaceutical compositions generally comprise recombinant or substantially purified antibody and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (See, e.g., Remington's Pharmaceutical Sciences, Mack Publishing Co., Easton, PA 18^{th} ed., 1990). The pharmaceutical compositions are generally formulated as sterile, substantially isotonic and in full compliance with all Good Manufacturing Practice (GMP) regulations of the U.S. Food and Drug Administration. The pharmaceutical composition may further comprise an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-TSHR multi-specific antibody, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-TSHR multi-specific antibody named in this technology can be present in unsalified or unesterified form, or in salified and/or esterified form, and the naming of such anti-TSHR multi-specific antibody is intended to include both the original (unsalified and unesterified) compound and its pharmaceutically-acceptable salts and esters. Also, certain embodiments of the present technology can be present in more than one stereoisomeric form, and the naming of such anti-TSHR multi-specific antibody is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the anti-TSHR multi-specific antibody, use thereof in the compositions is contemplated. Supplementary active compounds can also be incorporated into the compositions.

A pharmaceutical composition of the present technology is formulated to be compatible with its intended route of administration. The anti-TSHR multi-specific antibody compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The anti-TSHR multi-specific antibody can optionally be administered in combination with other agents that are at least partly effective in treating various TSHR-associated pathologies.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating an anti-TSHR multi-specific antibody of the present technology in the required amount in an appropriate solvent with one or a combination of ingredients enumerated above, as required, followed by filtered sterilization. Generally, dispersions are prepared by incorporating the anti-TSHR multi-specific antibody into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The antibodies of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the anti-TSHR multi-specific antibody can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-TSHR multi-specific antibody is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-TSHR multi-specific antibody is formulated into ointments, salves, gels, or creams as generally known in the art.

The anti-TSHR multi-specific antibody can also be prepared as pharmaceutical compositions in the form of suppositories (e.g., with conventional suppository bases such as cocoa butter and other glycerides) or retention enemas for rectal delivery.

In one embodiment, the anti-TSHR multi-specific antibody is prepared with carriers that will protect the anti-TSHR multi-specific antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Kits of the Present Technology

The present technology provides kits for the detection and/or treatment of TSHR-associated cancers, comprising at least one immunoglobulin-related composition of the present technology (e.g., any antibody or antigen binding fragment described herein), or a functional variant (e.g., substitutional variant) thereof. Optionally, the above described components of the kits of the present technology are packed in suitable containers and labeled for diagnosis and/or treatment of TSHR-associated cancers. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive TSHR protein in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more humanized, chimeric, bispecific, or multi-specific anti-TSHR antibodies of the present technology (or antigen binding fragments thereof) capable of binding a TSHR protein in a biological sample; means for determining the amount of the TSHR protein in the sample; and means for comparing the amount of the immunoreactive TSHR protein in the sample with a standard. One or more of the anti-TSHR antibodies may be labeled. The kit components, (e.g., reagents) can be packaged in a suitable container. The kit can further comprise instructions for using the kit to detect the immunoreactive TSHR protein.

For antibody-based kits, the kit can comprise, e.g., 1) a first antibody, e.g. a humanized, chimeric, bispecific, or multi-specific TSHR antibody of the present technology (or an antigen binding fragment thereof), attached to a solid support, which binds to a TSHR protein; and, optionally; 2) a second, different antibody which binds to either the TSHR protein or to the first antibody, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a TSHR protein in vitro or in vivo, or for treatment of TSHR-associated cancers in a subject in need thereof. In certain embodiments, the use of the reagents can be according to the methods of the present technology.

### EXAMPLES

The following examples are provided to further illustrate the methods of the present disclosure. These examples are illustrative only and are not intended to limit the scope of the disclosure in any way.

### Example 1: Materials and Methods

Cell Lines and culture conditions. Thyroid cancer cell lines were acquired directly from the originator or from repositories. The following RAS mutant thyroid cancer cell lines were used: ML1, ASH-3, EAM306, KMH-2, TT2609-C02, HTH83, HTH74, HTH104, JEM493, CAL-62, C-643 and ACT1. The following BRAF-mutant thyroid cancer cell lines were used: KTC1, BHT101, 8505C, T235, OCUT1, THJ560, SW1736. Additionally, the follicular thyroid cancer cell line FTC133, the medullary thyroid cancer cell line TT and HEK293T cells were used. All lines were maintained at 37° C. and 5% CO₂ in humidified atmosphere and were grown in the recommended media (RPMI-1640, DMEM or DMEM: RPMI) supplemented with 10% of FBS, 2 mmol/L glutamine, 50 U/mL penicillin, and 50 μg/mL streptomycin (GIBCO). All cell lines tested negative for mycoplasma and were authenticated using short tandem repeat and single-nucleotide polymorphism analyses.

Plasmids and Constructs. The human and mouse TSHR constructs (HA-TSHR, Hu-TSHR, mTshr) were obtained from Terry Davies (Mount Sinai, New York). TSHR-Tango and the human TSHR expression vector was obtained from Bryan Roth (Addgene plasmid #66518). The cDNA of human or mouse TSHR from any of these constructs were PCR amplified and subsequently cloned into the pL VX-puro vector (Clonetech) to generate pLVX-puro-HA-TSHR and pLVX-puro-TSHR-ires-GFP. All constructs were sequence-verified.

TSHR Overexpression in Cell Lines. To generate stable TSHR-expressing 8505C, THJ560 and BHT101 thyroid cancer cells, the pL VX-puro-TSHR-IRES-GFP or HA-TSHR constructs were used for lentiviral production in HEK293FT cells using the Mission Lentiviral Packing Mix (Sigma). Constitutively expressing 8505C-TSHR-IRES-GFP, THJ560-HA-TSHR and BHT101-HA-TSHR stable lines were generated by infecting cells with the corresponding viral particles in the presence of 8 μg/mL polybrene (Sigma) overnight. After 24 h in complete medium, cells were selected in 1 μg/mL puromycin. The mass culture was then used to establish clones derived from single cells, which were expanded and tested for the expression of TSHR by flow cytometry.

Flow Cytometry. Flow cytometry was performed to assess specific binding of TSHR and HER2 bispecific antibodies or to quantify their membrane expression. TSHR and HER2 BsAb specific binding was tested in HEK293T cells transfected with the indicated TSHR expression constructs using FuGENE HD (Promega). Induction of TSHR and HER2 expression was tested in a panel of RAS mutant thyroid cancer cell lines by treating them with DMSO or 20 nm of the MEK inhibitor trametinib for 48 and 72 h. The human monoclonal TSHR antibodies M22 and K1-70 (Kronus), or the bispecific antibodies M22-TSHR/CD3, K1-70-TSHR/CD3, or HER2/CD3 were used for these experiments. For flow analysis cells were harvested using 0.02% EDTA in phosphate-buffered saline (PBS). After 2 PBS washes cells were stained for 1 h at 4° C. either with control antibody (CD19) or the respective primary antibodies in PBS (10 μg/mL) followed by PE conjugated anti-human secondary antibody in PBS (0.5 μg/mL) (Southernbiotech, 2040-09) for 30 min at 4° C. After washing, cells were captured using a BD LSRFortessa (BD Biosciences). Cells stained with DAPI (1 μg/mL) were excluded. Cytometric analysis was performed using FlowJo (V10.6; FlowJo) to obtain median fluorescence intensity (MFI) or to quantify molecules per cell using QuantiBRITE PE reference beads (BD Biosciences).

Quantitative Real-Time PCR. Total RNA was isolated using the RNeasy Mini Kit (Qiagen; catalog no. 74104) with on-column DNase digestion according to the manufacturer's instructions. Total RNA (1 μg) was reverse-transcribed into cDNA using SuperScript III Reverse Transcriptase (Invitrogen) using random hexamers. qRT-PCR was carried out in triplicate using Power SYBR Green PCR Master Mix (Applied Biosystems) on the ViiA 7 Real-Time PCR System (Applied Biosystems). Relative TSHR mRNA quantification was assessed using TSHR specific primer sets and β-actin served as the endogenous normalization control.

cAMP assay. TSHR-expressing 8505C-TSHR-IRES-GFP cells were seeded into 96-well plates (5×10⁴ per well). After cells adhered, cells were washed three times and the culture medium was replaced with serum free media in the presence of 1 mM IBMX (Sigma) and incubated at 37° C. overnight in a humidified incubator. Cells were then incubated for 30 min with either bovine TSH (National Hormone & Peptide Program, LLC), M22 and K1-70 BsAb. Cells were lysed using assay lysis buffer, and cAMP measured with the CAMP-Screen Direct system (Applied Biosystems, Foster City, CA) following the manufacturer's protocol.

T-cell isolation and activation ex vivo. Isolation of naïve T-cells, expansion and activation ex vivo was performed as previously described in Park, J. A., et al., Journal for Immuno Therapy of Cancer, 9 (5): p. e002222 (2021). Briefly, peripheral blood mononuclear cells (PBMCs) were isolated from buffy coats (New York Blood Center) using Ficoll. Naïve T-cells were purified using Pan T Cell Isolation Kit (Miltenyi Biotec) and T-cells expanded by CD3/CD28 Dynabeads (Gibco) for 7-14 days in the presence of 30IU/mL of interleukin-2 (IL-2). T-cells were then analyzed for proportion of CD3(+), CD4(+) and CD8(+) T cells, and the fraction of CD4 or CD8 T cells was maintained between 40% and 60%. These activated T cells were used for all experiments involving T-cells.

In vitro T-cell cytotoxicity assay. The T cell cytotoxicity mediated by the M22/CD3 and K1-70/CD3 BsAb was tested in a panel of thyroid cancer cell lines treated with or without trametinib. Cells were pretreatment with 20 nm trametinib for 72 h followed by assaying for antibody-dependent T cell-mediated cytotoxicity (ADTC). The ADTC assay was performed by determination of ⁵¹Cr release (Xu, H., et al., Cancer Immunol Res, 3 (3): p. 266-77 (2015)) and the percent specific lysis obtained was used to calculate EC50 using GraphPad-Prism (version 10.0; GraphPad Software, Inc.). Briefly, cells were labeled with sodium ⁵¹Cr chromate (⁵¹CrNa2CrO4; Amersham, Arlington Heights, Illinois) at a concentration of 100 μCi/10⁶ cells at 37° C. for 1 h. After two washings, radiolabeled cells were seeded into 96-well U bottom plates. BsAb and T cells were added to the target cells at a concentration of 10:1 (E:T). After incubation at 37° C. for 4 h, the ⁵¹Cr released into the supernatant was measured by gamma counter (Packed Instrument, Downers Grove, Illinois). The percentage lysis and antibody mediated specific lysis was calculated as follows (cpm=counts per minute of ⁵¹Cr release):

$$\% Lysis = \frac{( sample well cpm - background cpm )}{( total cpm - background cpm )} \times 100$$

$$\% Specific Lysis = lysis of BsAb sample - lysis of control sample$$

Total cpm was assessed by lysis with 100 μl of 10% sodium dodecyl sulfate (Sigma) and background cpm was measured in the absence of T-cells and BsAb. All sample wells were seeded and measured in triplicate.

Thyroid Ablation. To prevent competition for binding with the TSHR BsAb treatment, thyroid ablation was performed in mice using 131-iodine (RAI) following a low-iodine diet (LID) as previously described in Oh, J. M. et al., Scientific Reports, 7 (1): p. 13284 (2017). Briefly, mice were fed with LID for 7d and treated with 2.8 MBq of ¹³¹I via IP injections followed by L-T4 supplement in drinking water to suppress TSH till the end of the BsAb treatment experiments. Ablation of the mouse thyroid was confirmed by SPECT scanning of the thyroid gland using ^{99m}Tc pertechnetate, whose uptake in the thyroid was significantly decreased 2-3 weeks after RAI treatment.

In vivo efficacy of TSHR and HER2 BsAb. The in vivo anti-tumor effects of TSHR- and HER2-BsAb were tested with or without co-treatment with the MEK inhibitor trametinib in ML-1 cells. ML-1 cells resuspended in 50% Matrigel (Corning) were implanted (0.5×10⁶ cells/mice) into the flank of 6-10 week-old female BALB-Rag2−/−IL-2R-γc-KO (BRG) mice (Taconic Biosciences). Prior to tumor cell injection, the TSHR-BsAb cohort mice were subjected to thyroid ablation as described above. Treatments were initiated after tumors were established, with an average tumor volume of 150 mm³ as estimated by measuring the length and width with calipers (width²×length×0.52). Tumor bearing mice were randomly assigned into the respective treatment arms for TSHR and HER2-BsAb groups. Treatment arms for TSHR-BsAb group: Tumor cells only (No treatment), T cell only, T cell+CD19 BsAb, T cell+M22 or K1-70-BsAb, T cell+CD19+trametinib and T cell+M22 or K1-70-BsAb+trametinib. Treatment arms for HER2-BsAb group: Tumor only, T cell only, T cell+CD19 BsAb, T cell+HER2-BsAb, T cell+CD19 BsAb+trametinib and T cell+HER2-BsAb+trametinib. TSHR, HER2, and control (CD19) bispecific antibodies (3 or 10 μg) along with T cells (2×10⁷ activated T-cells) were administered twice a week for 3 weeks (total 6 doses). In subsets with and without BsAb, trametinib (3 mg/kg or vehicle: 5% HPMC+10% Tween80; Selleckchem), was administered by oral gavage for 3 weeks. Mice were weighed at the start of treatment and every second day during the treatment period. Tumor volume was measured every 2 to 3 days with calipers. After treatment, mice were humanely killed, and dissected tumors were fixed for IHC, flash frozen for RNA/Protein analysis and dissociated into single cell suspensions for flow cytometry. All animal experiments were repeated with at least two different donor T cells. All animal experiments were performed in accordance with a protocol approved by the Institutional Animal Care and Use of Committee of MSKCC.

Immunohistochemical staining. Paraformaldehyde (PFA)-fixed paraffin-embedded xenograft tissues were sectioned and tested for infiltration of T cells using immunohistochemical staining of human CD3 T cells performed by the Molecular Cytology Core Facility of MSKCC using Discovery XT processor (Ventana Medical Systems). CD3 stained slides were scanned with Panoramic P250 Flash scanner (3-DHistech) using 20×/0.8NA objective lens. Regions of interest around the tissues were then drawn and exported as .tif files using Caseviewer (3-DHistech).

T cell trafficking: Bioluminescence imaging was performed to monitor T-cell trafficking after treatment with bispecific antibodies with and without trametinib. BRG mice aged 6-10 weeks were subcutaneously implanted with ML1 cells, and trametinib treatment was initiated in the respective group after tumors were established. After 2 days of trametinib pre-treatment, luciferase positive T-cells armed with CD19 (control) or TSHR or HER2 BsAb were injected and monitored 24 h later by intravenous injection of 2 mg D-luciferin (PerkinElmer). Bioluminescence images were acquired with an IVIS Spectrum In Vivo Imaging System (Caliper Life Sciences) and images analyzed using Living Image software (V.2.60; Xenogen).

Statistical Analysis. The statistical software GraphPad-Prism (version 10.0; GraphPad Software, Inc.) was used to analyze the data. All data for qRT-PCR/RNA-seq expression values, flow data, cAMP assay values and in vivo tumor growth were represented as mean±SEM, and P values were calculated using unpaired two-tailed Student t tests. P value of <0.05 was considered statistically significant.

### Example 2: Expression of TSHR in Thyroid Cancers and Normal Tissues

Thyroid cancers driven by BRAF^{V600E} and many of the cancers driven by oncogenic RAS or class II/III mutant BRAF are refractory to radioiodine therapy because constitutive MAPK pathway activation silences expression of thyroid differentiation genes, including TSHR, which is required for TSH-induction of key genes required for iodine incorporation and thyroid hormone biosynthesis (Chakravarty D et al., J. Clin. Invest 121:4700-4711 (2011)). This is a reversible process, in that RAF or MEK inhibitors restore TSHR gene expression and thyroid differentiated function in BRAF or RAS-driven thyroid cells in vitro (Knauf J A et al., Oncogene 22:4406-4412 (2003)), in genetically engineered mouse models (Nagarajah J et al., J. Clin. Invest 126:4119-4124 (2016); Saqcena M et al., Cancer Discov 11:1158-1175 (2021)) and in patients (Ho A L et al., N. Engl. J. Med 368:623-632 (2013); Dunn L A et al., J Clin Endocrinol Metab 104:1417-1428 (2019); Rothenberg S M et al., Clin Cancer Res 21:1028-35 (2015)).

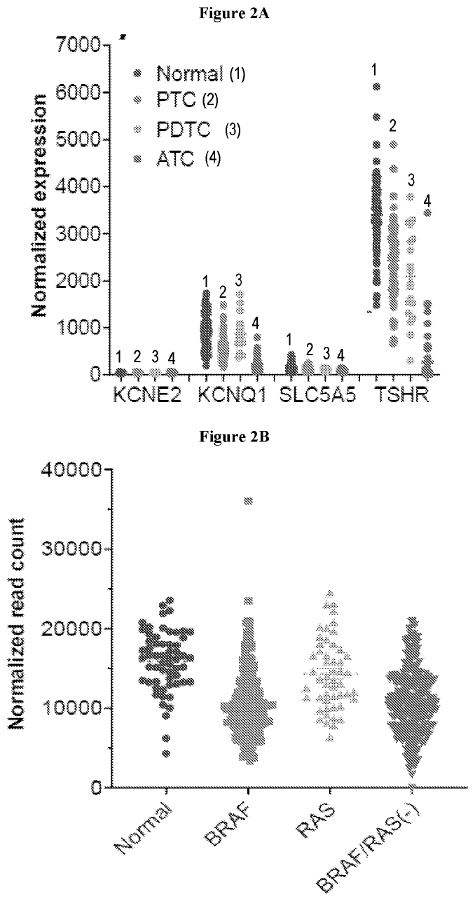

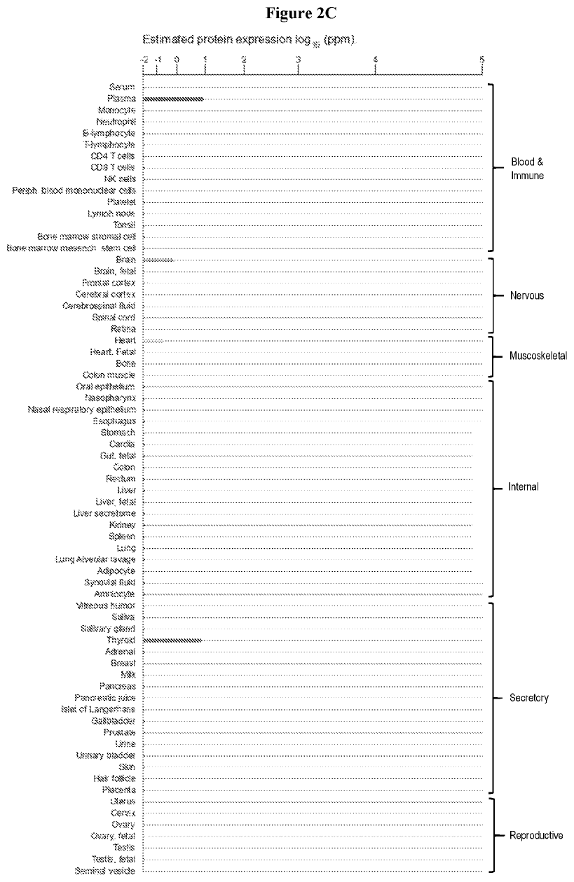

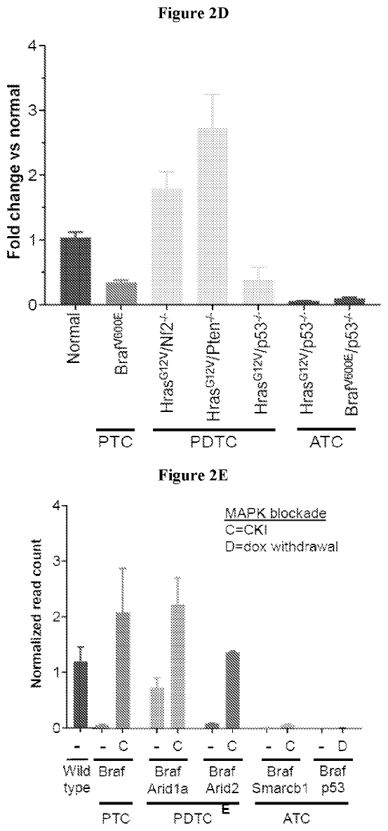

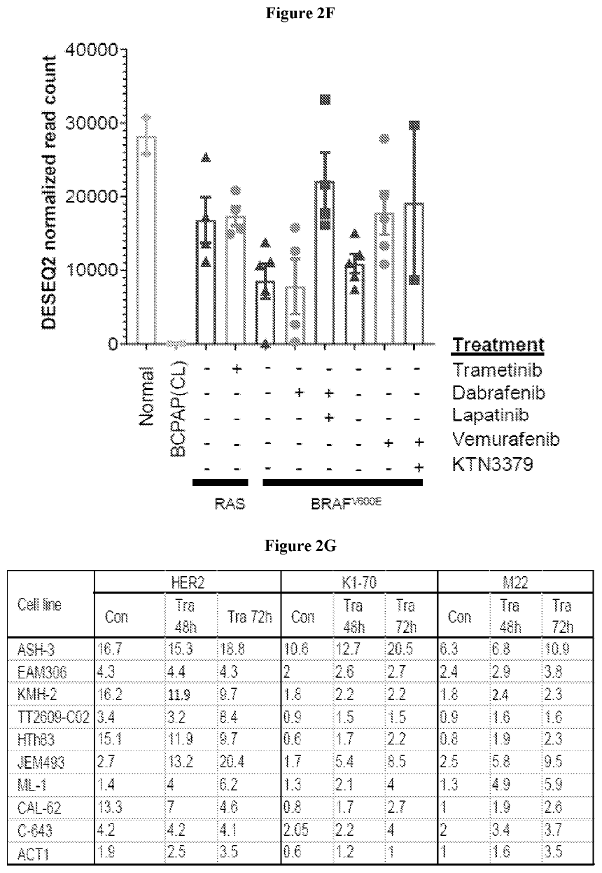

TSHR is the highest expressing basolateral membrane protein in thyroid cancer patients (FIGS. 2A-2B), and the expression is highly tissue restricted (FIG. 2C). Blocking MAPK fully restored TSHR mRNA to wild type levels or higher in BrafV600E-driven murine PTCs and PDTCs, but not ATCs (FIGS. 2D-2E). TSHR mRNA levels in lesional biopsies of patients with BRAF^{V600E}-RAIR-thyroid cancer treated with RAF and HER kinase inhibitors were restored to ˜90% of levels in normal thyroid (FIG. 2F). By contrast with BRAF^{V600E}-RAIR tumors, most of the RAS mutant cancers had TSHR levels similar to normal thyroid. However, in the RAS mutant cancers with lower baseline TSHR levels, the MEK inhibitor trametinib increased TSHR expression. To explore the adaptive response to MAPK inhibition in RAS mutant thyroid cancers, a panel of RAS-mutant thyroid cancer cell lines (n=10) including anaplastic thyroid cancer cells were screened demonstrating HER2 and TSHR plasma membrane expression in all lines tested at 48 and/or 72 hr post treatment with trametinib (FIG. 2G) as assessed by flow, and at the level of mRNA in a representative KRAS mutant ML1 cell line (FIG. 2H).

### Example 3: Generation and In Vitro Characterization of Anti-TSHR T-Cell Engaging BsAbs of the Present Technology

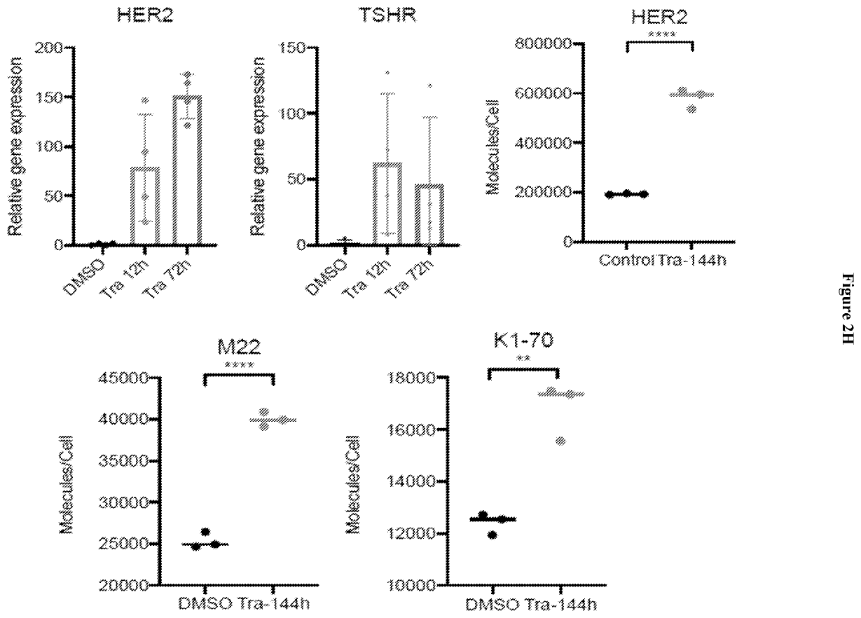

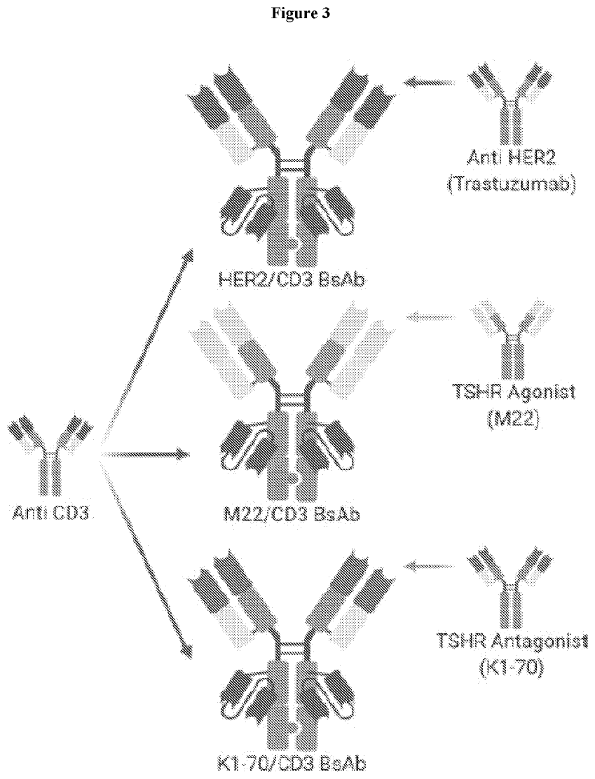

TSHR T-cell engaging BsAbs (TSHR T-BsAbs) using the IgG-[L]-scFv format were developed by swapping in the anti-TSHR IgG sequences corresponding to the M22 TSHR agonist monoclonal antibody or the K1-70 TSHR antagonist antibody. See FIG. 3.

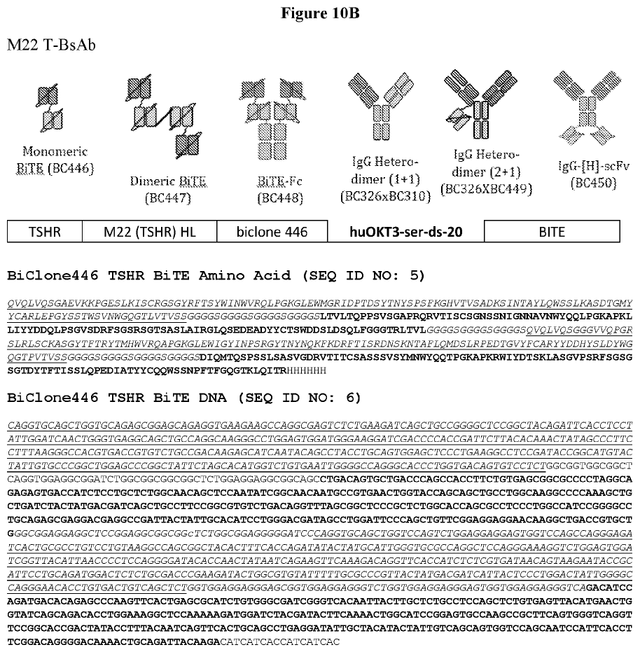

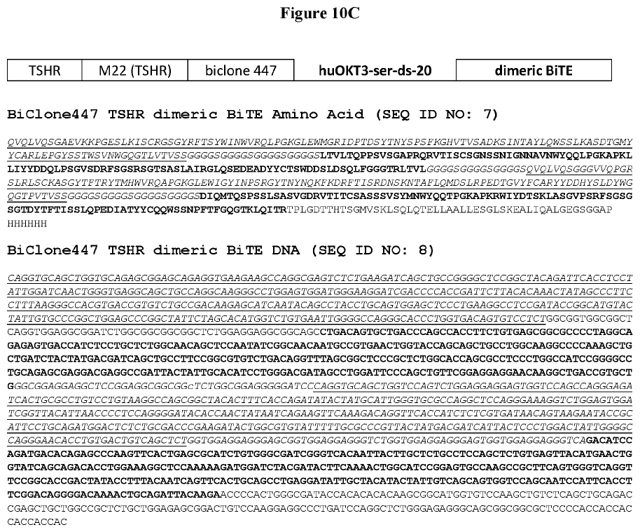

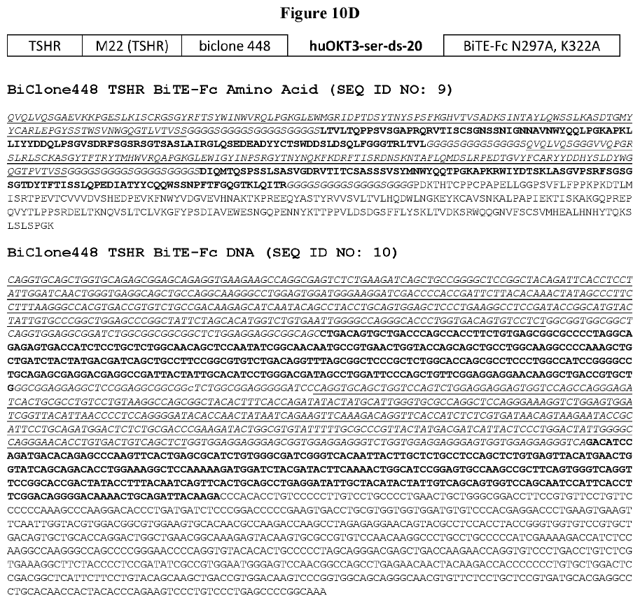

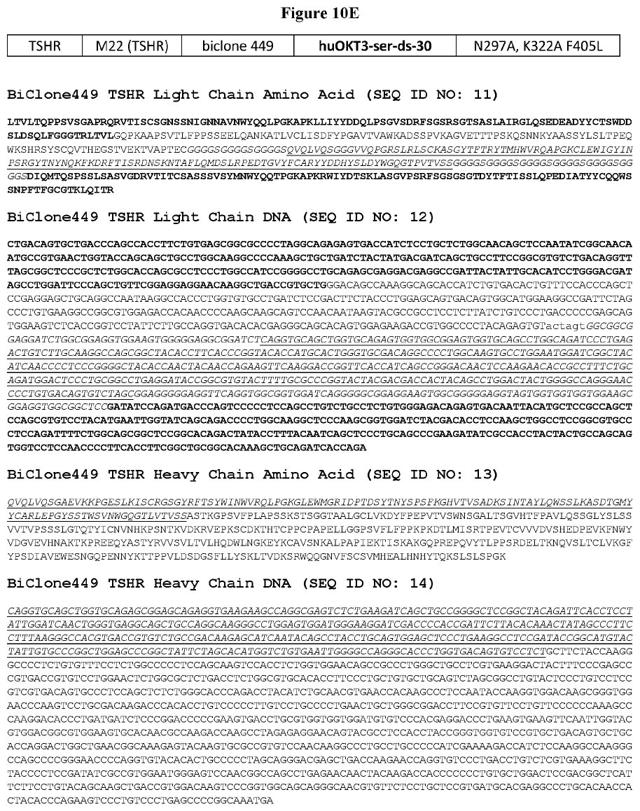

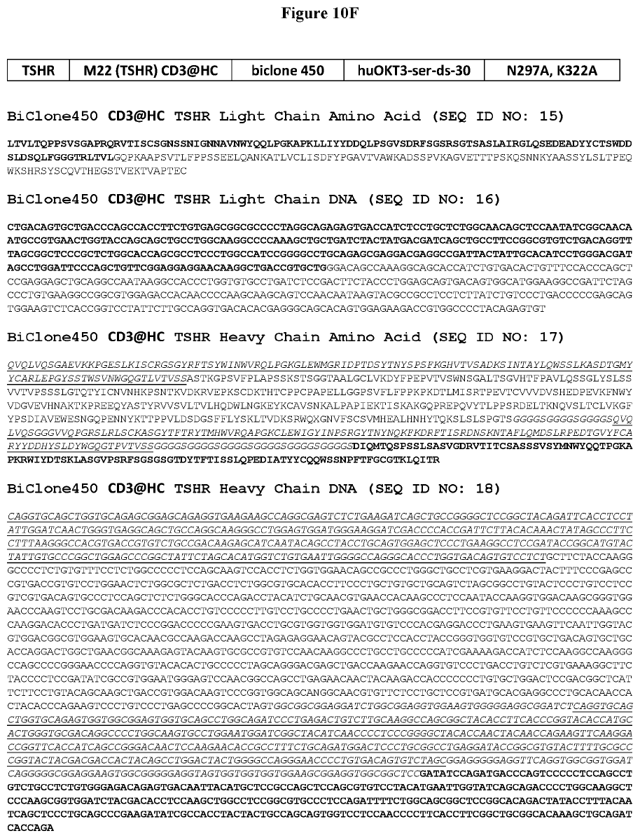

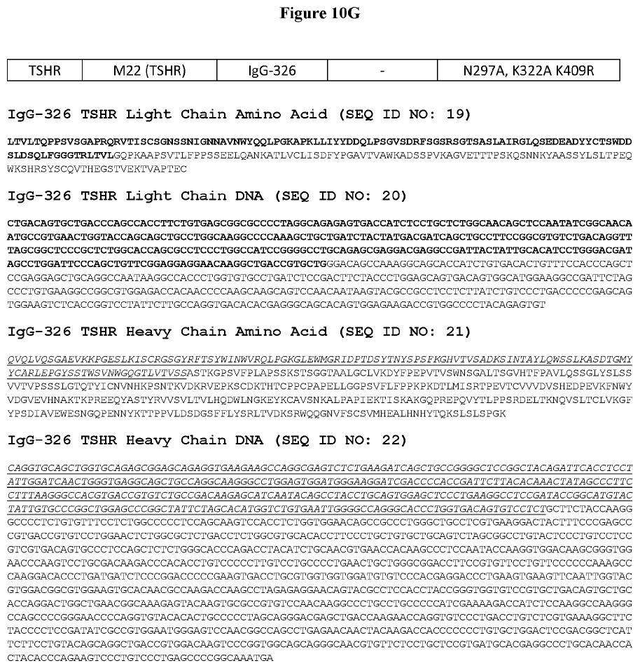

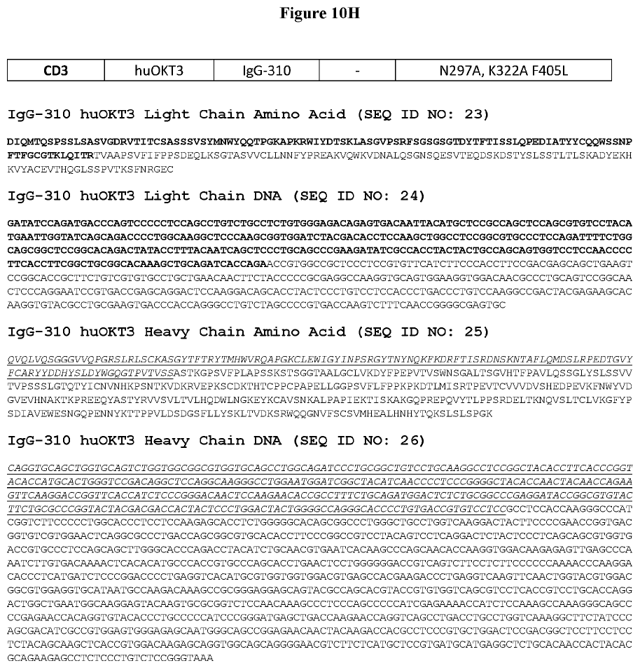

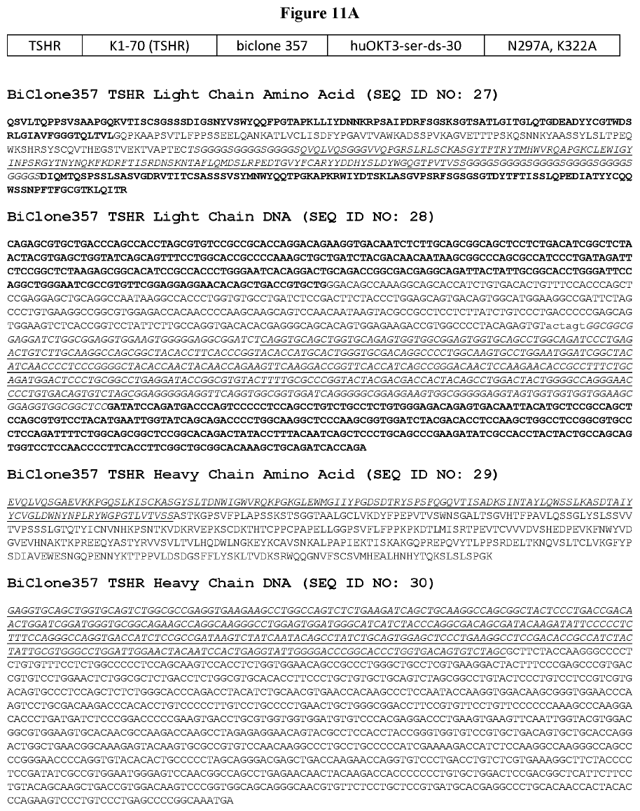

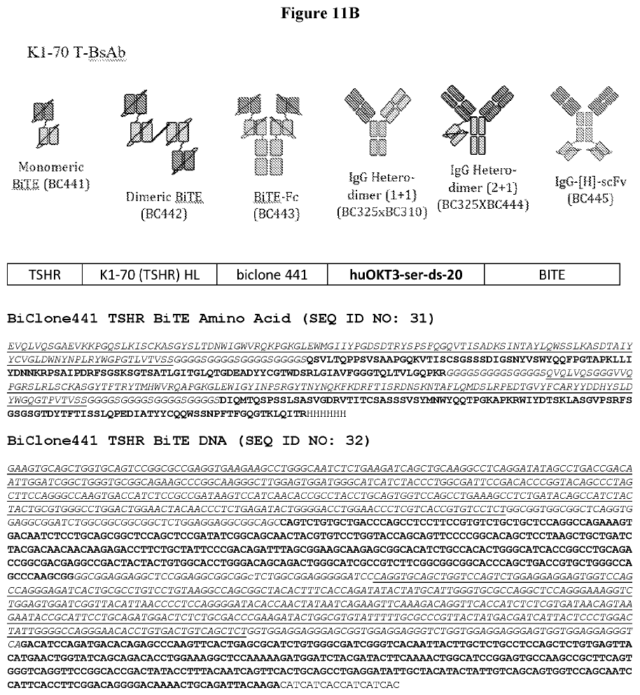

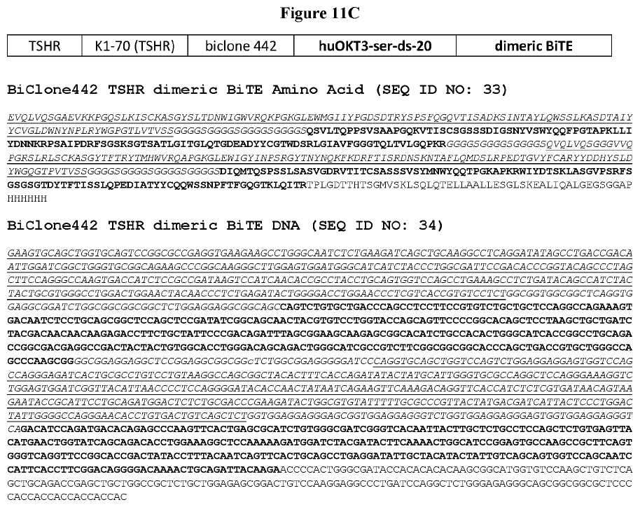

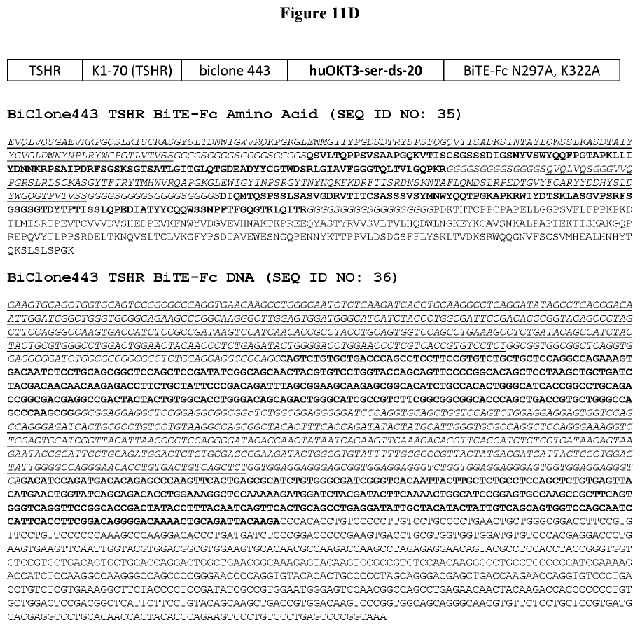

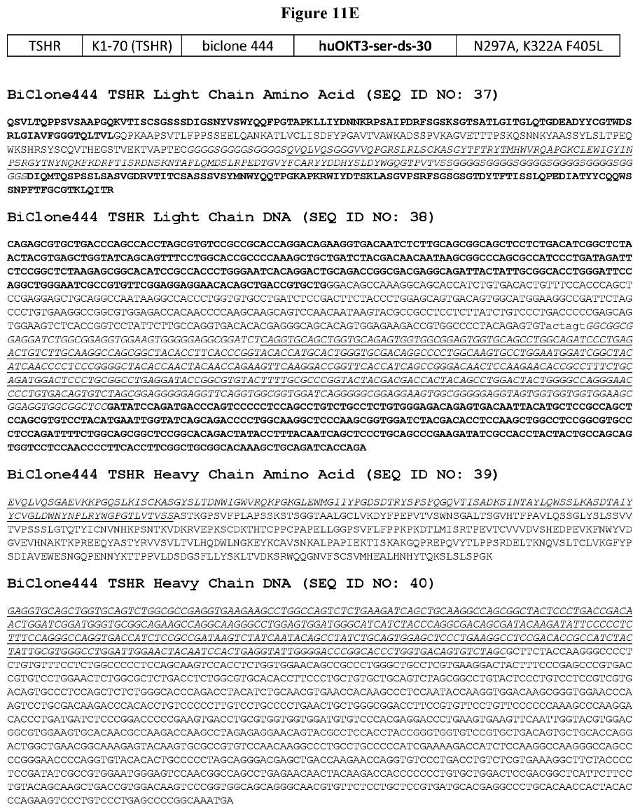

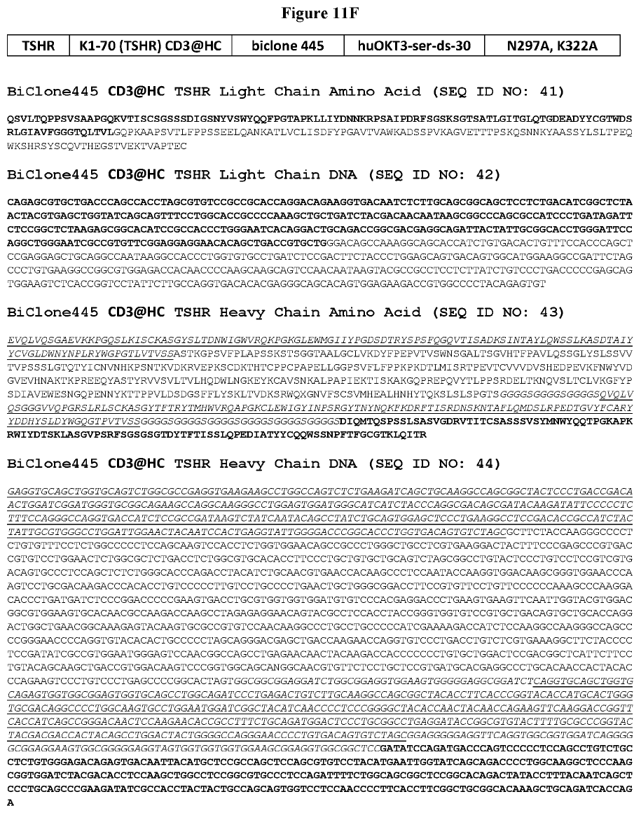

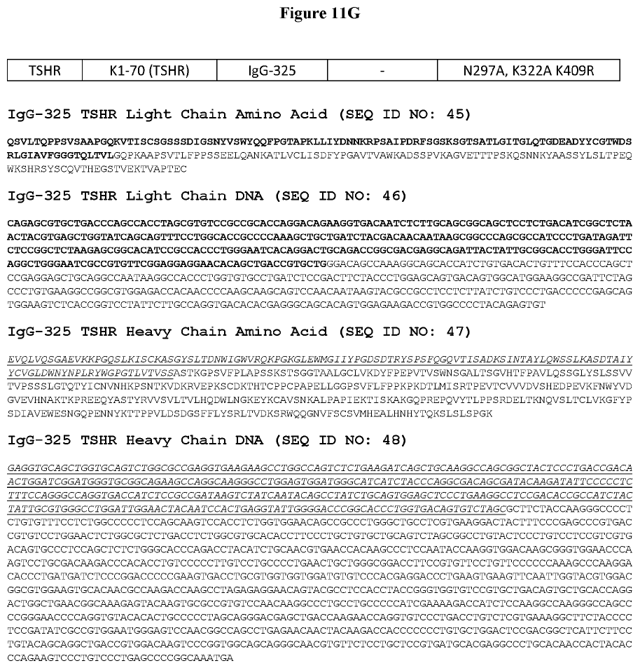

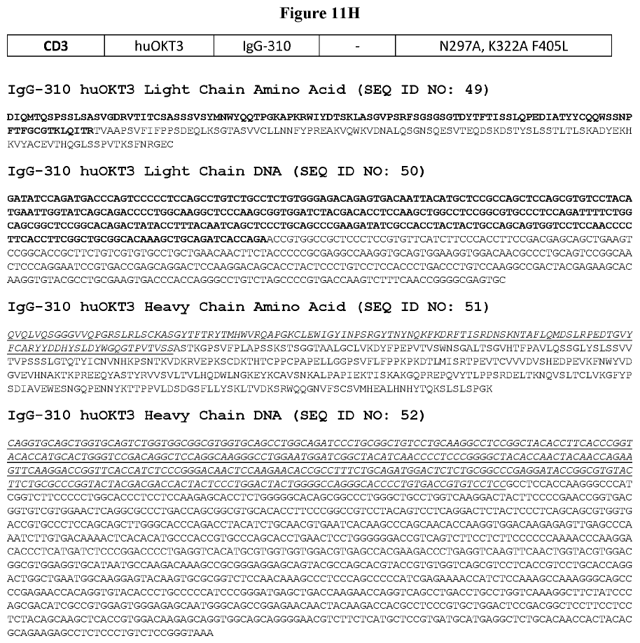

Both M22 (agonist) and K1-70 (antagonist) antibodies were selected given their unique properties: (1) human derived, hence low immunogenicity, (2) high affinity for TSHR (5×10¹⁰ & 4×10¹⁰ L/mol, respectively) (Li H et al., medRxiv: 2021.05.15.21256466 (2021); Oh J M et al., Sci Rep 7:13284 (2017)) and (3) known cocrystal structures, specificity and structure-function relationships. The M22/CD3 and K1-70/CD3 T-BsAb using the IgG-[L]-scFv format were generated by swapping in the anti-TSHR IgG sequences (FIGS. 10-11), and transfecting into Expi-CHO cells for transient expression or clonal cell line production. TSHR T-BsAbs were purified by protein A affinity chromatography. SEC-HPLC was used to assure purity of >90% monomer, stability at 37° C. or 40° C. and the absence of endotoxin.

### Example 4: Specific Binding and Functional Activity of Anti-TSHR-BsAbs of the Present Technology

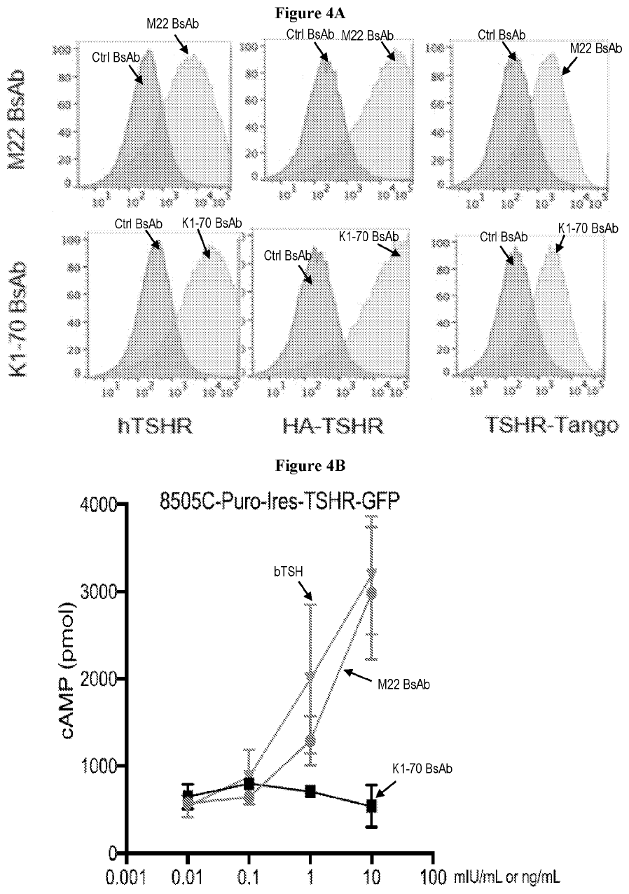

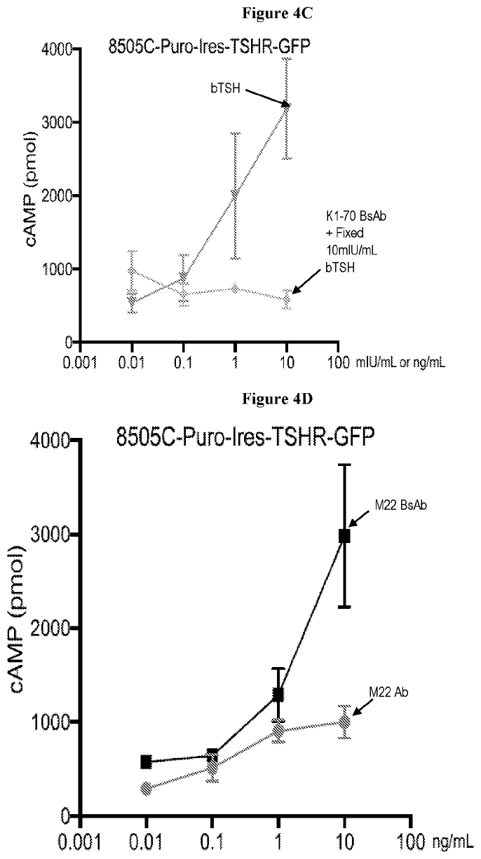

Specific binding and respective functional activities of TSHR T-BsAbs were evaluated by assaying their effects on TSHR-adenylyl cyclase induction of cyclic AMP (cAMP) in 8505C or 293T cells expressing human TSHR (FIG. 4A). M22/CD3 induced CAMP levels equivalent to that of bovine TSH (FIG. 4B), and K1-70/CD3 completely blocked TSH-stimulated cAMP (FIG. 4C). When compared to the respective parental antibody, M22/CD3 show increased ability to induce cAMP (FIG. 4D).

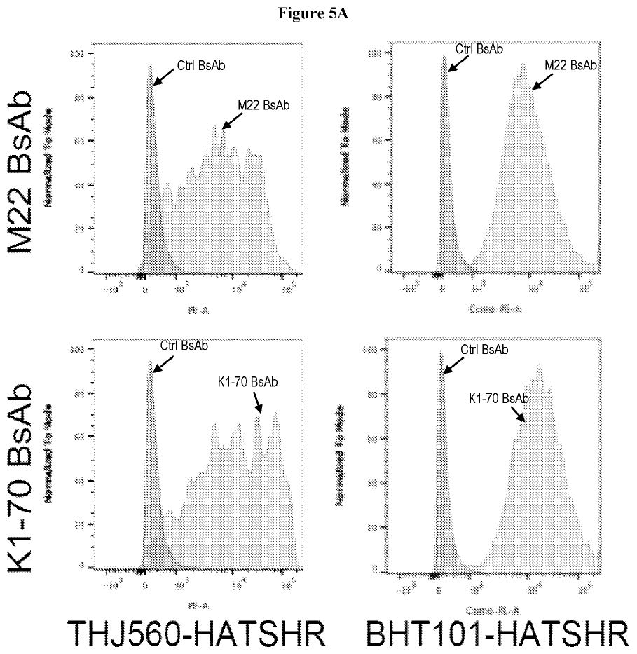

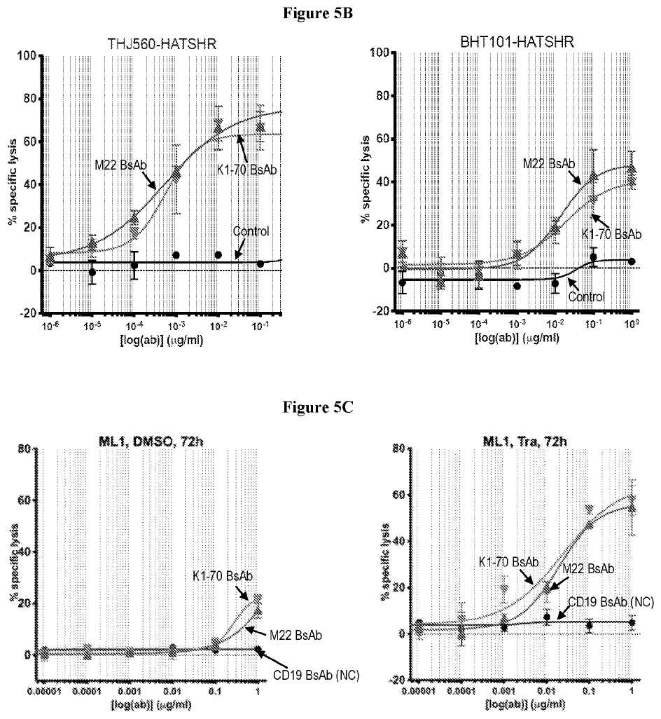

The TSHR T-BsAbs disclosed herein directed antibody dependent T cell mediated cytotoxicity (ADTC) in 2 thyroid cancer cell lines stably transduced with a TSHR expression vector (FIG. 5A), and showed comparable T-cell killing efficiency in vitro (FIG. 5B) as assessed by 51Cr release from tumor cells, which correlates with antibody binding. Additionally, M22/CD3 and K1-70/CD3 T cell cytotoxicity was tested on ML-1 cells with or without trametinib, which induces TSHR expression in this KRAS-mutant cell line. Accordingly, trametinib plus TSHR/CD3 T-BsAbs showed increased ADTC potency compared to DMSO (FIG. 5C).

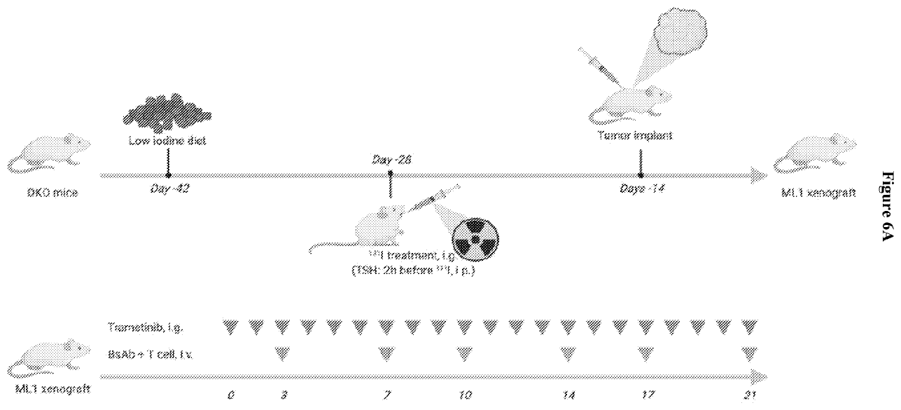

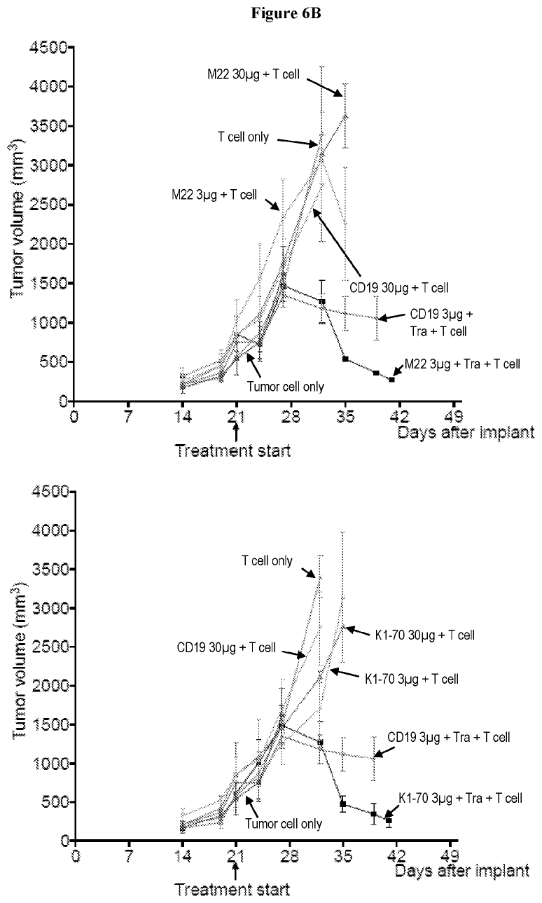

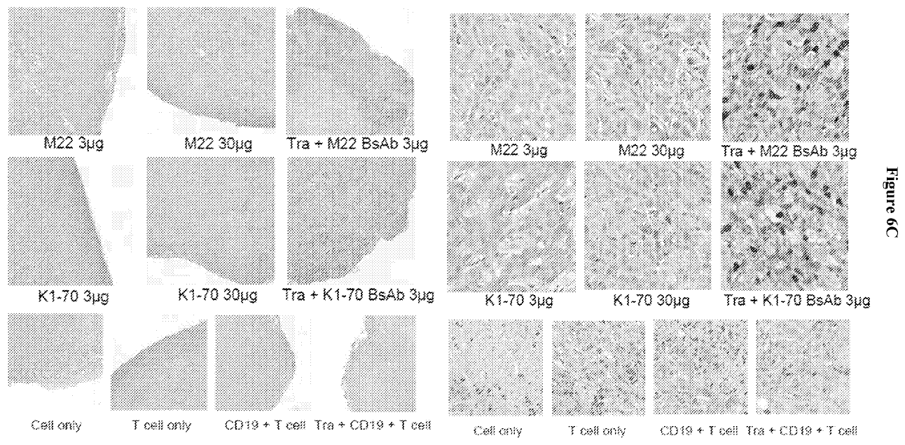

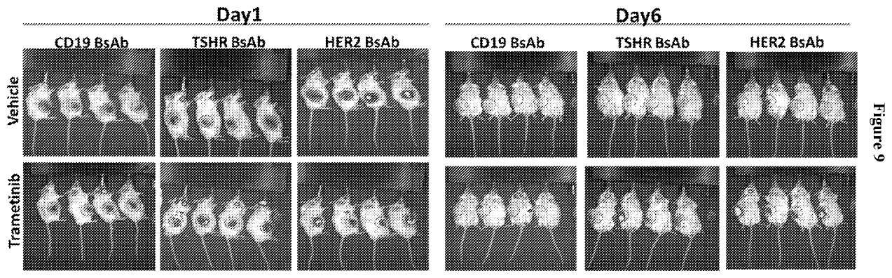

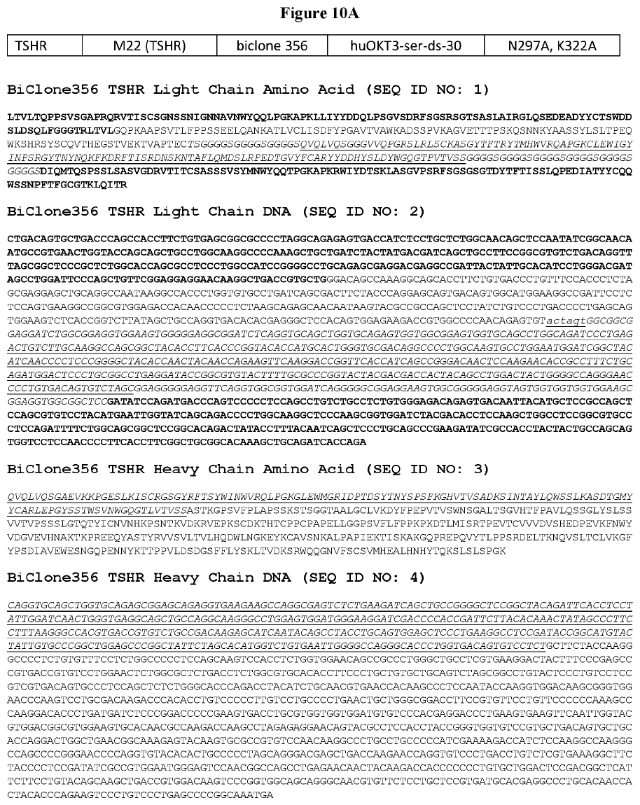

To determine in vivo efficacy of the TSHR-T-BsAb with and without trametinib, the thyroid cancer cell line ML-1 was implanted into the flanks of BRG mice. Prior to tumor cell injection, mice were fed with low iodide diet for 7d and treated with 2.8 mBq of ¹³¹I to ablate the normal thyroid to prevent competition for binding with the TSHR T-BsAb. Mice were also treated with levothyroxine (L-T4) to suppress TSH levels, for the same reason (FIG. 6A). This experiment showed a dramatic tumor response by the co-treatment of trametinib+TSHR T-BsAb compared to either T-BsAb alone (FIG. 6B), suggesting that MAPK inhibition induced-TSHR expression evokes an increased T-cell mediated anti-tumor response. Accordingly, enhanced CD3 staining was observed in the xenograft tissues from the combination therapy compared to the respective monotherapies, confirming higher T-cell infiltration (FIG. 6C). FIG. 9 shows higher T cell trafficking into tumors from mice receiving TSHR BsAb in the presence of trametinib.

### Example 5: In Vitro and In Vivo Cytotoxiciy of HER2×CD3 BsAbs

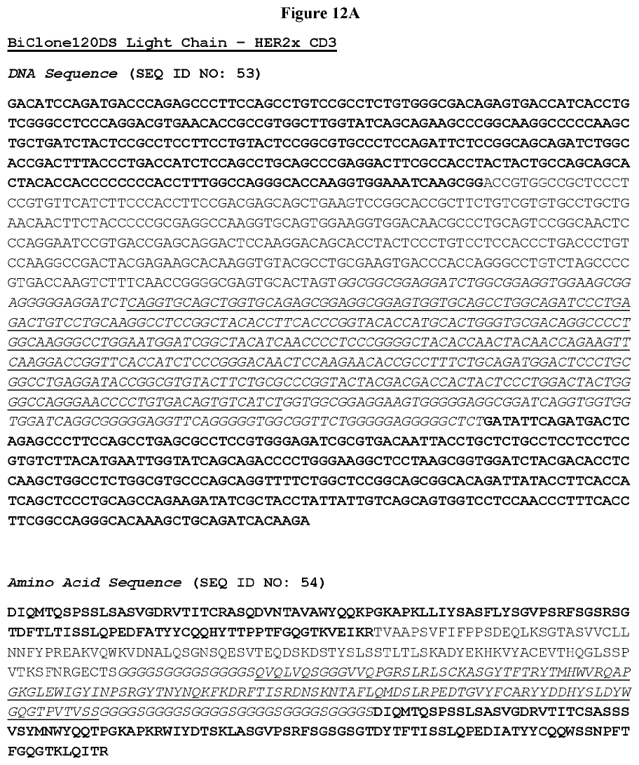

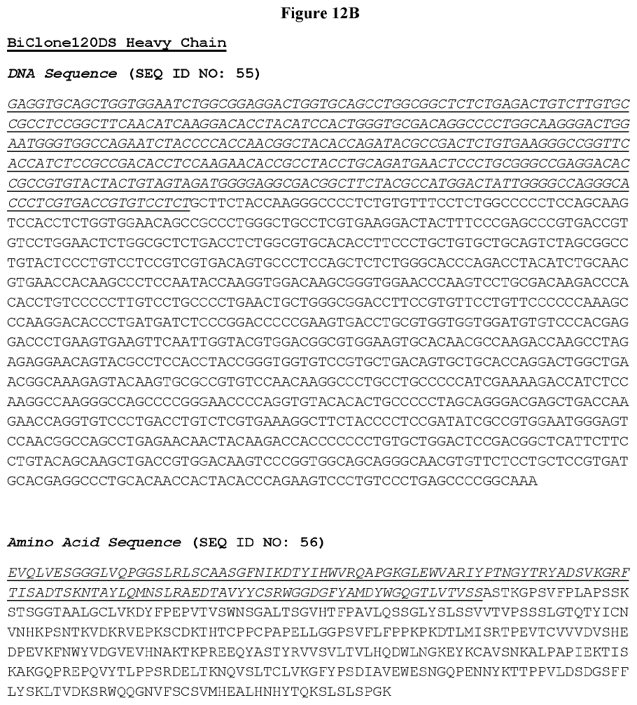

Within a given BRAF or RAS-mutant thyroid cancer exposed to RAF and/or MEK inhibitors, some cells may redifferentiate and increase membrane TSHR, whereas others may increase expression of HER2, or may express both TSHR and HER2. Without wishing to be bound by theory, it was anticipated that the induction of HER2 by RAF and/or MEK inhibitors presented a tractable target for a T cell-engaging bispecific antibody to enhance immune cell killing to HER2-expressing thyroid cancer cells and promote more profound and durable responses. Here, an anti-CD3 scFv (huOKT3) is attached to the carboxyl end of the light chain of the anti-HER2 antibody trastuzumab, whose Fc is silenced by N297A and K322A mutations to remove FcR binding and complement activation (see FIGS. 12A-12B).

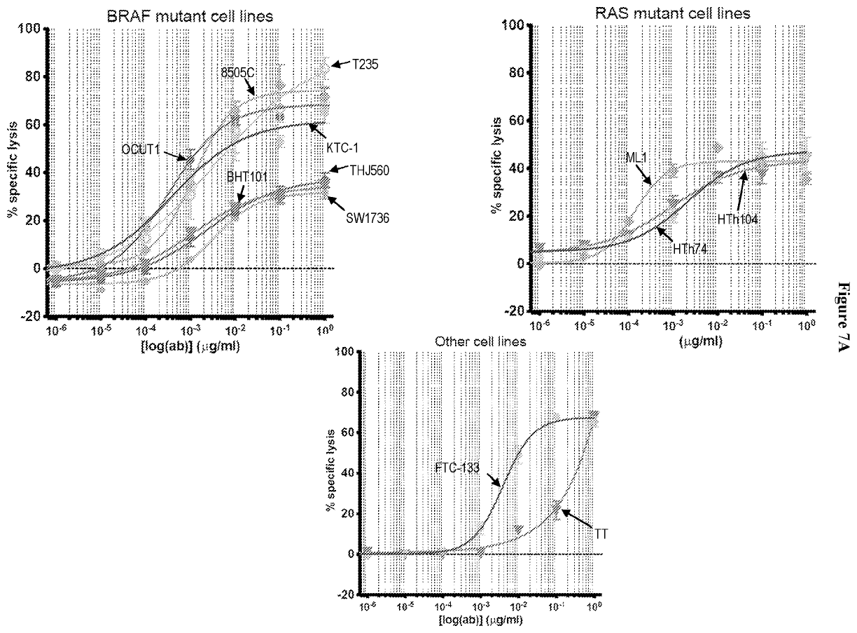

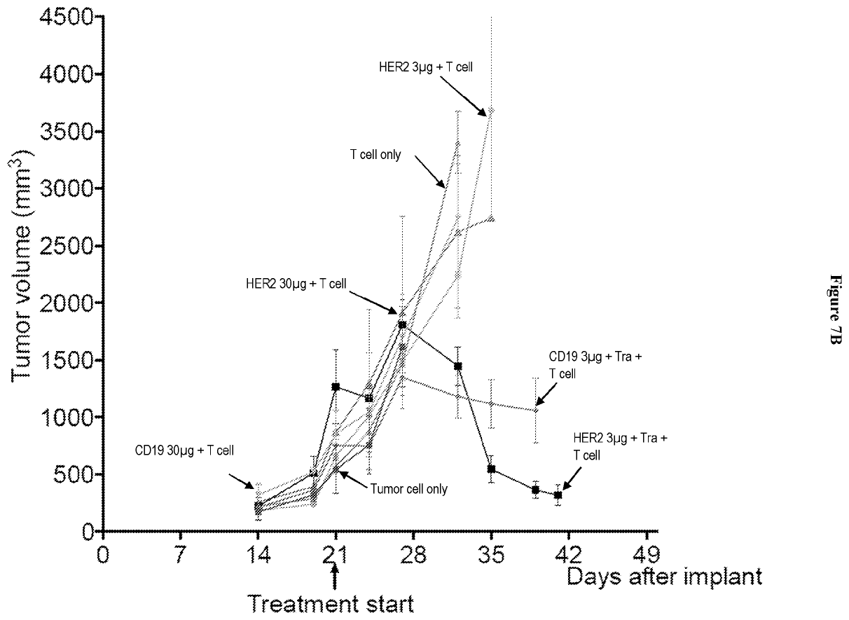

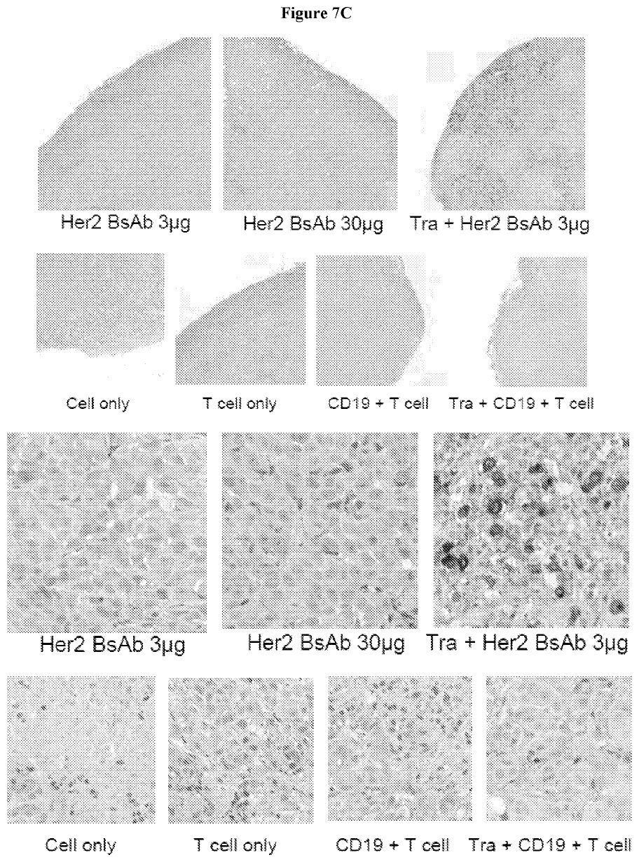

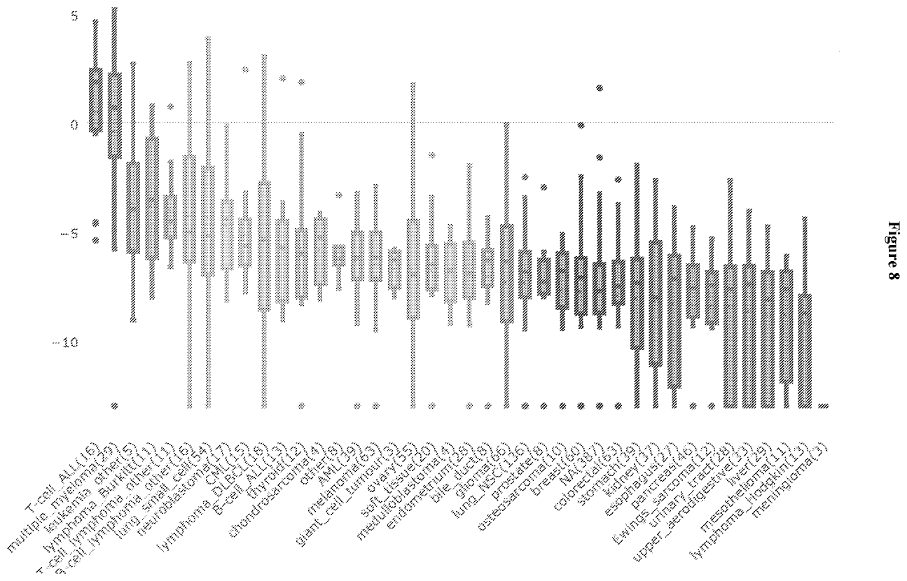

The increase in HER2 levels seen in both RAS and BRAF mutant thyroid cancers after inhibition of the MAPK pathway increases binding of the HER2 T-BsAb. In the presence of the MEK inhibitor trametinib, HER2 T-BsAb induced cell lysis upon addition of T cells in vitro, as assessed by ⁵¹Cr release in a panel of thyroid cancer cell lines (FIG. 7A). Accordingly, Trametinib+HER2 T-BsAb combination induced a near complete tumor regression in vivo (FIG. 7B), which was not seen with either single agent, and which correlated with enhanced T-cell infiltration as assessed by CD3 staining (FIG. 7C). FIG. 9 shows higher T cell trafficking into tumors from mice receiving HER2 BsAb in the presence of trametinib.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain are covalently bonded to one another, and wherein:
(a) each of the first polypeptide chain and the fourth polypeptide chain comprises in the N-terminal to C-terminal direction:
(i) a light chain variable domain of a first immunoglobulin that is capable of specifically binding to a first epitope;
(ii) a light chain constant domain of the first immunoglobulin;
(iii) a flexible peptide linker comprising the amino acid sequence (GGGGS)₃ (SEQ ID NO: 76); and
(iv) a light chain variable domain of a second immunoglobulin that is linked to a complementary heavy chain variable domain of the second immunoglobulin, or a heavy chain variable domain of a second immunoglobulin that is linked to a complementary light chain variable domain of the second immunoglobulin, wherein the light chain and heavy chain variable domains of the second immunoglobulin are capable of specifically binding to a second epitope, and are linked together via a flexible peptide linker comprising the amino acid sequence (GGGGS)₆ (SEQ ID NO: 77) to form a single-chain variable fragment; and
(b) each of the second polypeptide chain and the third polypeptide chain comprises in the N-terminal to C-terminal direction:
(i) a heavy chain variable domain of the first immunoglobulin that is capable of specifically binding to the first epitope; and
(ii) a heavy chain constant domain of the first immunoglobulin; and
wherein the heavy chain variable domain of the first immunoglobulin is SEQ ID NO: 57 or SEQ ID NO: 59, wherein the light chain variable domain of the first immunoglobulin is SEQ ID NO: 58 or SEQ ID NO: 60, wherein the heavy chain variable domain of the second immunoglobulin is SEQ ID NO: 61 or SEQ ID NO: 62 and wherein the light chain variable domain of the second immunoglobulin is SEQ ID NO: 63 or SEQ ID NO: 64.

2. The multi-specific antibody of claim 1, wherein each of the first polypeptide chain and the fourth polypeptide chain comprises the amino acid sequence of SEQ ID NO: 1, and wherein each of the second polypeptide chain and the third polypeptide chain comprises the amino acid sequence of SEQ ID NO: 3; or
wherein each of the first polypeptide chain and the fourth polypeptide chain comprises the amino acid sequence of SEQ ID NO: 27, and wherein each of the second polypeptide chain and the third polypeptide chain comprises the amino acid sequence of SEQ ID NO: 29.

3. (canceled)

4. A heterodimeric multi-specific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
(a) the first polypeptide chain comprises in the N-terminal to C-terminal direction:
(i) a light chain variable domain of a first immunoglobulin (VL-1) that is capable of specifically binding to a first epitope;
(ii) a light chain constant domain of the first immunoglobulin (CL-1);
(iii) a flexible peptide linker comprising the amino acid sequence (GGGGS)₃ (SEQ ID NO: 76); and
(iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 77) to form a single-chain variable fragment;
(b) the second polypeptide chain comprises in the N-terminal to C-terminal direction:
(i) a heavy chain variable domain of the first immunoglobulin (VH-1) that is capable of specifically binding to the first epitope;
(ii) a first CH1 domain of the first immunoglobulin (CH1-1); and
(iii) a first heterodimerization domain of the first immunoglobulin, wherein the first heterodimerization domain is incapable of forming a stable homodimer with another first heterodimerization domain;
(c) the third polypeptide chain comprises in the N-terminal to C-terminal direction:
(i) a heavy chain variable domain of a third immunoglobulin (VH-3) that is capable of specifically binding to a third epitope;
(ii) a second CH1 domain of the third immunoglobulin (CH1-3); and
(iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
(d) the fourth polypeptide chain comprises in the N-terminal to C-terminal direction:
(i) a light chain variable domain of the third immunoglobulin (VL-3) that is capable of specifically binding to the third epitope;
(ii) a light chain constant domain of the third immunoglobulin (CL-3);
(iii) a flexible peptide linker comprising the amino acid sequence (GGGGS)₃ (SEQ ID NO: 76); and
(iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ (SEQ ID NO: 77) to form a single-chain variable fragment;
wherein VL-1 or VL-3 comprises a VL amino acid sequence selected from any one of SEQ ID NO: 58 or SEQ ID NO: 60, wherein VH-1 or VH-3 comprises a VH amino acid sequence selected from any one of SEQ ID NO: 57 or SEQ ID NO: 59, wherein VH-2 or VH-4 comprises a VH amino acid sequence selected from any one of SEQ ID NO: 61 or SEQ ID NO: 62, and wherein VL-2 or VL-4 comprises a VL amino acid sequence selected from any one of SEQ ID NO: 63 or SEQ ID NO: 64.

5. The multi-specific antibody of claim 4, wherein the first polypeptide chain or the fourth polypeptide chain comprises the amino acid sequence of SEQ ID NO: 1, and wherein the second polypeptide chain or the third polypeptide chain comprises the amino acid sequence of SEQ ID NO: 3; or
wherein the first polypeptide chain or the fourth polypeptide chain comprises the amino acid sequence of SEQ ID NO: 27, and wherein the second polypeptide chain or the third polypeptide chain comprises the amino acid sequence of SEQ ID NO: 29.

6. (canceled)

7. A method for treating a TSHR-positive cancer in a subject in need thereof comprising administering to the subject an effective amount of the multi-specific antibody of claim 1.

8. The method of claim 7, wherein the TSHR-positive cancer is thyroid cancer, T lineage acute lymphoblastic leukemia, multiple myeloma, lung cancer, colorectal cancer, gastric cancer, liver cancer, pancreatic cancer, urothelial cancer, breast cancer, or ovarian cancer.

9. A method for treating a TSHR-associated pathology in a subject in need thereof comprising administering to the subject an effective amount of the multi-specific antibody of claim 1, wherein the TSHR-associated pathology is Graves' disease, or thyroid-associated ophthalmopathy (TAO).

10. A method for modulating weight gain in a subject in need thereof comprising administering to the subject an effective amount of the multi-specific antibody of claim 2.

11. A method for decreasing bone remodeling to treat osteoporosis in a subject in need thereof comprising administering to the subject an effective amount of the multi-specific antibody of claim 1.

12. The method of claim 7, further comprising administering to the subject an effective amount of a multi-specific antibody that specifically binds to HER2 and T cells, optionally wherein the multi-specific antibody that specifically binds to HER2 and T cells comprises a light chain amino acid sequence of SEQ ID NO: 54 and a heavy chain amino acid sequence of SEQ ID NO: 56.

13. (canceled)

14. The method of claim 7, wherein the TSHR-positive cancer is resistant to a RET inhibitor, a NTRK inhibitor, an ALK inhibitor, a RAF inhibitor, or a MEK kinase inhibitor.

15. An ex vivo armed T cell that is coated or complexed with an effective amount of the multi-specific antibody of claim 1.

16. The ex vivo armed T cell of claim 15, wherein the T cell that is further coated or complexed with an effective amount of a multi-specific antibody that specifically binds to HER2 and T cells, optionally wherein the multi-specific antibody that specifically binds to HER2 and T cells comprises a light chain amino acid sequence of SEQ ID NO: 54 and a heavy chain amino acid sequence of SEQ ID NO: 56.

17. (canceled)

18. A method for treating a TSHR-positive cancer in a subject in need thereof comprising administering to the subject an effective amount of the ex vivo armed T cell of claim 15.

19. The method of claim 18, wherein the ex vivo armed T cell is a αβ-T cell or a γδ-T cell.

20. The method of claim 18, wherein the ex vivo armed T cell is cryopreserved or freshly harvested from a donor.

21. The method of claim 18, wherein the ex vivo armed T cell is obtained from the subject or a third party donor.

22. The method of claim 18, further comprising administering to the subject an effective amount of a second population of ex vivo armed T cells that is coated or complexed with an effective amount of a multi-specific antibody that specifically binds to HER2 and T cells, wherein the second population of ex vivo armed T cells is distinct from the ex vivo armed T cell.

23. The method of claim 22, wherein the multi-specific antibody present on the second population of ex vivo armed T cells comprises a light chain amino acid sequence of SEQ ID NO: 54 and a heavy chain amino acid sequence of SEQ ID NO: 56.

24. The method of claim 18, wherein the TSHR-positive cancer is resistant to a RET inhibitor, a NTRK inhibitor, an ALK inhibitor, a RAF inhibitor, or a MEK kinase inhibitor.


SEQUENCE LISTING (99 sequences)

SEQ ID NO: 1 - protein, 488 aa, synthetic construct
LTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 2 - DNA, 1464 bp, synthetic construct
  source 1..1464: mol_type = other DNA
ctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggacagccaaaggcagcaccttctgtgaccctgtttccaccctctagcgaggagctgcaggccaataaggccaccctggtgtgcctgatcagcgacttctacccaggagcagtgacagtggcatggaaggccgattcctctccagtgaaggccggcgtggagaccacaaccccctctaagcagagcaacaataagtacgccgccagctcctatctgtccctgacccctgagcagtggaagtctcaccggtcttatagctgccaggtgacacacgagggctccacagtggagaagaccgtggccccaacagagtgtactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagtgcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactacagcctggactactggggccagggaacccctgtgacagtgtctagcggagggggaggttcaggtggcggtggatcagggggcggaggaagtggcgggggaggtagtggtggtggtggaagcggaggtggcggctccgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccaga

SEQ ID NO: 3 - protein, 451 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1356 bp, synthetic construct
  source 1..1356: mol_type = other DNA
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaatga

SEQ ID NO: 5 - protein, 518 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSGGGGSGGGGSGGGGSGGGGSLTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 6 - DNA, 1554 bp, synthetic construct
  source 1..1554: mol_type = other DNA
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctggcggtggcggctcaggtggaggcggatctggcggcggcggctctggaggaggcggcagcctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagacatcatcaccatcatcac

SEQ ID NO: 7 - protein, 561 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSGGGGSGGGGSGGGGSGGGGSLTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTPLGDTTHTSGMVSKLSQLQTELLAALLESGLSKEALIQALGEGSGGAP

SEQ ID NO: 8 - DNA, 1701 bp, synthetic construct
  source 1..1701: mol_type = other DNA
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctggcggtggcggctcaggtggaggcggatctggcggcggcggctctggaggaggcggcagcctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagaaccccactgggcgataccacacacacaagcggcatggtgtccaagctgtctcagctgcagaccgagctgctggccgctctgctggagagcggactgtccaaggaggccctgatccaggctctgggagagggcagcggcggcgctccccaccaccaccaccaccac

SEQ ID NO: 9 - protein, 759 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSGGGGSGGGGSGGGGSGGGGSLTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGGGGSGGGGSGGGGSGGGGPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 2210 bp, synthetic construct
  source 1..2210: mol_type = other DNA
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctggcggtggcggctcaggtggaggcggatctggcggcggcggctctggaggaggcggcagcctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 11 - protein, 486 aa, synthetic construct
LTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 12 - DNA, 1464 bp, synthetic construct
  source 1..1464: mol_type = other DNA
ctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggacagccaaaggcagcaccatctgtgacactgtttccacccagctccgaggagctgcaggccaataaggccaccctggtgtgcctgatctccgacttctaccctggagcagtgacagtggcatggaaggccgattctagccctgtgaaggccggcgtggagaccacaaccccaagcaagcagtccaacaataagtacgccgcctcctcttatctgtccctgacccccgagcagtggaagtctcaccggtcctattcttgccaggtgacacacgagggcagcacagtggagaagaccgtggcccctacagagtgtactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagtgcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactacagcctggactactggggccagggaacccctgtgacagtgtctagcggagggggaggttcaggtggcggtggatcagggggcggaggaagtggcgggggaggtagtggtggtggtggaagcggaggtggcggctccgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccaga

SEQ ID NO: 13 - protein, 451 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 14 - DNA, 1356 bp, synthetic construct; identical to SEQ ID NO: 4
  source 1..1356: mol_type = other DNA

SEQ ID NO: 15 - protein, 215 aa, synthetic construct
LTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 16 - DNA, 645 bp, synthetic construct
  source 1..645: mol_type = other DNA
ctgacagtgctgacccagccaccttctgtgagcggcgcccctaggcagagagtgaccatctcctgctctggcaacagctccaatatcggcaacaatgccgtgaactggtaccagcagctgcctggcaaggccccaaagctgctgatctactatgacgatcagctgccttccggcgtgtctgacaggtttagcggctcccgctctggcaccagcgcctccctggccatccggggcctgcagagcgaggacgaggccgattactattgcacatcctgggacgatagcctggattcccagctgttcggaggaggaacaaggctgaccgtgctgggacagccaaaggcagcaccatctgtgacactgtttccacccagctccgaggagctgcaggccaataaggccaccctggtgtgcctgatctccgacttctaccctggagcagtgacagtggcatggaaggccgattctagccctgtgaaggccggcgtggagaccacaaccccaagcaagcagtccaacaataagtacgccgcctcctcttatctgtccctgacccccgagcagtggaagtctcaccggtcctattcttgccaggtgacacacgagggcagcacagtggagaagaccgtggcccctacagagtgt

SEQ ID NO: 17 - protein, 723 aa, synthetic construct
  VARIANT 423: note = Any amino acid
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQXGNVFSCSVMHEALHNHYTQKSLSLSPGTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 18 - DNA, 2169 bp, synthetic construct
  source 1..2169: mol_type = other DNA
  misc_difference 1269: note = a, c, t, g, unknown, or other
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcanggcaacgtgttctcctgctccgtgatgcacgaggccctgcacaaccactacacccagaagtccctgtccctgagccccggcactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagtgcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactacagcctggactactggggccagggaacccctgtgacagtgtctagcggagggggaggttcaggtggcggtggatcagggggcggaggaagtggcgggggaggtagtggtggtggtggaagcggaggtggcggctccgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccaga

SEQ ID NO: 19 - protein, 215 aa, synthetic construct; identical to SEQ ID NO: 15

SEQ ID NO: 20 - DNA, 645 bp, synthetic construct; identical to SEQ ID NO: 16
  source 1..645: mol_type = other DNA

SEQ ID NO: 21 - protein, 451 aa, synthetic construct
QVQLVQSGAEVKKPGESLKISCRGSGYRFTSYWINWVRQLPGKGLEWMGRIDPTDSYTNYSPSFKGHVTVSADKSINTAYLQWSSLKASDTGMYYCARLEPGYSSTWSVNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 22 - DNA, 1356 bp, synthetic construct
  source 1..1356: mol_type = other DNA
caggtgcagctggtgcagagcggagcagaggtgaagaagccaggcgagtctctgaagatcagctgccggggctccggctacagattcacctcctattggatcaactgggtgaggcagctgccaggcaagggcctggagtggatgggaaggatcgaccccaccgattcttacacaaactatagcccttcctttaagggccacgtgaccgtgtctgccgacaagagcatcaatacagcctacctgcagtggagctccctgaaggcctccgataccggcatgtactattgtgcccggctggagcccggctattctagcacatggtctgtgaattggggccagggcaccctggtgacagtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaggctgaccgtggacaagtcccggtggcagcagggcaacgtgttctcctgctccgtgatgcacgaggccctgcacaaccactacacccagaagtccctgtccctgagccccggcaaatga

SEQ ID NO: 23 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 639 bp, synthetic construct
  source 1..639: mol_type = other DNA
gatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccagaaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc

SEQ ID NO: 25 - protein, 449 aa, synthetic construct
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 26 - DNA, 1347 bp, synthetic construct
  source 1..1347: mol_type = other DNA
caggtgcagctggtgcagtctggtggcggcgtggtgcagcctggcagatccctgcggctgtcctgcaaggcctccggctacaccttcacccggtacaccatgcactgggtccgacaggctccaggcaagggcctggaatggatcggctacatcaacccctcccggggctacaccaactacaaccagaagttcaaggaccggttcaccatctcccgggacaactccaagaacaccgcctttctgcagatggactctctgcggcccgaggataccggcgtgtacttctgcgcccggtactacgacgaccactactccctggactactggggccagggcacccctgtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - protein, 488 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 28 - DNA, 1464 bp, synthetic construct
  source 1..1464: mol_type = other DNA
cagagcgtgctgacccagccacctagcgtgtccgccgcaccaggacagaaggtgacaatctcttgcagcggcagctcctctgacatcggctctaactacgtgagctggtatcagcagtttcctggcaccgccccaaagctgctgatctacgacaacaataagcggcccagcgccatccctgatagattctccggctctaagagcggcacatccgccaccctgggaatcacaggactgcagaccggcgacgaggcagattactattgcggcacctgggattccaggctgggaatcgccgtgttcggaggaggaacacagctgaccgtgctgggacagccaaaggcagcaccatctgtgacactgtttccacccagctccgaggagctgcaggccaataaggccaccctggtgtgcctgatctccgacttctaccctggagcagtgacagtggcatggaaggccgattctagccctgtgaaggccggcgtggagaccacaaccccaagcaagcagtccaacaataagtacgccgcctcctcttatctgtccctgacccccgagcagtggaagtctcaccggtcctattcttgccaggtgacacacgagggcagcacagtggagaagaccgtggcccctacagagtgtactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagtgcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactacagcctggactactggggccagggaacccctgtgacagtgtctagcggagggggaggttcaggtggcggtggatcagggggcggaggaagtggcgggggaggtagtggtggtggtggaagcggaggtggcggctccgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccaga

SEQ ID NO: 29 - protein, 449 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1350 bp, synthetic construct
  source 1..1350: mol_type = other DNA
gaggtgcagctggtgcagtctggcgccgaggtgaagaagcctggccagtctctgaagatcagctgcaaggccagcggctactccctgaccgacaactggatcggatgggtgcggcagaagccaggcaagggcctggagtggatgggcatcatctacccaggcgacagcgatacaagatattccccctctttccagggccaggtgaccatctccgccgataagtctatcaatacagcctatctgcagtggagctccctgaaggcctccgacaccgccatctactattgcgtgggcctggattggaactacaatccactgaggtattggggacccggcaccctggtgacagtgtctagcgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaatga

SEQ ID NO: 31 - protein, 521 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSGGGGSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKRGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 32 - DNA, 1563 bp, synthetic construct
  source 1..1563: mol_type = other DNA
gaagtgcagctggtgcagtccggcgccgaggtgaagaagcctgggcaatctctgaagatcagctgcaaggcctcaggatatagcctgaccgacaattggatcggctgggtgcggcagaagcccggcaagggcttggagtggatgggcatcatctaccctggcgattccgacacccggtacagccctagcttccagggccaagtgaccatctccgccgataagtccatcaacaccgcctacctgcagtggtccagcctgaaagcctctgatacagccatctactactgcgtgggcctggactggaactacaaccctctgagatactggggacctggaaccctcgtcaccgtgtcctctggcggtggcggctcaggtggaggcggatctggcggcggcggctctggaggaggcggcagccagtctgtgctgacccagcctccttccgtgtctgctgctccaggccagaaagtgacaatctcctgcagcggctccagctccgatatcggcagcaactacgtgtcctggtaccagcagttccccggcacagctcctaagctgctgatctacgacaacaacaagagaccttctgctattcccgacagatttagcggaagcaagagcggcacatctgccacactgggcatcaccggcctgcagaccggcgacgaggccgactactactgtggcacctgggacagcagactgggcatcgccgtcttcggcggcggcacccagctgaccgtgctgggccagcccaagcggggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagacatcatcaccatcatcac

SEQ ID NO: 33 - protein, 564 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSGGGGSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKRGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTPLGDTTHTSGMVSKLSQLQTELLAALLESGLSKEALIQALGEGSGGAP

SEQ ID NO: 34 - DNA, 1710 bp, synthetic construct
  source 1..1710: mol_type = other DNA
gaagtgcagctggtgcagtccggcgccgaggtgaagaagcctgggcaatctctgaagatcagctgcaaggcctcaggatatagcctgaccgacaattggatcggctgggtgcggcagaagcccggcaagggcttggagtggatgggcatcatctaccctggcgattccgacacccggtacagccctagcttccagggccaagtgaccatctccgccgataagtccatcaacaccgcctacctgcagtggtccagcctgaaagcctctgatacagccatctactactgcgtgggcctggactggaactacaaccctctgagatactggggacctggaaccctcgtcaccgtgtcctctggcggtggcggctcaggtggaggcggatctggcggcggcggctctggaggaggcggcagccagtctgtgctgacccagcctccttccgtgtctgctgctccaggccagaaagtgacaatctcctgcagcggctccagctccgatatcggcagcaactacgtgtcctggtaccagcagttccccggcacagctcctaagctgctgatctacgacaacaacaagagaccttctgctattcccgacagatttagcggaagcaagagcggcacatctgccacactgggcatcaccggcctgcagaccggcgacgaggccgactactactgtggcacctgggacagcagactgggcatcgccgtcttcggcggcggcacccagctgaccgtgctgggccagcccaagcggggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagaaccccactgggcgataccacacacacaagcggcatggtgtccaagctgtctcagctgcagaccgagctgctggccgctctgctggagagcggactgtccaaggaggccctgatccaggctctgggagagggcagcggcggcgctccccaccaccaccaccaccac

SEQ ID NO: 35 - protein, 762 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSGGGGSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKRGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGGGGSGGGGSGGGGSGGGGPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 2219 bp, synthetic construct
  source 1..2219: mol_type = other DNA
gaagtgcagctggtgcagtccggcgccgaggtgaagaagcctgggcaatctctgaagatcagctgcaaggcctcaggatatagcctgaccgacaattggatcggctgggtgcggcagaagcccggcaagggcttggagtggatgggcatcatctaccctggcgattccgacacccggtacagccctagcttccagggccaagtgaccatctccgccgataagtccatcaacaccgcctacctgcagtggtccagcctgaaagcctctgatacagccatctactactgcgtgggcctggactggaactacaaccctctgagatactggggacctggaaccctcgtcaccgtgtcctctggcggtggcggctcaggtggaggcggatctggcggcggcggctctggaggaggcggcagccagtctgtgctgacccagcctccttccgtgtctgctgctccaggccagaaagtgacaatctcctgcagcggctccagctccgatatcggcagcaactacgtgtcctggtaccagcagttccccggcacagctcctaagctgctgatctacgacaacaacaagagaccttctgctattcccgacagatttagcggaagcaagagcggcacatctgccacactgggcatcaccggcctgcagaccggcgacgaggccgactactactgtggcacctgggacagcagactgggcatcgccgtcttcggcggcggcacccagctgaccgtgctgggccagcccaagcggggcggaggaggctccggaggcggcggctctggcggagggggatcccaggtgcagctggtccagtctggaggaggagtggtccagccagggagatcactgcgcctgtcctgtaaggccagcggctacactttcaccagatatactatgcattgggtgcgccaggctccagggaaaggtctggagtggatcggttacattaacccctccaggggatacaccaactataatcagaagttcaaagacaggttcaccatctctcgtgataacagtaagaataccgcattcctgcagatggactctctgcgacccgaagatactggcgtgtatttttgcgcccgttactatgacgatcattactccctggactattggggccagggaacacctgtgactgtcagctctggtggaggagggagcggtggaggagggtctggtggaggagggagtggtggaggagggtcagacatccagatgacacagagcccaagttcactgagcgcatctgtgggcgatcgggtcacaattacttgctctgcctccagctctgtgagttacatgaactggtatcagcagacacctggaaaggctccaaaaagatggatctacgatacttcaaaactggcatccggagtgccaagccgcttcagtgggtcaggttccggcaccgactatacctttacaatcagttcactgcagcctgaggatattgctacatactattgtcagcagtggtccagcaatccattcaccttcggacaggggacaaaactgcagattacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 37 - protein, 486 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 38 - DNA, 1464 bp, synthetic construct; identical to SEQ ID NO: 28
  source 1..1464: mol_type = other DNA

SEQ ID NO: 39 - protein, 449 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 40 - DNA, 1350 bp, synthetic construct; identical to SEQ ID NO: 30
  source 1..1350: mol_type = other DNA

SEQ ID NO: 41 - protein, 215 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 42 - DNA, 645 bp, synthetic construct
  source 1..645: mol_type = other DNA
cagagcgtgctgacccagccacctagcgtgtccgccgcaccaggacagaaggtgacaatctcttgcagcggcagctcctctgacatcggctctaactacgtgagctggtatcagcagtttcctggcaccgccccaaagctgctgatctacgacaacaataagcggcccagcgccatccctgatagattctccggctctaagagcggcacatccgccaccctgggaatcacaggactgcagaccggcgacgaggcagattactattgcggcacctgggattccaggctgggaatcgccgtgttcggaggaggaacacagctgaccgtgctgggacagccaaaggcagcaccatctgtgacactgtttccacccagctccgaggagctgcaggccaataaggccaccctggtgtgcctgatctccgacttctaccctggagcagtgacagtggcatggaaggccgattctagccctgtgaaggccggcgtggagaccacaaccccaagcaagcagtccaacaataagtacgccgcctcctcttatctgtccctgacccccgagcagtggaagtctcaccggtcctattcttgccaggtgacacacgagggcagcacagtggagaagaccgtggcccctacagagtgt

SEQ ID NO: 43 - protein, 721 aa, synthetic construct
  VARIANT 421: note = Any amino acid
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQXGNVFSCSVMHEALHNHYTQKSLSLSPGTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 44 - DNA, 2163 bp, synthetic construct
  source 1..2163: mol_type = other DNA
  misc_difference 1263: note = a, c, t, g, unknown, or other
gaggtgcagctggtgcagtctggcgccgaggtgaagaagcctggccagtctctgaagatcagctgcaaggccagcggctactccctgaccgacaactggatcggatgggtgcggcagaagccaggcaagggcctggagtggatgggcatcatctacccaggcgacagcgatacaagatattccccctctttccagggccaggtgaccatctccgccgataagtctatcaatacagcctatctgcagtggagctccctgaaggcctccgacaccgccatctactattgcgtgggcctggattggaactacaatccactgaggtattggggacccggcaccctggtgacagtgtctagcgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcanggcaacgtgttctcctgctccgtgatgcacgaggccctgcacaaccactacacccagaagtccctgtccctgagccccggcactagtggcggcggaggatctggcggaggtggaagtgggggaggcggatctcaggtgcagctggtgcagagtggtggcggagtggtgcagcctggcagatccctgagactgtcttgcaaggccagcggctacaccttcacccggtacaccatgcactgggtgcgacaggcccctggcaagtgcctggaatggatcggctacatcaacccctcccggggctacaccaactacaaccagaagttcaaggaccggttcaccatcagccgggacaactccaagaacaccgcctttctgcagatggactccctgcggcctgaggataccggcgtgtacttttgcgcccggtactacgacgaccactacagcctggactactggggccagggaacccctgtgacagtgtctagcggagggggaggttcaggtggcggtggatcagggggcggaggaagtggcgggggaggtagtggtggtggtggaagcggaggtggcggctccgatatccagatgacccagtccccctccagcctgtctgcctctgtgggagacagagtgacaattacatgctccgccagctccagcgtgtcctacatgaattggtatcagcagacccctggcaaggctcccaagcggtggatctacgacacctccaagctggcctccggcgtgccctccagattttctggcagcggctccggcacagactatacctttacaatcagctccctgcagcccgaagatatcgccacctactactgccagcagtggtcctccaaccccttcaccttcggctgcggcacaaagctgcagatcaccaga

SEQ ID NO: 45 - protein, 215 aa, synthetic construct; identical to SEQ ID NO: 41

SEQ ID NO: 46 - DNA, 645 bp, synthetic construct; identical to SEQ ID NO: 42
  source 1..645: mol_type = other DNA

SEQ ID NO: 47 - protein, 449 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 48 - DNA, 1350 bp, synthetic construct
  source 1..1350: mol_type = other DNA
gaggtgcagctggtgcagtctggcgccgaggtgaagaagcctggccagtctctgaagatcagctgcaaggccagcggctactccctgaccgacaactggatcggatgggtgcggcagaagccaggcaagggcctggagtggatgggcatcatctacccaggcgacagcgatacaagatattccccctctttccagggccaggtgaccatctccgccgataagtctatcaatacagcctatctgcagtggagctccctgaaggcctccgacaccgccatctactattgcgtgggcctggattggaactacaatccactgaggtattggggacccggcaccctggtgacagtgtctagcgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaggctgaccgtggacaagtcccggtggcagcagggcaacgtgttctcctgctccgtgatgcacgaggccctgcacaaccactacacccagaagtccctgtccctgagccccggcaaatga

SEQ ID NO: 49 - protein, 213 aa, synthetic construct; identical to SEQ ID NO: 23

SEQ ID NO: 50 - DNA, 639 bp, synthetic construct; identical to SEQ ID NO: 24
  source 1..639: mol_type = other DNA

SEQ ID NO: 51 - protein, 449 aa, synthetic construct; identical to SEQ ID NO: 25

SEQ ID NO: 52 - DNA, 1347 bp, synthetic construct; identical to SEQ ID NO: 26
  source 1..1347: mol_type = other DNA

SEQ ID NO: 53 - DNA, 1461 bp, synthetic construct
  source 1..1461: mol_type = other DNA
gacatccagatgacccagagcccttccagcctgtccgcctctgtgggcgacagagtgaccatcacctgtcgggcctcccaggacgtgaacaccgccgtggcttggtatcagcagaagcccggcaaggcccccaagctgctgatctactccgcctccttcctgtactccggcgtgccctccagattctccggcagcagatctggcaccgactttaccctgaccatctccagcctgcagcccgaggacttcgccacctactactgccagcagcactacaccaccccccccacctttggccagggcaccaaggtggaaatcaagcggaccgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgcactagtggcggcggaggatctggcggaggtggaagcggagggggaggatctcaggtgcagctggtgcagagcggaggcggagtggtgcagcctggcagatccctgagactgtcctgcaaggcctccggctacaccttcacccggtacaccatgcactgggtgcgacaggcccctggcaagggcctggaatggatcggctacatcaacccctcccggggctacaccaactacaaccagaagttcaaggaccggttcaccatctcccgggacaactccaagaacaccgcctttctgcagatggactccctgcggcctgaggataccggcgtgtacttctgcgcccggtactacgacgaccactactccctggactactggggccagggaacccctgtgacagtgtcatctggtggcggaggaagtgggggaggcggatcaggtggtggtggatcaggcgggggaggttcagggggtggcggttctgggggagggggctctgatattcagatgactcagagcccttccagcctgagcgcctccgtgggagatcgcgtgacaattacctgctctgcctcctcctccgtgtcttacatgaattggtatcagcagacccctgggaaggctcctaagcggtggatctacgacacctccaagctggcctctggcgtgcccagcaggttttctggctccggcagcggcacagattataccttcaccatcagctccctgcagccagaagatatcgctacctattattgtcagcagtggtcctccaaccctttcaccttcggccagggcacaaagctgcagatcacaaga

SEQ ID NO: 54 - protein, 487 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 55 - DNA, 1350 bp, synthetic construct
  source 1..1350: mol_type = other DNA
gaggtgcagctggtggaatctggcggaggactggtgcagcctggcggctctctgagactgtcttgtgccgcctccggcttcaacatcaaggacacctacatccactgggtgcgacaggcccctggcaagggactggaatgggtggccagaatctaccccaccaacggctacaccagatacgccgactctgtgaagggccggttcaccatctccgccgacacctccaagaacaccgcctacctgcagatgaactccctgcgggccgaggacaccgccgtgtactactgtagtagatggggaggcgacggcttctacgccatggactattggggccagggcaccctcgtgaccgtgtcctctgcttctaccaagggcccctctgtgtttcctctggccccctccagcaagtccacctctggtggaacagccgccctgggctgcctcgtgaaggactactttcccgagcccgtgaccgtgtcctggaactctggcgctctgacctctggcgtgcacaccttccctgctgtgctgcagtctagcggcctgtactccctgtcctccgtcgtgacagtgccctccagctctctgggcacccagacctacatctgcaacgtgaaccacaagccctccaataccaaggtggacaagcgggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacgcctccacctaccgggtggtgtccgtgctgacagtgctgcaccaggactggctgaacggcaaagagtacaagtgcgccgtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgaccgtggacaagtcccggtggcagcagggcaacgtgttctcctgctccgtgatgcacgaggccctgcacaaccactacacccagaagtccctgtccctgagccccggcaaa

SEQ ID NO: 56 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 121 aa, synthetic construct [sequence omitted]

SEQ ID NO: 58 - protein, 110 aa, synthetic construct
LTVLTQPPSVSGAPRQRVTISCSGNSSNIGNNAVNWYQQLPGKAPKLLIYYDDQLPSGVSDRFSGSRSGTSASLAIRGLQSEDEADYYCTSWDDSLDSQLFGGGTRLTVL

SEQ ID NO: 59 - protein, 119 aa, synthetic construct [sequence omitted]

SEQ ID NO: 60 - protein, 110 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVL

SEQ ID NO: 61 - protein, 119 aa, synthetic construct
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 62 - protein, 119 aa, synthetic construct
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 63 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 64 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 65 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 66 - protein, 330 aa, Homo sapiens [sequence omitted]

SEQ ID NO: 67 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 68 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 69 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 70 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 72 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 73 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 764 aa, Homo sapiens
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHDMRQGLHNMEDVYELIENSHLTPKKQGQISEEYMQTVL

SEQ ID NO: 75 - protein, 239 aa, Homo sapiens [sequence omitted]

SEQ ID NO: 76 - protein, 15 aa, synthetic construct
GGGGSGGGGSGGGGS

SEQ ID NO: 77 - protein, 30 aa, synthetic construct
GGGGSGGGGSGGGGSGGGGSGGGGSGGGGS

SEQ ID NO: 78 - protein, 75 aa, synthetic construct
  VARIANT 1..75: note = This sequence may encompass 1-15 GGGGS repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 79 - protein, 5 aa, synthetic construct
GGGGS

SEQ ID NO: 80 - protein, 6 aa, synthetic construct
HHHHHH

SEQ ID NO: 81 - protein, 4 aa, synthetic construct
  SITE 1: note = Dodecane tetraacetic acid Phenylalanine
  SITE 2: note = Lysine histaminyl-succinyl-glycine
  SITE 3: note = D-Tyrosine
  SITE 4: note = Lysine histaminyl-succinyl-glycine amine
FKYK

SEQ ID NO: 82 - protein, 4 aa, synthetic construct
  SITE 1: note = Acetyl Lysine histaminyl-succinyl-glycine
  SITE 2: note = D-Tyrosine
  SITE 3: note = Lysine histaminyl-succinyl-glycine
  SITE 4: note = Lysine 3-thiosemicarbazonyl-glyoxylcysteinyl amine
KYKK

SEQ ID NO: 83 - protein, 4 aa, synthetic construct
  SITE 1: note = Dodecane tetraacetic acid D-Aspartic Acid
  SITE 2: note = D-Lysine histaminyl-succinyl-glycine
  SITE 3: note = D-Aspartic Acid
  SITE 4: note = D-Lysine histaminyl-succinyl-glycine amine
DKDK

SEQ ID NO: 84 - protein, 4 aa, synthetic construct
  SITE 1: note = Dodecane tetraacetic acid D-Glutamic Acid
  SITE 2: note = D-Lysine histaminyl-succinyl-glycine
  SITE 3: note = D-Glutamic Acid
  SITE 4: note = D-Lysine histaminyl-succinyl-glycine amine
EKEK

SEQ ID NO: 85 - protein, 4 aa, synthetic construct
  SITE 1: note = Dodecane tetraacetic acid D-Tyrosine
  SITE 2: note = D-Lysine histaminyl-succinyl-glycine
  SITE 3: note = D-Glutamic Acid
  SITE 4: note = D-Lysine histaminyl-succinyl-glycine amine
YKEK

SEQ ID NO: 86 - protein, 4 aa, synthetic construct
  SITE 1: note = Dodecane tetraacetic acid D-Alanine
  SITE 2: note = D-Lysine histaminyl-succinyl-glycine
  SITE 3: note = D-Glutamic Acid
  SITE 4: note = D-Lysine histaminyl-succinyl-glycine amine
AKEK

SEQ ID NO: 87 - protein, 4 aa, synthetic construct
  SITE 1: note = Dodecane tetraacetic acid D-Phenylalanine
  SITE 2: note = D-Lysine histaminyl-succinyl-glycine
  SITE 3: note = D-Tyrosine
  SITE 4: note = D-Lysine histaminyl-succinyl-glycine amine
FKYK

SEQ ID NO: 88 - protein, 4 aa, synthetic construct
  SITE 1: note = Acetyl D-Phenylalanine
  SITE 2: note = D-Lysine dodecane tetraacetic acid
  SITE 3: note = D-Tyrosine
  SITE 4: note = D-Lysine dodecane tetraacetic acid amine
FKYK

SEQ ID NO: 89 - protein, 4 aa, synthetic construct
  SITE 1: note = Acetyl D-Phenylalanine
  SITE 2: note = D-Lysine diethylenetriaminepentaacetic acid
  SITE 3: note = D-Tyrosine
  SITE 4: note = D-Lysine diethylenetriaminepentaacetic acid amine
FKYK

SEQ ID NO: 90 - protein, 4 aa, synthetic construct
  SITE 1: note = Acetyl D-Phenylalanine
  SITE 2: note = D-Lysine benzyl-diethylenetriaminepentaacetic acid
  SITE 3: note = D-Tyrosine
  SITE 4: note = D-Lysine benzyl-diethylenetriaminepentaacetic acid amine
FKYK

SEQ ID NO: 91 - protein, 4 aa, synthetic construct
  SITE 1: note = Acetyl D-Lysine histaminyl-succinyl-glycine
  SITE 2: note = D-Tyrosine
  SITE 3: note = D-Lysine histaminyl-succinyl-glycine
  SITE 4: note = D-Lysine 3-thiosemicarbazonyl-glyoxylcysteinyl amine
KYKK

SEQ ID NO: 92 - protein, 5 aa, synthetic construct
  SITE 1: note = Dodecane tetraacetic acid D-Phenylalanine
  SITE 2: note = D-Lysine histaminyl-succinyl-glycine
  SITE 3: note = D-Tyrosine
  SITE 4: note = D-Lysine histaminyl-succinyl-glycine
  SITE 5: note = D-Lysine 3-thiosemicarbazonyl-glyoxylcysteinyl amine
FKYKK

SEQ ID NO: 93 - protein, 5 aa, synthetic construct
  SITE 1: note = 3-thiosemicarbazonyl-glyoxylcysteinyl D-Phenylalanine
  SITE 2: note = D-Lysine histaminyl-succinyl-glycine
  SITE 3: note = D-Tyrosine
  SITE 4: note = D-Lysine histaminyl-succinyl-glycine
  SITE 5: note = D-Lysine dodecane tetraacetic acid amine
FKYKK

SEQ ID NO: 94 - protein, 5 aa, synthetic construct
  SITE 1: note = D-3-thiosemicarbazonyl-glyoxylcysteinyl cysteine
  SITE 2: note = D-Glutamic Acid
  SITE 3: note = D-Lysine histaminyl-succinyl-glycine
  SITE 4: note = D-Glutamic Acid
  SITE 5: note = D-Lysine histaminyl-succinyl-glycine amine
CEKEK

SEQ ID NO: 95 - protein, 4 aa, synthetic construct
  SITE 1: note = 3-thiosemicarbazonyl-glyoxylcysteinyl D-Glutamic Acid
  SITE 2: note = D-Lysine histaminyl-succinyl-glycine
  SITE 3: note = D-Glutamic Acid
  SITE 4: note = D-Lysine histaminyl-succinyl-glycine amine
EKEK

SEQ ID NO: 96 - protein, 6 aa, synthetic construct
  SITE 1: note = Acetyl D-Cysteine
  SITE 2: note = D-Lysine dodecane tetraacetic acid
  SITE 3: note = D-Tyrosine
  SITE 4: note = D-Alanine
  SITE 5: note = D-Lysine dodecane tetraacetic acid
  SITE 6: note = D-Cysteine amine
CKYAKC

SEQ ID NO: 97 - protein, 4 aa, synthetic construct
  SITE 1: note = Acetyl D-Cysteine
  SITE 2: note = D-Lysine diethylenetriaminepentaacetic acid
  SITE 3: note = D-Tyrosine
  SITE 4: note = D-Lysine diethylenetriaminepentaacetic acid amine
CKYK

SEQ ID NO: 98 - protein, 4 aa, synthetic construct
  SITE 1: note = Acetyl D-Lysine diethylenetriaminepentaacetic acid
  SITE 2: note = D-Tyrosine
  SITE 3: note = D-Lysine diethylenetriaminepentaacetic acid
  SITE 4: note = D-Lysine 3-thiosemicarbazonyl-glyoxylcysteinyl amine
KYKK

SEQ ID NO: 99 - protein, 4 aa, synthetic construct
  SITE 1: note = Acetyl D-Lysine dodecane tetraacetic acid
  SITE 2: note = D-Tyrosine
  SITE 3: note = D-Lysine dodecane tetraacetic acid
  SITE 4: note = D-Lysine 3-thiosemicarbazonyl-glyoxylcysteinyl amine
KYKK